(19) United States
(12) Patent Application Publication
(10) Pub. No.: US 2025/0170127 A1
(43) Pub. Date: May 29, 2025

(54) RE-SENSITIZING MULTIDRUG-RESISTANT (MDR) GRAM-NEGATIVE BACTERIA TO COLISTIN USING IONOPHORES

(71) Applicant: The Regents of the University of Colorado, a body corporate, Denver, CO (US)

(72) Inventors: Xiang Wang, Boulder, CO (US); Yuefeng Gao, Superior, CO (US); Somnath Dutta, Boulder, CO (US); Nianzi Liu, Boulder, CO (US)

(21) Appl. No.: 18/840,354

(22) PCT Filed: Feb. 21, 2023
(86) PCT No.: PCT/US2023/062944

§ 371 Date: Aug. 21, 2024

Related U.S. Application Data
(60) Provisional application No. 63/312,186, filed on Feb. 21, 2022.

(51) Int. Cl. A61K 31/4985; A61K 38/12; A61P 31/04; C07D 498/04

(52) U.S. Cl. CPC A61K 31/4985; A61K 38/12; A61P 31/04; C07D 498/04

(57) ABSTRACT

The present invention is directed to novel resistance modifying agents (RMAs) compounds configured to potentiate antibiotic compounds, such as polymyxin antibiotics, including colistin, directed to multi-drug resistance (MDR) gram-negative bacteria.

### CROSS-REFERENCE TO RELATED APPLICATIONS

This International PCT application claims the benefit of and priority to U.S. Provisional Patent Application No. 63/312,186 filed Feb. 21, 2022, which is incorporated herein by reference in its entirety.

### STATEMENT OF GOVERNMENT INTEREST

This invention was made with government support under grant number AI121581 awarded by the National Institutes of Health. The government has certain rights in the invention.

### TECHNICAL FIELD

The present inventions relates to small-molecule compounds and their use as both novel resistance modifying agents (RMAs). In particular, the present invention relates to novel compounds that are capable of re-sensitizing multidrug resistant gram-negative bacteria to polymyxin class of antibiotics, and in particular colistin. Additional embodiments include synthesis of these novel RMA compounds, and their therapeutic applications.

### BACKGROUND

Antibiotic resistance is a growing world health concern. In 2019, the U.S. Centers For Disease Control (CDC) estimated over 2.8 million antibiotic-resistant infections occur and more than 35,000 people die in the United States each year as a result. The predicted annual global death toll by 2050 is 10 million. Antibiotic resistant phenotypes have risen rapidly among clinically relevant pathogens. Gram-negative bacteria are of particular concern, including multidrug-resistant (MDR) Gram-negative bacteria Pseudomonas aeruginosa, Acinetobacter baumannii, and Enterobacteriaceae spp. Treatment options for resistant Gram-negative bacteria are very limited, leading to poor patient prognosis. These pathogens are often associated with severe nosocomial infections with 30-40% of mortality rates. Colistin is currently used as an essential last-resort treatment in clinic for infections with MDR Gram-negative bacteria. In 2019, World Health Organization (WHO) designated colistin as “highest priority” among the “critically important antimicrobials for human medicine”.

Colistin (a.k.a., polymyxin E) is a cationic antimicrobial peptide of the polymyxin family. Its antibacterial activity depends on electrostatic interactions between its positively charged moiety and negatively charged phosphate groups of Lipid A, a core component of lipopolysaccharide in the outer membrane of Gram-negative bacteria. Colistin displaces divalent cations from the outer membrane and in turn disrupts its integrity. However, most Gram-negative bacteria, including E. coli, K. pneumonia, P. aeruginosa, A. baumannii and Salmonella spp., have evolved or acquired colistin resistance mechanisms that involve cationic additions of 4-amino-arabinose and/or phosphoethanolamine (pEtN) to the phosphate groups in Lipid A. Both modifications impair the binding of colistin and reduce its activity. pEtN modification is responsible for resistance in A. baumannii and has rapidly spread across many Gram-negative bacteria, such as E. coli, K. pneumonia, and Salmonella spp., via the Mobilized Colistin Resistance (mcr) plasmids.

The discovery of novel antimicrobial agents for Gram-negative bacteria has been proven particularly difficult, mainly due to the prevalence of MDR efflux pumps and the extra barrier of their outer membrane. A variety of novel approaches have been investigated to overcome colistin resistance, including the development of novel polymyxin analogs and the use of combinations of colistin with other clinically approved antibiotics (e.g., carbapenems, rifampicin, tigecycline, and fosfomycin). Several novel small molecules have also been discovered to reverse colistin resistance in mcr-positive bacteria and showed promising results in vitro. Encouraged by previous success on the development of a variety of resistance-modifying agents (RMAs) that re-sensitize methicillin-resistant Staphylococcus aureus (MRSA) to beta-lactam antibiotics, the present inventors initiated a screening approach to re-sensitize MDR Gram-negative bacteria to colistin. As described below, the present invention includes the discovery of a highly active and selective RMA for colistin-resistant Gram-negative bacteria.

### SUMMARY OF THE INVENTION

One aspects of the invention provides one or more small molecule RMAs derived from ionophore compounds. Ionophores are small-molecules that transport ions such as proton, Na⁺, K⁺, Ca²⁺, and Mg²⁺, across the lipid bilayer of membranes. As noted above, RMAs may target non-essential, resistance-conferring genes and restore antibiotic sensitivity of a bacteria. A notable advantage of RMAs is that they are capable of extending the market lifespan of known antibiotics that have already been optimized for large-scale production with well-studied toxicity profiles. In one particular aspect, the present inventors, using a bacterial whole-cell screen of a fragment-based library, identified novel small molecule compounds that re-sensitize MDR E. coli AR-0493 to colistin with low mammalian toxicity. Interestingly, post-screening validation studies identified a highly related, yet distinct compound as the actual substance responsible for the activity. Further studies showed that this novel resistance-modifying agent is not only very potent, but also highly selective to potentiate the activity of polymyxin family antibiotics in a wide range of MDR Gram-negative bacteria. Thus, the RMAs of the invention may be further developed as a combination therapy to prolong the life span of colistin.

In a preferred aspect, a variety of novel RMA compounds may be synthesized and used to potentiate antibiotic compounds, such as representative polymyxin antibiotics such as colistin directed to MDR gram-negative bacteria.

Another aspect of the present invention includes a novel class of RMAs, that may, in one preferred embodiment, selectively re-sensitizes antibiotics, such as polymyxin family antibiotics, and in particular colistin against in a wide range of MDR Gram-negative bacteria. The compounds of the invention can be used in combination with one or more antibiotics, such as the polymyxin family antibiotics, and in particular colistin, to treat antibiotic resistant bacterial infections such as those caused by mcr-positive Enterobacteriaceae and chromosomally encoded colistin-resistant A. baumannii.

Another aspect of the present invention includes a novel class of RMAs, selected from the group consisting of one or more compounds of the invention as described below, in one preferred embodiment, that selectively re-sensitizes antibiotics, such as polymyxin family antibiotics, and in particular colistin against in a wide range of MDR Gram-negative bacteria. The compounds of the invention can be used in combination with one or more antibiotics, such as the polymyxin family antibiotics, and in particular colistin, to treat antibiotic resistant bacterial infections such as those caused by mcr-positive Enterobacteriaceae and chromosomally encoded colistin-resistant A. baumannii.

Another aspect of the present invention includes pharmaceutical compositions containing at least one compound of the invention, that may, in one preferred embodiment, selectively re-sensitize antibiotics, such as polymyxin family antibiotics, and in particular colistin against in a wide range of MDR Gram-negative bacteria. The compounds of the invention can be used in combination with one or more antibiotics, such as the polymyxin family antibiotics, and in particular colistin, to treat antibiotic resistant bacterial infections such as those caused by mcr-positive Enterobacteriaceae and chromosomally encoded colistin-resistant A. baumannii. Another aspect of the present invention includes methods for treating a bacterial infection comprising administering to a subject in need thereof a therapeutically effective amount of one or more compounds of the invention, and an antibiotic, wherein said compound potentiates the activity of said antibiotic. In a preferred aspect, the present invention includes methods for treating a bacterial infection, and preferably a bacterial infection caused by a MDR gram-negative bacteria, comprising administering to a subject in need thereof a therapeutically effective amount of a compound of the invention selected from the group consisting of: RMAs, selected from the group consisting of: one or more compounds of the invention and an antibiotic from the polymyxin family, wherein said compound potentiates the activity of said antibiotic.

In another preferred aspect, the present invention includes methods for treating a bacterial infection, and preferably a bacterial infection caused by a MDR gram-negative bacteria, comprising administering to a subject in need thereof a therapeutically effective amount of a compound of the invention selected from the group consisting of: RMAs, selected from the group consisting of: one or more compounds of the invention and colistin, wherein said compound potentiates the activity of said colistin. In a preferred aspect, the present invention includes methods for treating a bacterial infection caused by mcr-positive Enterobacteriaceae and chromosomally encoded colistin-resistant A. baumannii comprising administering to a subject in need thereof a therapeutically effective amount of a compound of the invention selected from the group consisting of: RMAs, selected from the group consisting of: one or more compounds of the invention and an antibiotic from the polymyxin family, wherein said compound potentiates the activity of said antibiotic.

In another preferred aspect, the present invention includes methods for treating a bacterial infection caused by mcr-positive Enterobacteriaceae and chromosomally encoded colistin-resistant A. baumannii comprising administering to a subject in need thereof a therapeutically effective amount of a compound of the invention selected from the group consisting of: RMAs, selected from the group consisting of: one or more compounds of the invention and colistin, wherein said compound potentiates the activity of said colistin.

Another aspect of the present invention includes methods of synthesizing a compound of the invention. In one preferred aspect, the present invention includes methods of synthesizing a compound of the invention according to Schemes 1-5.

While some of the specific substituents for the various compounds of the invention are disclosed herein, it should be noted that combinations of various groups described herein form other embodiments. In this manner, a variety of compounds are embodied within the present invention.

Additional aspects of the invention may be evidenced from the specification, claims and figures provided below.

### BRIEF DESCRIPTION OF THE FIGURES

FIG. 1. Structures of the top 3 hits from the colistin RMA screen.

FIG. 2. Checkerboard broth microdilution assay showed dose-dependent potentiation of colistin by compound 3 against MDR E. coli. AR-0493. Data represent the mean OD (600 nm) in two biological replicates.

### DETAILED DESCRIPTION OF THE INVENTION

As described herein, the present inventors carried out a colistin RMA screen of a fragment-based library in MDR E. coli using a bacterial whole-cell assay. This screen identified 3 novel potentiators of colistin. Follow-up validation studies discovered that an impurity was actually the best potentiators of colistin. That unexpected “impurity,” identified herein as the Compound of Formula II, also referred to as compound 3, was discovered to be the active substance responsible for the RMA activity. Further evaluation of compound 3 in combination with a variety of antibiotics showed that it selectively potentiates the activity of polymyxin family antibiotics, but not other classes of antibiotics. Evaluation of compound 3 in a wide range of MDR Gram-negative bacteria suggested that it potentiates colistin in both mcr-positive Enterobacteriaceae and chromosomally encoded colistin-resistant A. baumannii strains. In addition, compound 3 has low antibacterial activity and mammalian toxicity on its own. Therefore, compound 3 may be used in combination with colistin or other polymyxins, such as polymyxin B, clinically approved or under development, to treat MDR Gram-negative bacterial infections and prolong the life span of this last-resort antibiotics.

The present inventors further undertook to generate a plurality of derivative based on compound 3, which are generally referred to as compounds or compositions of the invention. In one embodiment, the present inventors generated derivative compounds identified herein. It should be appreciated that combinations of various groups described herein form other preferred embodiments. In this manner, a variety of compounds of the invention are embodied within the present invention. In another embodiment, the present inventors generated one or more compounds of the invention according to schemes 1-5 described herein

In one preferred aspect, a novel RMA compound of the invention is identified according to the compound of Formula I, or a stereoisomer, pharmaceutically acceptable salt thereof.

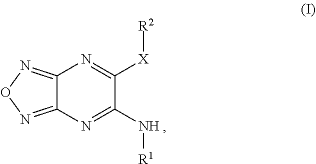

wherein,
X is —H, —OH, —S—, —NHCO—, —NH—, —NH₂, —N(OH)—, or —NHOH;
R₂ is aryl, aryl halide, alkyl, cycloalkane, heteroalkyl, heterocycloalkane, cycloalkane, fused aromatic or fused heteroaromatic, alkyl halide, aromatic, heteroaromatic, amino, amine, all of the foregoing being unsubstituted or unsubstituted, —C(═O)_{R3}, —C(═)OOR₃, —S(═O)R₃, —S(═O)₂R₃, —S(═O)₂NHR₃, Ph, (CN)Ph-, (NO₂)Ph-, (CF₃)Ph-, or absent; and
R₁ aryl, aryl halide, alkyl, cycloalkane, heteroalkyl, heterocycloalkane, cycloalkane, fused aromatic or fused heteroaromatic, alkyl halide, aromatic, heteroaromatic, amino, amine, all of the foregoing being unsubstituted or unsubstituted, —C(═O)R₃, —C(═)OOR₃, —S(═O)R₃, —S(═O)₂R₃, —S(═O)₂NHR₃, Ph, —CN, —NO₂, —CF₃, or absent;
R₃ is H, alkyl, alkoxy, halogen, aromatic, heteroaromatic, cycloalkane, heteroalkyl, heterocycloalkane, cycloalkane, fused aromatic or fused heteroaromatic, all of the foregoing being unsubstituted or unsubstituted or absent.

In another preferred aspect, a novel RMA compound of the invention is identified according to the compound of Formula I, or a stereoisomer, pharmaceutically acceptable salt thereof:

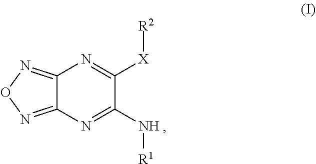

wherein,
X is —H, —OH, —S, —NH—, —NH₂, —NHCO—, —N(OH)—, or —NHOH;
R₂ is H, aryl, aryl halide, alkyl, cycloalkane, heteroalkyl, alkyl halide, aromatic, heteroaromatic, amino, amine, heterocycloalkane, cycloalkane, fused aromatic or fused heteroaromatic, all of the foregoing being substituted or unsubstituted, (CN)Ph-, (NO₂)Ph-, (CF₃)Ph-, —C(═O)R₃, —C(═)OOR₃, —S(═O)R₃, —S(═O)₂R₃, —S(═O)₂NHR₃, —C(═O)R₃, —C(═)OOR₃, —S(═O)R₃, —S(═O)₂R₃, —S(═O)₂NHR₃, —C(═O)CH₃, —OH, —(CH₂)₂OH, 2-amino-propanol, —(CH₂)₃OH, —NH₂, —NH(4-Cl)Ph, —NHNH(Ph), —NHCOCH₃, —N═C(CH₃)₂, —N═C(Ph)₂, or 3-Amino-2-propanol, —(CH₂)₂OC(═O)C₁₃H₂₇, —(CH₂)₂NHC(═O)C₁₃H₂₇, —CH₂(Ph), —(C₅H₁₀), —(CH₂)₂CH₃, —CH₃, —CH₂(2-Cl)Ph-, —(CH₂)₂CH(CH₃)₂, —(CH₂)₂(C₈H₆NCl), —(CH₂)₂(5-chloroindole), —CH₂CH(CH₃)₂—(CH₂)₂NH(C₉H₆N), —CH₂(C₃H₅NBoc), —CH₂(C₃H₅O), —CH₂(C₄H₅)F₂, —C(═O)(3-Cl)Ph-, —(CH₂)₂NH₂, —(CH₂)₂NHBoc, —(CH₂)₃NH₂, —(CH₂)₃NHBoc, —(CH₂)₂(C₈HC₈N), —NH(CH₂)₄CH₃, —CH₂(C₃H₅), —(C₄H₇), —CH₂(C₄H₇), —(C₄H₅)F₂, —C(C₃)₃, —(C₃H₅), —CH₂(3-Cl)Ph-, —CH₂(4-Cl)Ph-, —CH₂(CH)F₂, —(CH₂)₂Ph-, —(CH₂)₃NH(3-CF₃, 4, Cl) (C₅H₂N), -(4-C₁, 3-F)Ph-, —(C₁₆H₁₀), —(C₆H₃O₂)CF₂, -Fluorescein-thiourea, (2-Cl)Ph-, (4-Cl)Ph-, (2-Me)Ph-, (4-Me)Ph-, (2-OMe)Ph-, (4-OMe)Ph-, (2-OCF₃)Ph-, (4-OCF₃)Ph, (2-F)Ph-, (4-F)Ph- or absent;
R₁ is H, aryl, aryl halide, alkyl, cycloalkane, heteroalkyl, alkyl halide, aromatic, heteroaromatic, amino, amine, heterocycloalkane, cycloalkane, fused aromatic or fused heteroaromatic, all of the foregoing being substituted or unsubstituted, (CN)Ph-, (NO₂)Ph-, (CF₃)Ph-, —C(═O)R₃, —C(═)OOR₃, —S(═O)R₃, —S(═O)₂R₃, —S(═O)₂NHR₃, —C(═O)R₃, —C(═)OOR₃, —S(═O)R₃, —S(═O)₂R₃, —S(═O)₂NHR₃, —C(═O)CH₃, —OH, —(CH₂)₂OH, 2-amino-propanol, —(CH₂)₃OH, —NH₂, —NH(4-Cl)Ph, —NHNH(Ph), —NHCOCH₃, —N═C(CH₃)₂, —N═C(Ph)₂, or 3-Amino-2-propanol, —(CH₂)₂OC(═O)C₁₃H₂₇, —(CH₂)₂NHC(═O)C₁₃H₂₇, —CH₂(Ph), —(CH₁₀), —(CH₂)₂CH₃, —CH₃, —CH₂(2-Cl)Ph-, —(CH₂)₂CH(CH₃)₂, —(CH₂)₂(C₈H₆NCl), —(CH₂)₂(5-chloroindole), —CH₂CH(CH₃)₂—(CH₂)₂NH(C₉H₆N), —CH₂(C₃H₅NBoc), —CH₂(C₃H₅O), —CH₂(C₄H₅)F₂, —C(═O)(3-Cl)Ph-, —(CH₂)₂NH₂, —(CH₂)₂NHBoc, —(CH₂)₃NH₂, —(CH₂)₃NHBoc, —(CH₂)₂(C₈HC₈N), —NH(CH₂)₄CH₃, —CH₂(C₃H₅), —(C₄H₇), —CH₂(C₄H₇), —(C₄H₅)F₂, —C(C₃)₃, —(C₃H₅), —CH₂(3-Cl)Ph-, —CH₂(4-Cl)Ph-, —CH₂(CH)F₂, —(CH₂)₂Ph-, —(CH₂)₃NH(3-CF₃, 4, Cl) (C₅H₂N), -(4-Cl, 3-F)Ph-, —(C₁₆H₁₀), —(C₆H₃O₂)CF₂, -Fluorescein-thiourea, (2-Cl)Ph-, (4-Cl)Ph-, (2-Me)Ph-, (4-Me)Ph-, (2-OMe)Ph-, (4-OMe)Ph-, (2-OCF₃)Ph-, (4-OCF₃)Ph, (2-F)Ph-, (4-F)Ph- or absent; and
R₃—H, aryl, aryl halide, alkyl, alkoxy, cycloalkane, heteroalkyl, alkyl halide, aromatic, heteroaromatic, amino, amine, heterocycloalkane, cycloalkane, fused aromatic or fused heteroaromatic, all of the foregoing being substituted or unsubstituted or absent.

In one preferred aspect, R₁ of the compound according to Formula I is (C₁)Ph-, and R₂ is —(Cl)Ph-. In another preferred aspect, R₁ of the compound according to Formula I is meta (2-Cl)Ph, and R₂ is (2-Cl)Ph-. another preferred aspect, R₁ of the compound according to Formula I is para (4-Cl)Ph-, and R₂ is (4-Cl)Ph-.

In one preferred embodiment, the compound of Formula I may be used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii, and P. aeruginosa among others.

In one preferred embodiment, the compound of Formula I may be include a pharmaceutical composition that may as an antibiotic potentiator used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In another preferred embodiment, the present invention includes methods for treating a bacterial infection caused by a Gram-negative bacteria, the method comprising administering to a subject in need thereof a therapeutically effective amount of a compound of Formula I, and an antibiotic, preferably from the polymyxin family of antibiotics, wherein said compound of Formula I potentiates the activity of said antibiotic. In one embodiment, the compound of Formula I comprises a pharmaceutical composition.

In another preferred embodiment, the present invention includes methods for treating a bacterial infection caused by a Gram-negative bacteria, and preferably a mcr-positive Enterobacteriaceae and chromosomally encoded colistin-resistant A. baumannii and P. aeruginosa, the method comprising administering to a subject in need thereof a therapeutically effective amount of a compound of Formula I, and colistin, wherein said compound of Formula I potentiates the activity of said colistin. In one embodiment, the compound of Formula I comprises a pharmaceutical composition.

In another preferred embodiment, a novel RMA compound of the invention is identified according to the compound of Formula II or a stereoisomer, pharmaceutically acceptable salt thereof:

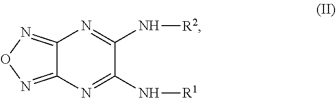

or a stereoisomer, pharmaceutically acceptable salt thereof, wherein,
R₁ is Ph-, (2-Me)Ph-, (3-Me)Ph-, (4-Me)Ph-, (2-OMe)Ph-, (3-OMe)Ph-, (4-OMe)Ph-, (2-F)Ph-, (2-F)Ph-, -(4-F)Ph-, (2-Cl)Ph-, (3-Cl)Ph-, (4-Cl)Ph-, (2-CF₃)Ph-, (3-CF₃)Ph-, (3-CF₃)Ph-, (2-OCF₃)Ph-, (3-OCF₃)Ph-, or (4-OCF₃)Ph-; and
R₂ is Ph-, (2-Me)Ph-, (3-Me)Ph-, (4-Me)Ph-, (2-OMe)Ph-, (3-OMe)Ph-, (4-OMe)Ph-, (2-F)Ph-, (2-F)Ph-, (4-F)Ph-, (2-Cl)Ph-, (3-Cl)Ph-, (4-Cl)Ph-, (2-CF₃)Ph-, (3-CF₃)Ph-, (3-CF₃)Ph-, (2-OCF₃)Ph-, (3-OCF₃)Ph-, or (4-OCF₃)Ph-.

In one preferred aspect, R₁ of the compound according to Formula II is (2-Cl)Ph-, and R₂ is (2-Cl)Ph-. In one preferred aspect, R₁ of the compound according to Formula II is (4-Cl)Ph-, and R₂ is (4-Cl)Ph-.

In one preferred embodiment, the compound of Formula III may be used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In one preferred embodiment, the compound of Formula II may be include a pharmaceutical composition that may as an antibiotic potentiator used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In another preferred embodiment, the present invention includes methods for treating a bacterial infection caused by a Gram-negative bacteria, the method comprising administering to a subject in need thereof a therapeutically effective amount of a compound of Formula II, and an antibiotic, preferably from the polymyxin family of antibiotics, wherein said compound of Formula II potentiates the activity of said antibiotic. In one embodiment, the compound of Formula II comprises a pharmaceutical composition.

In another preferred embodiment, the present invention includes methods for treating a bacterial infection caused by a Gram-negative bacteria, and preferably a mcr-positive Enterobacteriaceae and chromosomally encoded colistin-resistant A. baumannii and P. aeruginosa, the method comprising administering to a subject in need thereof a therapeutically effective amount of a compound of Formula II, and colistin, wherein said compound of Formula II potentiates the activity of said colistin. In one embodiment, the compound of Formula II comprises a pharmaceutical composition.

In another preferred embodiment, a novel RMA compound of the invention is identified according to the compound of Formula VII or a stereoisomer, pharmaceutically acceptable salt thereof:

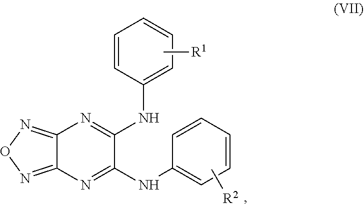

wherein
R₁ is —H, —Br, —CN, —NO₂, —CF₃, —Cl, -Me, —OMe, —F, or —OCF₃; and
R₂ is —H, —Br, —CN, —NO₂, —CF₃, —Cl, -Me, —OMe, —F, or —OCF₃.

In one preferred aspect, R₁ of the compound according to Formula VII is Cl, and R₂ is Cl. In one preferred aspect, R₁ of the compound according to Formula VII is p-Cl, and R₂ is p-Cl. In one preferred aspect, R₁ of the compound according to Formula VII is m-Cl, and R₂ is m-Cl.

In one preferred embodiment, the compound of Formula VII may be used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In one preferred embodiment, the compound of Formula VII may be include a pharmaceutical composition that may as an antibiotic potentiator used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In another preferred embodiment, the present invention includes methods for treating a bacterial infection caused by a Gram-negative bacteria, the method comprising administering to a subject in need thereof a therapeutically effective amount of a compound of Formula VII, and an antibiotic, preferably from the polymyxin family of antibiotics, wherein said compound of Formula VII potentiates the activity of said antibiotic. In one embodiment, the compound of Formula VII comprises a pharmaceutical composition.

In another preferred embodiment, the present invention includes methods for treating a bacterial infection caused by a Gram-negative bacteria, and preferably a mcr-positive Enterobacteriaceae and chromosomally encoded colistin-resistant A. baumannii and P. aeruginosa, the method comprising administering to a subject in need thereof a therapeutically effective amount of a compound of Formula III, and colistin, wherein said compound of Formula VII potentiates the activity of said colistin. In one embodiment, the compound of Formula VII comprises a pharmaceutical composition.

In another preferred embodiment, a novel RMA compound of the invention is identified according to the compound of Formula VIII or XI or a stereoisomer, pharmaceutically acceptable salt thereof:

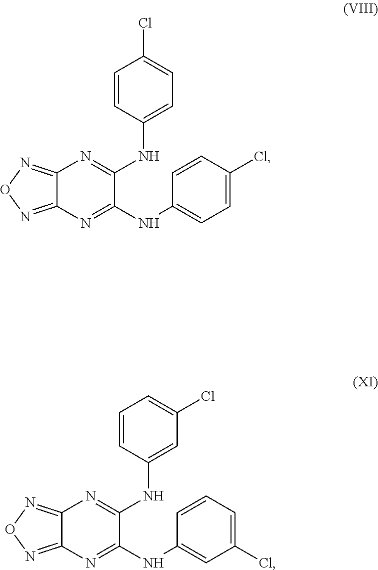 [SMILES: Clc1ccc(Nc2nc3nonc3nc2Nc2ccc(Cl)cc2)cc1 ; Clc1cccc(Nc2nc3nonc3nc2Nc2cccc(Cl)c2)c1]

In one preferred embodiment, the compound of Formula VIII or XI may be used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In one preferred embodiment, the compound of Formula VIII or XI may be include a pharmaceutical composition that may as an antibiotic potentiator used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In another preferred embodiment, the present invention includes methods for treating a bacterial infection caused by a Gram-negative bacteria, the method comprising administering to a subject in need thereof a therapeutically effective amount of a compound of Formula VIII or XI, and an antibiotic, preferably from the polymyxin family of antibiotics, wherein said compound of Formula VIII or XI potentiates the activity of said antibiotic. In one embodiment, the compound of Formula VIII or XI comprises a pharmaceutical composition.

In another preferred embodiment, the present invention includes methods for treating a bacterial infection caused by a Gram-negative bacteria, and preferably a mcr-positive Enterobacteriaceae and chromosomally encoded colistin-resistant A. baumannii and P. aeruginosa, the method comprising administering to a subject in need thereof a therapeutically effective amount of a compound of Formula VIII or XI, and colistin, wherein said compound of Formula VIII or XI potentiates the activity of said colistin. In one embodiment, the compound of Formula VIII or XI comprises a pharmaceutical composition.

In another preferred embodiment, a novel RMA compound of the invention is identified according to the compound of Formula III, or a stereoisomer, pharmaceutically acceptable salt thereof

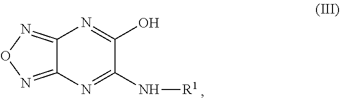

wherein,
R₁ is Ph-, (2-Me)Ph-, (3-Me)Ph-, (4-Me)Ph-, (2-OMe)Ph-, (3-OMe)Ph-, (4-OMe)Ph-, (2-Cl)Ph-, (3-Cl)Ph-, (4-Cl)Ph-, (2-F)Ph-, (3-F)Ph-, (4-F)Ph-, (2,3-Cl)Ph-, (2-CF₃)Ph-, (3-CF₃)Ph-, (2-OCF₃)Ph-, (3-OCF₃)Ph-, (4-OCF₃)Ph-, substituted or unsubstituted heteroaromatic,

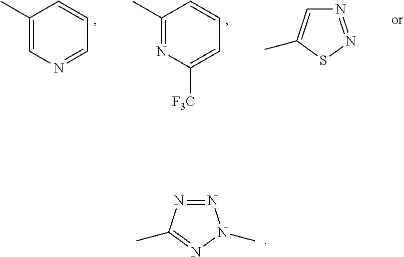

In one preferred embodiment, the compound of Formula III may be used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In one preferred embodiment, the compound of Formula III may be include a pharmaceutical composition that may as an antibiotic potentiator used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In another preferred embodiment, the present invention includes methods for treating a bacterial infection caused by a Gram-negative bacteria, the method comprising administering to a subject in need thereof a therapeutically effective amount of a compound of Formula III, and an antibiotic, preferably from the polymyxin family of antibiotics, wherein said compound of Formula III potentiates the activity of said antibiotic. In one embodiment, the compound of Formula III comprises a pharmaceutical composition.

In another preferred embodiment, the present invention includes methods for treating a bacterial infection caused by a Gram-negative bacteria, and preferably a mcr-positive Enterobacteriaceae and chromosomally encoded colistin-resistant A. baumannii and P. aeruginosa, the method comprising administering to a subject in need thereof a therapeutically effective amount of a compound of Formula III, and colistin, wherein said compound of Formula III potentiates the activity of said colistin. In one embodiment, the compound of Formula III comprises a pharmaceutical composition.

In another preferred embodiment, a novel RMA compound of the invention is identified according to the compound of Formula IV, or a stereoisomer, pharmaceutically acceptable salt thereof:

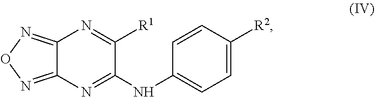

wherein,
R₁ is —NH₂, —NHOH, —NH(CH₂)₂OH, or —NHCOCH₃,
R₂ is —OCF₃; or
In one preferred embodiment, the compound of Formula IV may be used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In one preferred embodiment, the compound of Formula IV may be include a pharmaceutical composition that may as an antibiotic potentiator used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In another preferred embodiment, the present invention includes methods for treating a bacterial infection caused by a Gram-negative bacteria, the method comprising administering to a subject in need thereof a therapeutically effective amount of a compound of Formula IV, and an antibiotic, preferably from the polymyxin family of antibiotics, wherein said compound of Formula IV potentiates the activity of said antibiotic. In one embodiment, the compound of Formula IV comprises a pharmaceutical composition.

In another preferred embodiment, the present invention includes methods for treating a bacterial infection caused by a Gram-negative bacteria, and preferably a mcr-positive Enterobacteriaceae and chromosomally encoded colistin-resistant A. baumannii and P. aeruginosa, the method comprising administering to a subject in need thereof a therapeutically effective amount of a compound of Formula IV, and colistin, wherein said compound of Formula IV potentiates the activity of said colistin. In one embodiment, the compound of Formula IV comprises a pharmaceutical composition.

In another preferred embodiment, a novel RMA compound of the invention is identified according to the compound of Formula V, or a stereoisomer, pharmaceutically acceptable salt thereof:

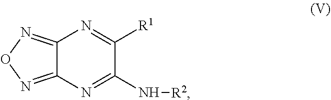

wherein,
R₁ is —NH(CH₂)₂OH, 2-amino-propanol, —NH(CH₂)₃OH, —NHNH₂, —NHNH(4-Cl)Ph, —NHNH(Ph), —NHNHCOCH₃, —NHN═C(CH₃)₂, —NHN═C(Ph)₂, —NHCH₂CH(CH₃OH), —NH(CH₂)₂OCO(CH₂)₁₂CH₃, —NH(CH₂)₂NHCO(CH₂)₁₂CH₃, —NHCH₂(Ph), or —NHCO(Ph); and R₂ is —(CH₂)Ph, (2-OCF₃)Ph-, —(3-OCF₃)Ph, —NHC═O(Ph).

In one preferred embodiment, the compound of Formula V may be used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In one preferred embodiment, the compound of Formula V may be include a pharmaceutical composition that may as an antibiotic potentiator used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In another preferred embodiment, the present invention includes methods for treating a bacterial infection caused by a Gram-negative bacteria, the method comprising administering to a subject in need thereof a therapeutically effective amount of a compound of Formula V, and an antibiotic, preferably from the polymyxin family of antibiotics, wherein said compound of Formula V potentiates the activity of said antibiotic. In one embodiment, the compound of Formula V comprises a pharmaceutical composition.

In another preferred embodiment, the present invention includes methods for treating a bacterial infection caused by a Gram-negative bacteria, and preferably a mcr-positive Enterobacteriaceae and chromosomally encoded colistin-resistant A. baumannii and P. aeruginosa, the method comprising administering to a subject in need thereof a therapeutically effective amount of a compound of Formula V, and colistin, wherein said compound of Formula V potentiates the activity of said colistin. In one embodiment, the compound of Formula V comprises a pharmaceutical composition.

In another preferred embodiment, a novel RMA compound of the invention is identified according to the compound of Formula VI, or a stereoisomer, pharmaceutically acceptable salt thereof:

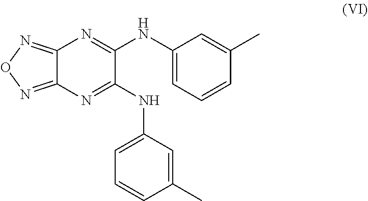 [SMILES: Cc1cccc(Nc2nc3nonc3nc2Nc2cccc(C)c2)c1]

In one preferred embodiment, the compound of Formula VI may be used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In one preferred embodiment, the compound of Formula VI may be include a pharmaceutical composition that may as an antibiotic potentiator used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In another preferred embodiment, the present invention includes methods for treating a bacterial infection caused by a Gram-negative bacteria, the method comprising administering to a subject in need thereof a therapeutically effective amount of a compound of Formula VI, and an antibiotic, preferably from the polymyxin family of antibiotics, wherein said compound of Formula VI potentiates the activity of said antibiotic. In one embodiment, the compound of Formula VI comprises a pharmaceutical composition.

In another preferred embodiment, the present invention includes methods for treating a bacterial infection caused by a Gram-negative bacteria, and preferably a mcr-positive Enterobacteriaceae and chromosomally encoded colistin-resistant A. baumannii and P. aeruginosa, the method comprising administering to a subject in need thereof a therapeutically effective amount of a compound of Formula VI, and colistin, wherein said compound of Formula VI potentiates the activity of said colistin. In one embodiment, the compound of Formula VI comprises a pharmaceutical composition.

In another preferred embodiment, a novel RMA compound of the invention is identified according to the compound of Formula X, or a stereoisomer, pharmaceutically acceptable salt thereof:

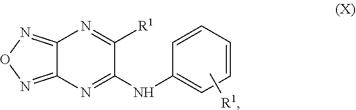

wherein R₁ is —H, —Cl, -Me, —OMe, —F, —OCF₃.

In one preferred embodiment, the compound of Formula X may be used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In one preferred embodiment, the compound of Formula X may be include a pharmaceutical composition that may as an antibiotic potentiator used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In another preferred embodiment, the present invention includes methods for treating a bacterial infection caused by a Gram-negative bacteria, the method comprising administering to a subject in need thereof a therapeutically effective amount of a compound of Formula X, and an antibiotic, preferably from the polymyxin family of antibiotics, wherein said compound of Formula X potentiates the activity of said antibiotic. In one embodiment, the compound of Formula X comprises a pharmaceutical composition.

In another preferred embodiment, the present invention includes methods for treating a bacterial infection caused by a Gram-negative bacteria, and preferably a mcr-positive Enterobacteriaceae and chromosomally encoded colistin-resistant A. baumannii and P. aeruginosa, the method comprising administering to a subject in need thereof a therapeutically effective amount of a compound of Formula X, and colistin, wherein said compound of Formula X potentiates the activity of said colistin. In one embodiment, the compound of Formula X comprises a pharmaceutical composition.

In another preferred embodiment, a novel RMA compound of the invention is identified according to the compound of Formula II, or a stereoisomer, pharmaceutically acceptable salt thereof:

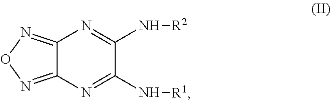

wherein,
R₁ and R₂ are substituted or unsubstituted aromatic, wherein said substituted aromatic preferably comprises a substituted phenyl, and wherein said substituted phenyl preferably comprises m-Cl or p-Cl.

In another preferred embodiment, a novel RMA compound of the invention comprises a compound selected from the group consisting of (the below group, and the compounds of Formulas I-XI, being generally referred to as the/a compound of the invention, or a RMA compound of the invention):

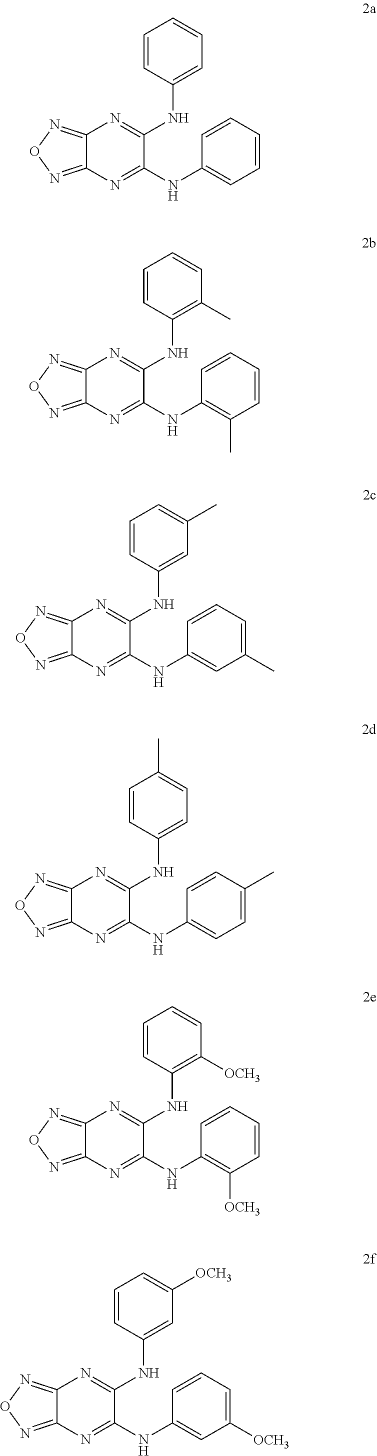 [SMILES: c1ccc(Nc2nc3nonc3nc2Nc2ccccc2)cc1 ; Cc1ccccc1Nc1nc2nonc2nc1Nc1ccccc1C ; Cc1cccc(Nc2nc3nonc3nc2Nc2cccc(C)c2)c1 ; Cc1ccc(Nc2nc3nonc3nc2Nc2ccc(C)cc2)cc1 ; COc1ccccc1Nc1nc2nonc2nc1Nc1ccccc1OC ; COc1cccc(Nc2nc3nonc3nc2Nc2cccc(OC)c2)c1]

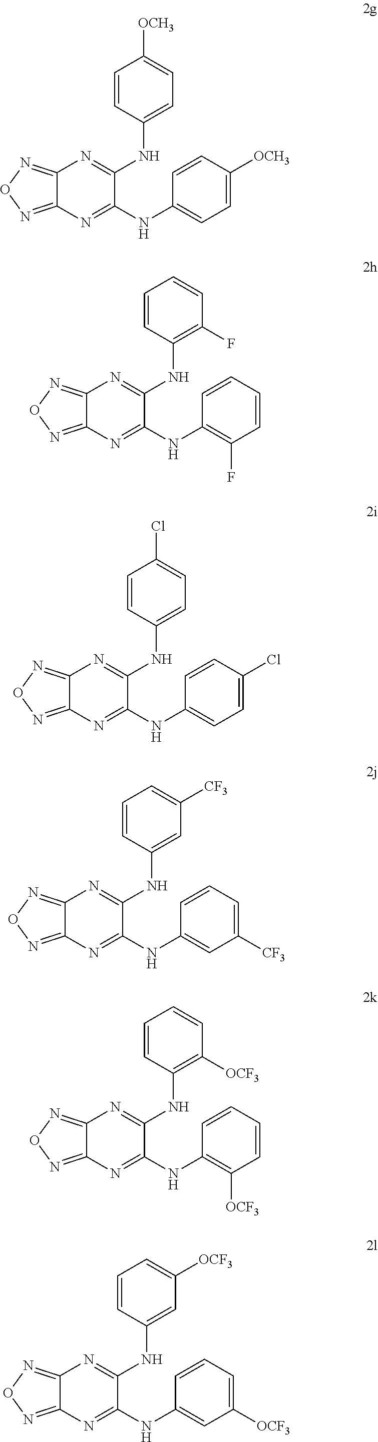 [SMILES: COc1ccc(Nc2nc3nonc3nc2Nc2ccc(OC)cc2)cc1 ; Fc1ccccc1Nc1nc2nonc2nc1Nc1ccccc1F ; Clc1ccc(Nc2nc3nonc3nc2Nc2ccc(Cl)cc2)cc1 ; FC(F)(F)c1cccc(Nc2nc3nonc3nc2Nc2cccc(C(F)(F)F)c2)c1 ; FC(F)(F)Oc1ccccc1Nc1nc2nonc2nc1Nc1ccccc1OC(F)(F)F ; FC(F)(F)Oc1cccc(Nc2nc3nonc3nc2Nc2cccc(OC(F)(F)F)c2)c1]

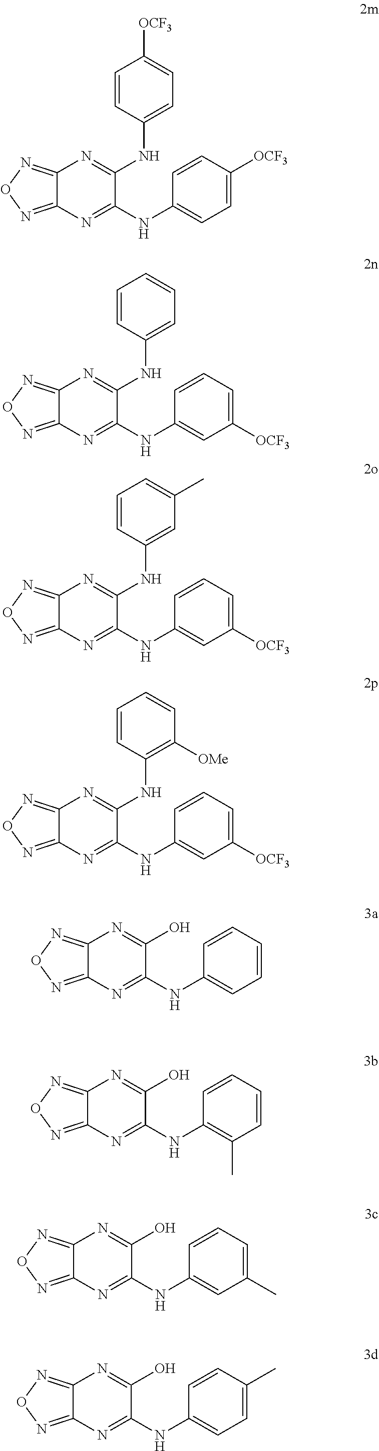

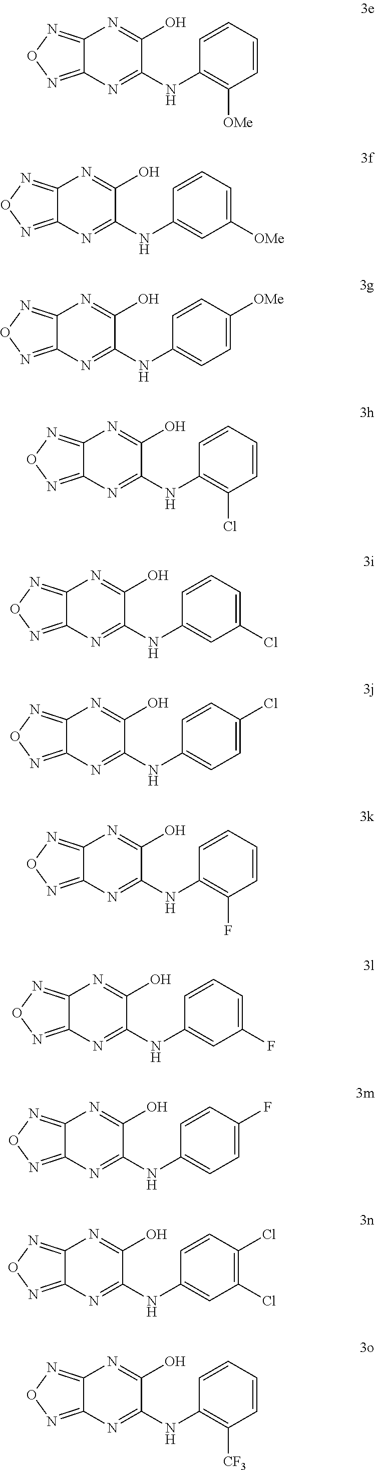

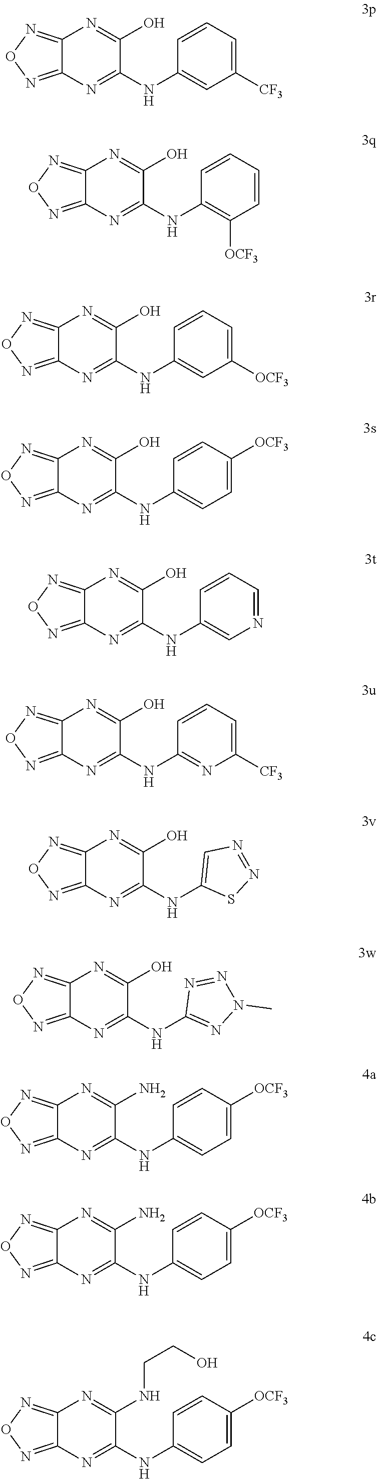

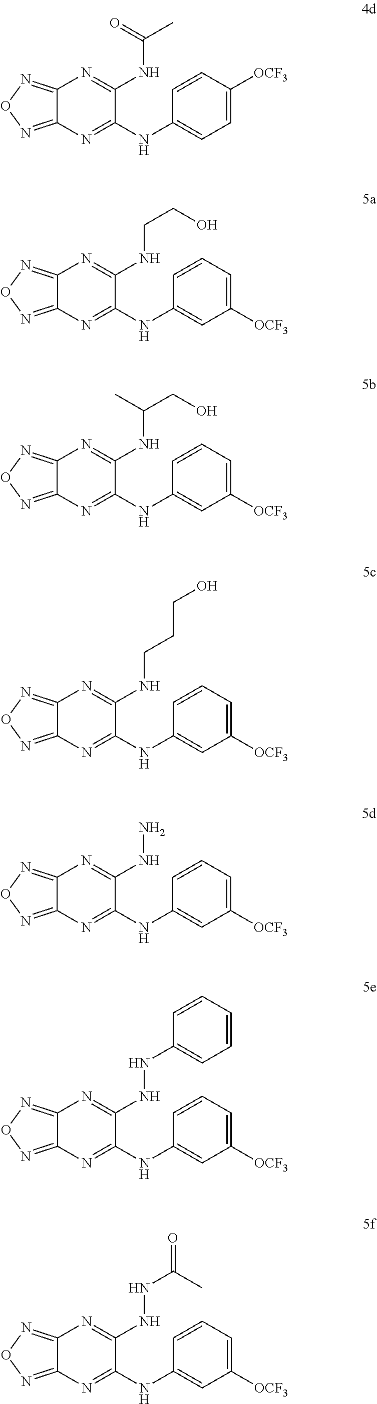

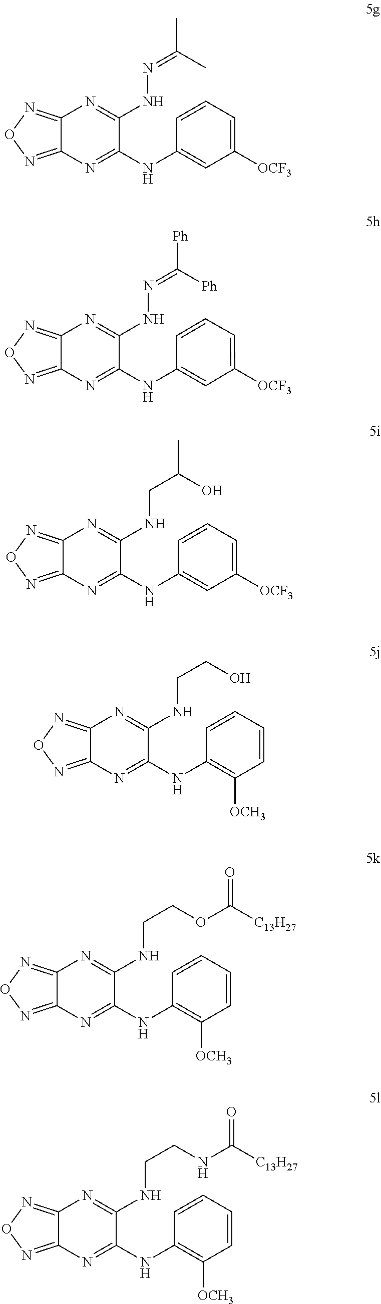

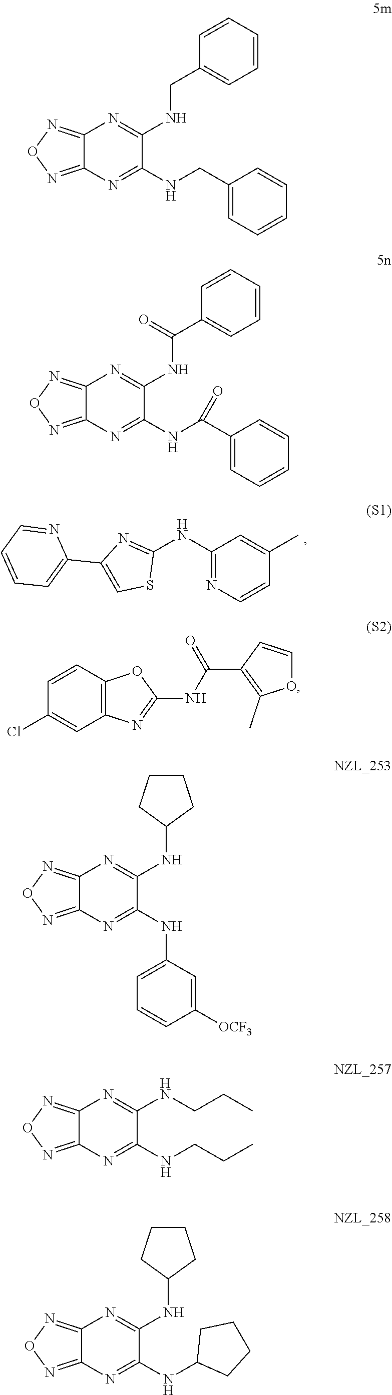

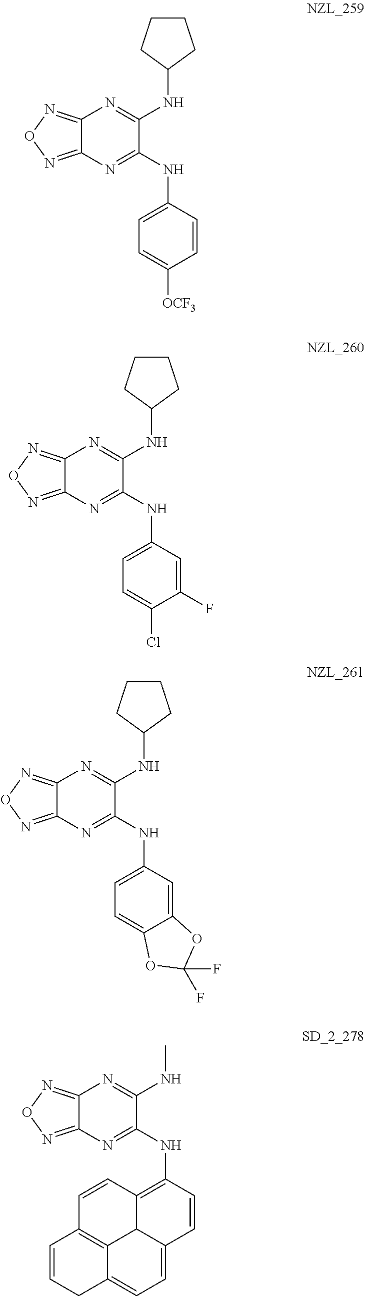

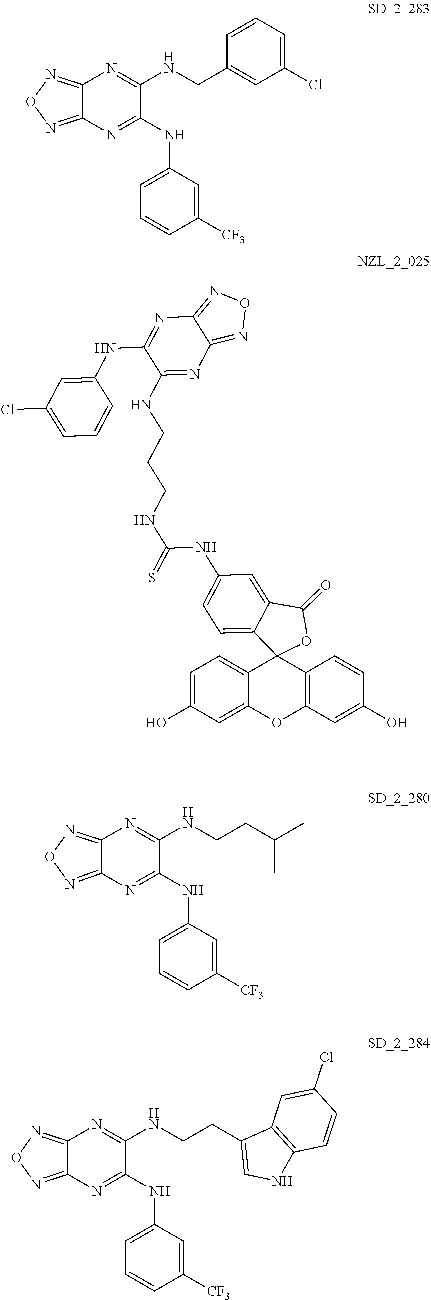

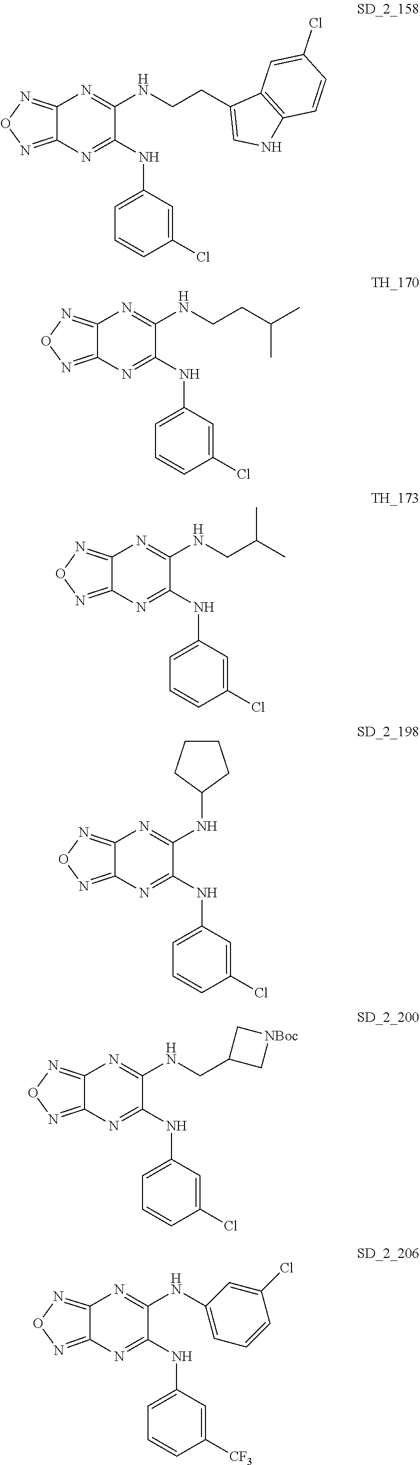

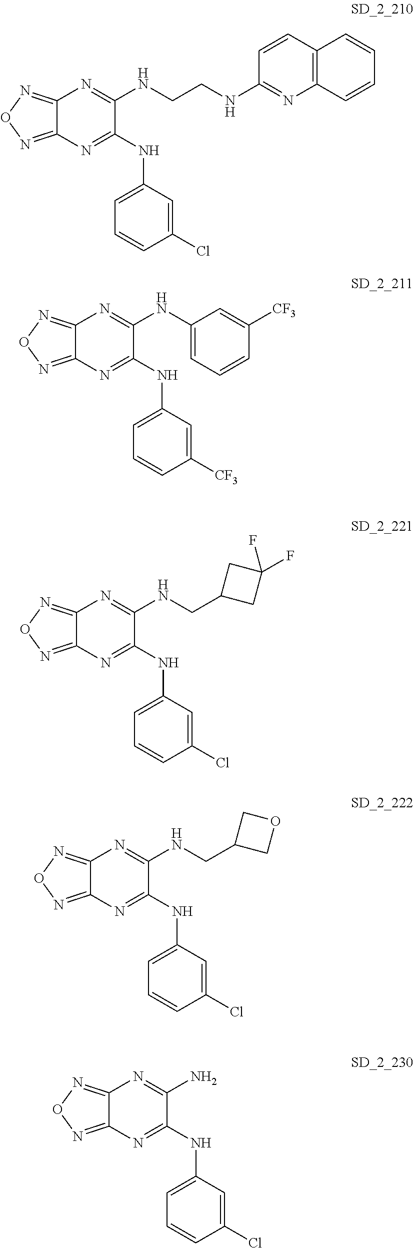 [SMILES: Clc1cccc(Nc2nc3nonc3nc2NCCNc2ccc3ccccc3n2)c1 ; FC(F)(F)c1cccc(Nc2nc3nonc3nc2Nc2cccc(C(F)(F)F)c2)c1 ; FC1(F)CC(CNc2nc3nonc3nc2Nc2cccc(Cl)c2)C1 ; Clc1cccc(Nc2nc3nonc3nc2NCC2COC2)c1 ; Nc1nc2nonc2nc1Nc1cccc(Cl)c1]

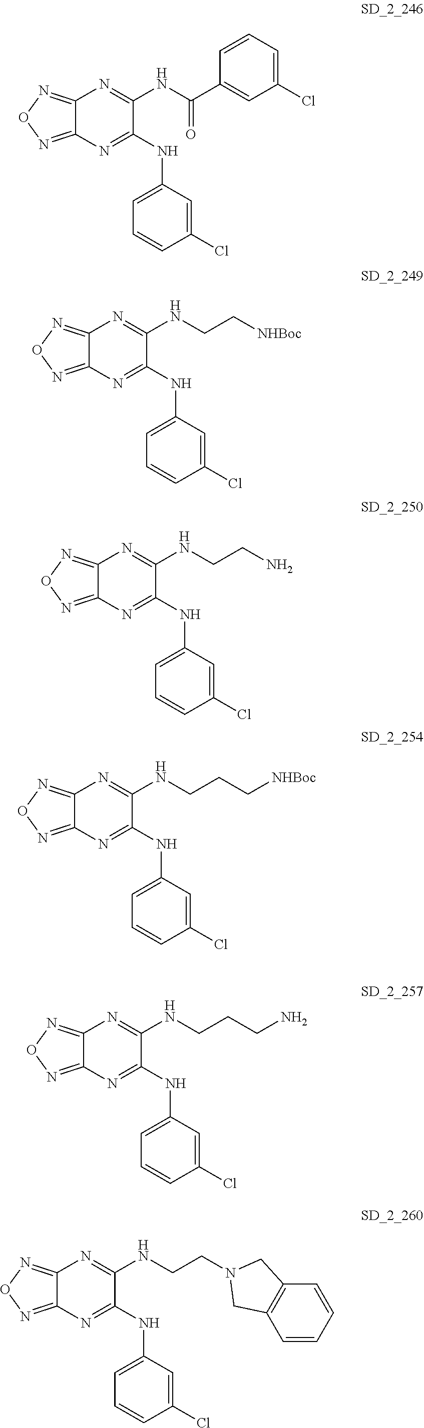 [SMILES: O=C(Nc1nc2nonc2nc1Nc1cccc(Cl)c1)c1cccc(Cl)c1 ; CC(C)(C)OC(=O)NCCNc1nc2nonc2nc1Nc1cccc(Cl)c1 ; NCCNc1nc2nonc2nc1Nc1cccc(Cl)c1 ; CC(C)(C)OC(=O)NCCCNc1nc2nonc2nc1Nc1cccc(Cl)c1 ; NCCCNc1nc2nonc2nc1Nc1cccc(Cl)c1 ; Clc1cccc(Nc2nc3nonc3nc2NCCN2Cc3ccccc3C2)c1]

or a stereoisomer, pharmaceutically acceptable salt thereof.

In one preferred embodiment, one or more of compounds of the invention may be used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In one preferred embodiment, one or more of compounds of the invention may be include a pharmaceutical composition that may as an antibiotic potentiator used in combination with one or more antibiotics, such as the polymyxin family antibiotics, and in preferably colistin, as an antibiotic potentiator to treat antibiotic resistant bacterial infections such as those caused by Gram-negative bacteria, and in particular mcr-positive Enterobacteriaceae and chromosomally encoded colistin-resistant A. baumannii and P. aeruginosa among others.

In another preferred embodiment, the present invention includes methods for treating a bacterial infection caused by a Gram-negative bacteria, the method comprising administering to a subject in need thereof a therapeutically effective amount of a compound selected from the group consisting of one or more compounds of the invention, and an antibiotic, preferably from the polymyxin family of antibiotics, wherein said compound potentiates the activity of said antibiotic. In one embodiment, the compound comprises a pharmaceutical composition.

In another preferred embodiment, the present invention includes methods for treating a bacterial infection caused by a Gram-negative bacteria, and preferably a mcr-positive Enterobacteriaceae and chromosomally encoded colistin-resistant A. baumannii and P. aeruginosa, the method comprising administering to a subject in need thereof a therapeutically effective amount of a compound selected from the group consisting of one or more compounds of the invention, and colistin, wherein said compound potentiates the activity of said colistin. In one embodiment, the compound comprises a pharmaceutical composition.

Another aspect of the invention provides an antibiotic composition comprising a compound of the invention that is capable of re-sensitizing the susceptibility of a resistant bacteria, such as a MDR gram-negative bacteria to a polymyxin family of antibiotics, and preferably colistin or polymyxin B. Suitable polymyxin antibiotics are well known to one skilled in the art, and exemplary polymyxin antibiotics can be found in Merck Index, 15th Ed., Edited by Maryadele J O'Neil, Royal Society of Chemistry, 2013, and Physicians' Desk Reference (i.e., “PDR”) 67thEd., 2013, all of which are incorporated herein by reference in their entirety.

The term “gram-negative bacteria” as used herein refers to bacteria stained red by Gram staining and, generally, these bacteria have strong resistance to pigments and surfactants. The gram-negative bacteria of the present invention include all types of gram-negative bacteria containing endotoxins, and examples thereof include, but are not limited to, bacteria belonging to the genus Escherichia, the genus Pseudomonas, the genus Acinetobacter, the genus Salmonella, the genus Klebsiella, the genus Neisseria, the genus Enterobacter, the genus Shigella, the genus Moraxella, the genus Helicobacter, the genus Stenotrophomonas, the genus Bdellovibrio, and the genus Legionella. In particular, examples of these gram-negative bacteria include, but are not limited to, Escherichia coli, Pseudomonas aeruginosa, Pseudomonas fluorescens, Pseudomonas putida, Pseudomonas chlororaphis, Pseudomonas pertucinogena, Pseudomonas stutzeri, Pseudomonas syringae, Acinetobacter baumannii, Acinetobacter lwoffii, Acinetobacter calcoaceticus, Acinetobacter haemolyticus, Salmonella enterica, Salmonella bongori, Salmonella enteritidis, Salmonella typhimurium, Salmonella gallinarum, Salmonella pullorum, Salmonella mbandaka, Salmonella choleraesuls, Salmonella thompson, Salmonella infantis, Salmonella derby, Klebsiella pneumonia, Klebsiella granulomatis, Klebsiella oxytoca, Klebsiella terrigena, Neisseria gonorrhoeae, Neisseria meningitidis, Enterobacter aerogenes, Enterobacter cloacae, Shigella boydii, Shigella dysenteriae, Shigella flexneri, Shigella sonnei, Moraxella catarrhalis, Moraxella lacunata, Moraxella bovis, Helicobacter pylori, Helicobacter heilmannii, Helicobacter felis, Helicobacter mustelae, Helicobacter fenelliae, Helicobacter rappini, Helicobacter hepaticus, Helicobacter bilis, Helicobacter pullorum, Stenotrophomonas maltophilia, Stenotrophomonas nitritireducens, Bdellovibrio bacteriovorus, Legionella pneumophila, Legionella anisa, Legionella birminghamensis, Legionella bozemanii, Legionella cincinnatiensis, Legionella dumoffii, Legionella feeleii, Legionella gormanii, Legionella hackeliae, Legionella israelensis, Legionella jordanis, Legionella lansingensis, Legionella longbeachae, Legionella maceachernii, Legionella micdadei, Legionella oakridgensis, Legionella sainthelensi, Legionella tucsonensis, and Legionella wadsworthii.

Within the meaning of the present invention polymyxin means antibiotic with a chemical structure of a cyclic peptide with a hydrophobic tail; polymyxins include polymyxin B and polymyxin E, known as colistin, pharmaceutically acceptable salts thereof, or a mixture thereof. Within the meaning of the present invention multidrug resistant bacterial pathogens means any bacteria which are resistant to multiple antimicrobial drugs and in particular antibiotics, in particular Gram-negative species. Within the meaning of the present invention a polymyxin-resistant Gram-negative bacteria means any bacterial being resistant to polymyxin in particular Gram-negative bacteria such as E. coli, S. maltophilia, E. cloacae, K. pneumoniae, A. baumannii, Salmonella and P. aeruginosa. In many cases, a polymyxin-resistant Gram-negative may express obtain resistance to polymyxin via Mobilized Colistin Resistance (mcr) plasmids.

The compounds of the invention can be administered to a patient or a subject to achieve a desired physiological effect. Generally, the subject is an animal, typically a mammal, and preferably a human. The compound can be administered in a variety of forms adapted to the chosen route of administration, i.e., orally, or parenterally. Parenteral administration in this respect includes administration by the following routes: intravenous; intramuscular; subcutaneous; intraocular; intrasynovial; transepithelially including transdermal, ophthalmic, sublingual and buccal; topically including ophthalmic, dermal, ocular, rectal and nasal inhalation via insufflation and aerosol; intraperitoneal; and rectal systemic.

One or more compounds of the invention may include a pharmaceutical composition. In alternative embodiment, the pharmaceutical composition of the invention may include a compound of the invention and at least one antibiotic, such as a polymyxin family of antibiotic, such as preferably colistin.

A “pharmaceutical composition” or “pharmaceutical composition of the invention” refers to a compound of the invention or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients. In other embodiments, the pharmaceutical composition further comprises at least one additional antibiotic, such as through a co-treatment. As used herein, a “pharmaceutically acceptable carrier” refers to a carrier or diluent that does not cause significant irritation to an organism and does not abrogate the biological activity and properties of the administered composition of the invention. The pharmaceutical acceptable carrier may comprise any conventional pharmaceutical carrier or excipient. The choice of carrier and/or excipient will to a large extent depend on factors such as the particular mode of administration, the effect of the carrier or excipient on solubility and stability, and the nature of the dosage form.

Suitable pharmaceutical carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin, and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension, or emulsion, for topical administration as an ointment or cream or for rectal administration as a suppository. The pharmaceutical composition may be in unit dosage forms suitable for single administration of precise dosages. Exemplary parenteral administration forms include solutions or suspensions of active compounds in sterile aqueous solutions, for example, aqueous propylene glycol or dextrose solutions. Such dosage forms may be suitably buffered, if desired.

A pharmaceutical composition of the invention may be administered as single agents, for example a pharmaceutical composition of a compound of the invention, or a pharmaceutical composition of a compound of the invention, or may be administered in combination with an antibiotic, and preferably polymyxin family of antibiotic, such as polymyxin A or colistin. In some embodiments, the methods provided result in one or more of the following effects: (1) treating a MDR gram-negative bacterial infection in a subject, and preferably a polymyxin resistant Gram-negative bacteria; (2) inhibiting growth of gram-negative bacteria; (3) preventing infection of a MDR gram-negative bacterial infection in a subject; and (4) sensitizing or re-sensitizing a MDR gram-negative bacterial infection to an antibiotic and preferably polymyxin family of antibiotic, such as polymyxin A or colistin. Pharmaceutical compositions suitable for the delivery of one or more compounds of the invention as described herein, and methods for their preparation will be readily apparent to those skilled in the art. Such compositions and methods for their preparation can be found, for example, in ‘Remington's Pharmaceutical Sciences’, 19th Edition (Mack Publishing Company, 1995), the disclosure of which is incorporated herein by reference in its entirety.

The active compound can be orally administered, for example, with an inert diluent or with an assimilable edible carrier, or it can be enclosed in hard or soft shell gelatin capsules, or it can be compressed into tablets, or it can be incorporated directly with the food of the diet. For oral therapeutic administration, the active compound may be incorporated with excipient and used in the form of ingestible tablets, buccal tablets, troches, capsules, elixirs, suspensions, syrups, wafers, and the like. Such compositions and preparation can contain at least 0.1% of active compound. The percentage of the compositions and preparation can, of course, be varied and can conveniently be between about 1 to about 10% of the weight of the unit. The amount of active compound in such therapeutically useful compositions is such that a suitable dosage will be obtained. Typical compositions or preparations according to the invention are prepared such that an oral dosage unit form contains from about 1 to about 1000 mg of active compound.

The tablets, troches, pills, capsules and the like can also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin can be added or a flavoring agent such as peppermint, oil of wintergreen, or cherry flavoring. When the dosage unit form is a capsule, it can contain, in addition to materials of the above type, a liquid carrier. Various other materials can be present as coatings or to otherwise modify the physical form of the dosage unit. For instance, tablets, pills, or capsules can be coated with shellac, sugar or both. A syrup or elixir can contain the active compound, sucrose as a sweetening agent, methyl and propylparabens as preservatives, a dye and flavoring such as cherry or orange flavor. Of course, any material used in preparing any dosage unit form should be pharmaceutically pure and substantially non-toxic in the amounts employed. In addition, the active compound can be incorporated into sustained-release preparations and formulation. In addition to the common dosage forms set out above, the compounds of the invention may also be administered by controlled release means and/or delivery devices capable of releasing the active ingredient (prenylation inhibitor) at the required rate to maintain constant pharmacological activity for a desirable period of time. Such dosage forms provide a supply of a drug to the body during a predetermined period of time and thus maintain drug levels in the therapeutic range for longer periods of time than conventional non-controlled formulations. Examples of controlled release pharmaceutical compositions and delivery devices that may be adapted for the administration of the active ingredients of the present invention are described in U.S. Pat. Nos. 3,847,770; 3,916,899; 3,536,809; 3,598,123; 3,630,200; 4,008,719; 4,687,610; 4,769,027; 5,674,533; 5,059,595; 5,591,767; 5,120,548; 5,073,543; 5,639,476; 5,354,566; and 5,733,566, the disclosures of which are hereby incorporated by reference.

Pharmaceutical compositions for use in the methods of the present invention may be prepared by any of the methods of pharmacy, but all methods include the step of bringing the active ingredient into association with the carrier which constitutes one or more necessary ingredients. In general, the compositions are prepared by uniformly and intimately admixing the active ingredient with liquid carriers or finely divided solid carriers or both, and then, if necessary, shaping the product into the desired presentation.

For example, a tablet may be prepared by compression or molding, optionally with one or more accessory ingredients. Compressed tablets may be prepared by compressing in a suitable machine the active ingredient in a free-flowing form such as powder or granules, optionally mixed with a binder, lubricant, inert diluent, surface active or dispersing agent. Molded tablets may be made by molding, in a suitable machine, a mixture of the powdered compound moistened with an inert liquid diluent.

The compound of the invention can also be administered parenterally. Solutions of the active compound as a free base or pharmacologically acceptable salt can be prepared in water suitably mixed with a surfactant such as hydroxypropylcellulose. Dispersion can also be prepared in glycerol, liquid polyethylene glycols, and mixtures thereof and in oils. Under ordinary conditions of storage and use, these preparations contain a preservative to prevent the growth of microorganisms.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringability exists. It can be stable under the conditions of manufacture and storage and must be preserved against the contaminating action of microorganisms such as bacterial and fungi. The carrier can be a solvent of dispersion medium containing, for example, water, ethanol, polyol (e.g., glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, e.g., sugars or sodium chloride. Prolonged absorption of the injectable compositions of agents delaying absorption, e.g., aluminum monostearate and gelatin.

Sterile injectable solutions are prepared by incorporating the compound of the invention in the required amount in the appropriate solvent with various other ingredients enumerated above, as required, followed by filtered sterilization. Generally, dispersions are prepared by incorporating the various sterilized ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique which yield a powder of the active ingredient plus any additional desired ingredient from previously sterile-filtered solution thereof.

The compounds of the invention can be administered to a mammal alone or in combination with pharmaceutically acceptable carriers, as noted above, the proportion of which is determined by the solubility and chemical nature of the compound, chosen route of administration and standard pharmaceutical practice.

The physician can readily determine the dosage of the present therapeutic agents which will be most suitable for prophylaxis or treatment and it will vary with the form of administration and the particular compound chosen, and also, it will vary with the particular patient under treatment. The physician will generally wish to initiate treatment with small dosages by small increments until the optimum effect under the circumstances is reached. The therapeutic dosage can generally be from about 0.1 to about 1000 mg/day, and preferably from about 10 to about 100 mg/day, or from about 0.1 to about 50 mg/Kg of body weight per day and preferably from about 0.1 to about 20 mg/Kg of body weight per day and can be administered in several different dosage units. Higher dosages, on the order of about 2× to about 4×, may be required for oral administration.

The term “aniline” means an organic compound with the formula C₆H₅NH₂. Consisting of a phenyl group attached to an amino group, which may include one or more additional substituent R-groups.

Terms “halide,” “halogen” and “halo” are used interchangeably herein and refer to fluoro, chloro, bromo, or iodo.

The term “alkyl” refers to a saturated linear monovalent hydrocarbon moiety of one to twenty, typically one to fifteen, and often one to ten carbon atoms or a saturated branched monovalent hydrocarbon moiety of three to twenty, typically three to fifteen, and often three to ten carbon atoms. Exemplary alkyl group include, but are not limited to, methyl, ethyl, n-propyl, 2-propyl, tert-butyl, pentyl, iso-pentyl, hexyl, and the like.

As used herein, the term “protecting group” means a group which has been introduced onto a functional group in a compound and which modifies the said functional group's chemical reactivity. In one embodiment, a protecting group comprises a Boc protecting group. The term “Boc” refers to tert-butyloxycarbonyl.

“Alkylene” refers to a saturated linear divalent hydrocarbon moiety of one to twenty, typically one to fifteen and often one to ten carbon atoms or a branched saturated divalent hydrocarbon moiety of three to twenty, typically three to fifteen and often three to ten carbon atoms. Exemplary alkylene groups include, but are not limited to, methylene, ethylene, propylene, butylene, pentylene, and the like.

The term “alkyl” means a straight chain, branched chain, or cyclic alkyl group such as the examples given above, as well as dodecyl, tetradecyl, pentadecyl, hexadecyl, heptadecyl, octadecyl, nonadecyl, and icosyl group. These alkyl groups may be substituted with various substituents. Examples of these substituents are the same as those given above for “alkyl groups with 1 to 12 carbon atoms”.

“Haloalkyl” or “alkyl halide” refers to an alkyl group as defined herein in which one or more hydrogen atom is replaced by same or different halide atoms. Exemplary haloalkyls include, but are not limited to, —CH₂Cl, —CF₃, —CH₂CF₃, —CH₂CCl₃, and the like.

“Aryl” refers to a monovalent mono-, bi- or tricyclic aromatic hydrocarbon moiety of 6 to 15 ring atoms such as phenyl, naphthyl, etc. Aryl may be substituted with one or more, typically 1-3, and often 1 or 2 substituents. Exemplary substituents of aryl group include, but are not limited to, those substituents described for heteroaryl.

“Heteroaryl” means a monovalent monocyclic or bicyclic aromatic moiety of 5 to 12 ring atoms containing one, two, or three ring heteroatoms selected from N, O, or S, the remaining ring atoms being C. The heteroaryl ring can be substituted with one or more substituents, typically one or more, often one to four, and more often one or two substituents. Suitable substituents include alkyl, haloalkyl, heteroalkyl, heterocyclyl, halo, nitro, cyano, carboxy, acyl, -(alkylene)ₙ-COOR (where n is 0 or 1 and R is hydrogen, alkyl, optionally substituted phenylalkyl, or optionally substituted heteroaralkyl), or -(alkylene)ₙ-CONR^{a}R^{b} (where n is 0 or 1, and R^{a} and R^{b} are, independently of each other, hydrogen, alkyl, cycloalkyl, cycloalkylalkyl, hydroxyalkyl, aryl, or R^{a} and R^{b} together with the nitrogen atom to which they are attached form a heterocyclyl ring). More specifically the term heteroaryl includes, but is not limited to, pyridyl, furanyl, thiophenyl, thiazolyl, isothiazolyl, triazolyl, imidazolyl, isoxazolyl, pyrrolyl, pyrazolyl, pyrazinyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like.

“Heterocycloalkyl” refers to a non-aromatic mono- or bicyclic moiety of three to twelve ring atoms in which one or more, typically one or two ring atoms are heteroatoms selected from N, O, or S(O)ₙ (where n is an integer from 0 to 2), the remaining ring atoms being C, where one or two C atoms can optionally be a carbonyl group. The heterocycloalkyl ring can be optionally substituted independently with one or more, typically one, two, or three, substituents. When two or more substituents are present in a heterocycloalkyl, each substituent is independently selected. Exemplary substituents for heterocycloalkyl include, but are not limited to, alkyl, haloalkyl, heteroalkyl, halo, nitro, cyano, optionally substituted phenyl, optionally substituted heteroaryl, optionally substituted phenyalkyl, optionally substituted heteroaralkyl, acyl,-(alkylene)ₙ-COOR (n is 0 or 1 and R is hydrogen, alkyl, optionally substituted phenyl, optionally substituted phenyalkyl, or optionally substituted heteroaralkyl), or -(alkylene)ₙCONR^{a}R^{b} (where n is 0 or 1, and R^{a} and R^{b} are, independently of each other, hydrogen, alkyl, cycloalkyl, cycloalkylalkyl, hydroxyalkyl, aryl, or R and R′ together with the nitrogen atom to which they are attached form a heterocyclyl ring). More specifically the term heterocyclo includes, but is not limited to, tetrahydropyranyl, piperidino, piperazino, morpholino, thiomorpholino, thiomorpholino-1-oxide, thiomorpholino-1,1-dioxide, and the like.

“(Heterocycloalkyl)alkyl” refers to a moiety of the formula —R^{a}R^{b}, where R^{b} is heterocycloalkyl and R^{a} is alkylene as defined herein.

“Alkynyl” means a linear monovalent hydrocarbon moiety of two to ten carbon atoms or a branched monovalent hydrocarbon moiety of three to ten carbon atoms, containing at least one carbon-carbon triple bond, e.g., ethenyl, propenyl, and the like.

“Heteroalkyl” means a branched or unbranched, cyclic or acyclic saturated alkyl moiety containing carbon, hydrogen and one or more heteroatoms in place of a carbon atom, or optionally one or more heteroatom-containing substituents independently selected from ═0, —OR^{a}, —C(O)R^{a}, —NR^{b}R^{c}, —C(O)NR^{b}R^{c} and —S(O)ₙR^{d} (where n is an integer from 0 to 2). R^{a} is hydrogen, alkyl, haloalkyl, cycloalkyl, cycloalkylalkyl, heterocyclyl, heterocyclylalkyl, aryl, aralkyl, heteroaryl, heteroaralkyl, or acyl. R^{b} is hydrogen, alkyl, haloalkyl, cycloalkyl, cycloalkylalkyl, heterocyclyl, heterocyclylalkyl, aryl, aralkyl, heteroaryl, heteroaralkyl, or acyl. R^{o} is hydrogen, alkyl, haloalkyl, cycloalkyl, cycloalkylalkyl, heterocyclyl, heterocyclylalkyl, aryl, aralkyl, acyl, alkyl sulfonyl, carboxamido, or mono- or di-alkylcarbomoyl. Optionally, R^{b} and R^{c} can be combined together with the nitrogen to which each is attached to form a four-, five-, six- or seven-membered heterocyclic ring (e.g., a pyrrolidinyl, piperidinyl or morpholinyl ring). R^{d} is hydrogen (provided that n is 0), alkyl, haloalkyl, cycloalkyl, cycloalkylalkyl, heterocyclyl, heterocyclylalkyl, aryl, aralkyl, heteroaryl, heteroaralkyl, acyl, amino, monsubstituted amino, disubstituted amino, or hydroxyalkyl. Representative examples of heteroalkyls include, but are not limited to, 2-methoxyethyl, benzyloxymethyl, thiophen-2-ylthiomethyl, 2-hydroxyethyl, 2,3-dihydroxypropyl, and guanidine derivative of the formula —C(═NR^{a})—NR^{b}R^{c} where each of R^{a}, R^{b} and R^{c} is independently H, alkyl, cycloalkyl, heterocycloalkyl, (heterocycloalkyl) alkyl, (cycloalkyl) alkyl, and heteroalkyl.

“Acyl” refers to a moiety of the formula —C(O)R′, where R′ is alkyl, haloalkyl, aryl, or aralkyl. “Sulfonyl” refers to a moiety of the formula —S(O)₂R^{y}, where R^{y} is alkyl, haloalkyl, optionally substitute aryl, optionally substituted aralkyl, or (cycloalkyl) alkyl. “Leaving group” has the meaning conventionally associated with it in synthetic organic chemistry, i.e., an atom or a group capable of being displaced by a nucleophile and includes halo (such as chloro, bromo, and iodo), alkanesulfonyloxy, arenesulfonyloxy, alkylcarbonyloxy (e.g., acetoxy), arylcarbonyloxy, mesyloxy, tosyloxy, trifluoromethanesulfonyloxy, aryloxy (e.g., 2,4-dinitrophenoxy), methoxy, N,O-dimethylhydroxylamino, and the like.

“Amino” or “amine” refers to a —N(R^{a})₂ radical group, where each R^{a} is independently hydrogen, alkyl, fluoroalkyl, carbocyclyl, carbocyclylalkyl, aryl, aralkyl, heterocycloalkyl, heterocyclylalkyl, heteroaryl or heteroarylalkyl, unless stated otherwise specifically in the specification. When a —N(R^{a})₂ group has two R^{a} other than hydrogen they can be combined with the nitrogen atom to form a 4-, 5-, 6-, or 7-membered ring.

The term “aromatic” means an aryl ring, wherein each atom that forms a ring is a carbon atom. An aryl ring is formed by 5, 6, 7, 8, 9, or more than 9 carbon atoms. The aryl group is substituted or unsubstituted. In one aspect, the aryl is phenyl or naphthalenyl. Depending on the structure, the aryl group may be a mono radical or a di-radical (i.e., an arylene group). In one aspect, the aryl is C₆-C₁₀ aryl.

“Alkoxy” refers to the group-O-alkyl, including from 1 to 8 carbon atoms of a straight, branched, cyclic configuration and combinations thereof attached to the parent structure through an oxygen. Examples include methoxy, ethoxy, propoxy, isopropoxy, cyclopropyloxy, cyclohexyloxy and the like. “Lower alkoxy” refers to alkoxy groups containing one to six carbons. In some embodiments, C₁-C₄ alkyl is an alkyl group which encompasses both straight and branched chain alkyls of from 1 to 4 carbon atoms.

“Substituted alkoxy” refers to alkoxy wherein the alkyl constituent is substituted (i.e., —O-(substituted alkyl)).

“Heteroaryl” or alternatively, “heteroaromatic” means a 5- to 18-membered aromatic radical (e.g., C₅-C₁₃ heteroaryl), which includes one or more ring heteroatoms selected from nitrogen, Atoms, and may be monocyclic, bicyclic, tricyclic or tetracyclic ring systems. As used herein, numerical ranges such as “5 to 18” mean each integer in a given range; For example, “5 to 18 ring atoms” means that the heteroaryl group can be composed of up to 18 ring atoms, such as 5 ring atoms, 6 ring atoms. The N-containing “heteroaromatic” or “heteroaryl” moiety refers to an aromatic group, wherein at least one of the backbone atoms of the ring is a nitrogen atom. The polycyclic heteroaryl groups may be fused or non-fused. The heteroatom(s) in the heteroaryl radical is optionally oxidized. The at least one nitrogen atom, if present, is optionally quaternized. Heteroaryl is attached to the remainder of the molecule through any ring(s) atom. Examples of heteroaryl include, but are not limited to, azepinyl, acridinyl, benzimidazolyl, benzindolyl, 1,3-benzodioxolyl, benzofuranyl, benzoxazolyl, benzo[d Benzothiadiazolyl, benzo [b] [1,4] dioxepinyl, benzo [b] [1,4] oxazinyl, 1,4-benzodioxanyl, benzonaphthofuranyl, benz Benzoxazinyl, benzothiazolyl, benzothienyl (benzothiophenyl), benzothiazolyl, benzothioxolyl, benzoxazolyl, benzoxazolyl, Benzothieno [3,2-d] pyrimidinyl, benzotriazolyl, benzo [4,6] imidazo [1,2-a] pyridinyl, carbazolyl, cinnolinyl, cyclopenta [d] carbonyl, 6,7-dihydro-5 h-cyclopenta [4,5] thieno [2,3-d] pyrimidinyl, 5,6-dihydrobenzo [h] quinazolinyl, 5,6-dihydrobenzo [h] Shin fun carbonyl, 6,7-dihydro-5H-benzo [6,7] cyclo hepta [1,2-c] pyridazinyl, Benzofuranyl, dibenzo-thiophenyl, furanyl, furanyl janil, nonyl furanyl, furo [3,2-c] pyridinyl, 5,6,7,8,9,10-hexahydro-cyclooctadiene [d] pyrimidinyl Yl, 5,6,7,8,9,10-hexahydrocycloocta [d] pyridazinyl, 5,6,7,8,9,10-hexahydrocycloocta [d] pyridinyl, isothiazolyl, Imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a, 7,8,9, 10,10a-octahydro-benzo [h] quinazolinyl, 1-phenyl-1H-pyrrolyl, penah possess, phenothiazine thiazinyl, oxazolyl pen possess, phthalazine possess, loop terry pyridinyl, Fourier carbonyl, pyranyl, Pyrrolyl, pyrazolyl, pyrazolol [3,4-d] pyrimidinyl, pyridinyl, pyrido [3,2-d] pyrimidinyl, pyrido [3,4-d] pyrimidinyl, Pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydro-benzo [4,5] thieno [2,3-d] pyrimidinyl, 6,7,8,9-tetrahydro-5H-cyclo hepta [4,5] Thieno [2,3-d] pyrimidinyl, 5,6,7,8-tetrahydropyrido [4,5-c] pyridazinyl, thiazolyl, thiadiazolyl, thiapyranyl, triazolyl, tetrazolyl, triazinyl, thieno [2,3-d] pyrimidinyl, thieno [3,2-d] pyrimidinyl, thieno [2,3-c] pyridinyl, and thiophenyl (i.e., Thienyl). Unless specifically stated otherwise herein, the heteroaryl moiety is optionally substituted by one or more substituents independently selected from the group consisting of alkyl, heteroalkyl, alkenyl, alkynyl, cycloalkyl, heterocycloalkyl, aryl, arylalkyl, heteroaryl, heteroarylalkyl, hydroxy, halo, cyano, nitro, oxo, thioxo, trimethylsilanyl, —OR a, —SR a, —OC (O) R a, —N (R a) 2, —C (O) R a, —C (O) OR a, —C (O) N (R a) 2, —N (R a) C (O) OR a, —N (R a) (O) t R a, —N (R a) S (O) t R a wherein tis 1 or 2, —S (O) t OR a wherein t is 1 or 2, Or —S (O) tN (R a) 2 wherein t is 1 or 2, and each R a is independently hydrogen, alkyl, fluoroalkyl, carbocyclyl, carbocyclylalkyl, Aryl, aralkyl, heterocyclyl, heterocyclylalkyl, heteroaryl or heteroarylalkyl.

The term “substituted or unsubstituted aromatic hydrocarbon group” means a monocyclic or polycyclic aromatic hydrocarbon group which may have one or more of various substituents on the ring. Examples are: phenyl, methylphenyl, dimethylphenyl, methoxyphenyl, dimethoxyphenyl, fluorophenyl, dinitrophenyl, trifluoromethylphenyl, dimethylaminophenyl, mercaptophenyl, α-naphthyl, and β-naphthyl groups.

The term “substituted or unsubstituted aromatic heterocyclic group” means a group in the form of a tour-membered, five-membered, six-membered, seven-membered, eight-membered, or nine-membered ring comprising one or more hetero atoms such as nitrogen atoms, sulfur atoms, oxygen atoms, phosphorus atoms. These may be condensed into a benzene ring. There may be one or more of a variety of substituents on the ring. Examples are: pyridyl, furyl, thienyl, indolyl, quinolyl, isoquinolyl, benzofuranyl, benzothienyl, imidazolyl, benzimidazolyl, thiazolyl, oxazolyl, pyrazolyl, pyrimidyl, pyradinyl, pyridazyl, isooxazolyl, isoindolyl, and pyrrolyl.

The term “substituted or unsubstituted cycloalkane” means a cyclic alkane having 3 to 20 carbon atoms, preferably 3 to 12 carbon atoms, that is monocyclic or polycyclic and may have one or more of various substituents on the ring.

The terms “alkenyl group, alkynyl group, alkoxy group, and alkylthio group with 2 to 12 carbon atoms, 1 to 6 carbon atoms” means straight chain, cyclic, or branched chain, examples of which are the same as those given for the alkyls. These alkenyl groups, alkynyl groups, alkoxy groups, and alkylthio groups may be further substituted with various substituents.

In all of the embodiments of this invention (both above and below herein), the substituents (each of the R groups) are not especially limited, provided that they do not prevent the RMD function from occurring. In all of the embodiments mentioned in connection with this invention, both above and in the following, the substituents are selected from H and an organic group. Thus, both above and in the following, the terms ‘substituent’ and ‘organic group’ are not especially limited and may be any functional group or any atom, especially any functional group or atom common in organic chemistry. Thus, ‘substituent’ and ‘organic group’ may have any of the following meanings.

The organic group may comprise any one or more atoms from any of groups IIIA, IVA, VA, VIA or VIIA of the Periodic Table, such as a B, Si, N, P, O, or S atom (e.g. OH, OR, NH₂, NHR, NR₂, SH, SR, SO₂R, SO₃H, PO₄H₂) or a halogen atom (e.g. F, Cl, Br or I) where R is a linear or branched lower hydrocarbon (1-6 C atoms) or a linear or branched higher hydrocarbon (7 C atoms or more, e.g. 7-40 C atoms). The organic group preferably comprises a hydrocarbon group. The hydrocarbon group may comprise a straight chain, a branched chain or a cyclic group. Independently, the hydrocarbon group may comprise an aliphatic or an aromatic group. Also independently, the hydrocarbon group may comprise a saturated or unsaturated group. When the hydrocarbon comprises an unsaturated group, it may comprise one or more alkene functionalities and/or one or more alkyne functionalities. When the hydrocarbon comprises a straight or branched chain group, it may comprise one or more primary, secondary and/or tertiary alkyl groups. When the hydrocarbon comprises a cyclic group it may comprise an aromatic ring, an aliphatic ring, a heterocyclic group, and/or fused ring derivatives of these groups. The cyclic group may thus comprise a benzene, naphthalene, anthracene, indene, fluorene, pyridine, quinoline, pyrrolidine, piperidine, morpholine, thiophene, benzothiophene, furan, benzofuran, pyrrole, indole, imidazole, thiazole, diazole, and/or an oxazole group, as well as regioisomers of the above groups. The number of carbon atoms in the hydrocarbon group is not especially limited, but preferably the hydrocarbon group comprises from 1-40 C atoms. The hydrocarbon group may thus be a lower hydrocarbon (1-6 C atoms) or a higher hydrocarbon (7 C atoms or more, e.g. 7-40 C atoms). The lower hydrocarbon group may be a methyl, ethyl, propyl, butyl, pentyl or hexyl group or regioisomers of these, such as isopropyl, isobutyl, tert-butyl, etc. The number of atoms in the ring of the cyclic group is not especially limited, but preferably the ring of the cyclic group comprises from 3-10 atoms, such as 3, 4, 5, 6, 7, 8, 9 or 10 atoms.

The groups comprising heteroatoms described above, as well as any of the other groups defined above, may comprise one or more heteroatoms from any of groups IIIA, IVA, VA, VIA or VIIA of the Periodic Table, such as a B, Si, N, P, O, or S atom or a halogen atom (e.g. F, Cl, Br or I). Thus the substituent may comprise one or more of any of the common functional groups in organic chemistry, such as hydroxy groups, carboxylic acid groups, ester groups, ether groups, aldehyde groups, ketone groups, amine groups, amide groups, imine groups, thiol groups, thioether groups, sulphate groups, sulphonic acid groups, sulphonyl groups, and phosphate groups etc. The substituent may also comprise derivatives of these groups, such as carboxylic acid anhydrides and carboxylic acid halides.

In addition, any substituent may comprise a combination of two or more of the substituents and/or functional groups defined above.

“Pharmaceutically acceptable excipient” refers to an excipient that is useful in preparing a pharmaceutical composition that is generally safe, non-toxic and neither biologically nor otherwise undesirable, and includes excipient that is acceptable for veterinary use as well as human pharmaceutical use.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated. “Treating” or “treatment” of a disease includes: (1) preventing the disease, i.e., causing the clinical symptoms of a bacterial infection not to develop in a mammal that may be exposed to or predisposed to the infection but does not yet experience or display symptoms of the infection; (2) inhibiting the disease, i.e., arresting or reducing the development of the infection or its clinical symptoms; or (3) relieving the disease, i.e., causing regression of the infection or its clinical symptoms.

When describing a chemical reaction, the terms “treating”, “contacting” and “reacting” are used interchangeably herein and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product. As used herein, the terms “those defined above” and “those defined herein” when referring to a variable incorporates by reference the broad definition of the variable as well as any narrow definitions, if any.

As used herein, the following abbreviations are defined as: MRSA, methicillin-resistant S. aureus; RMA, resistance-modifying agent; MSSA, methicillinsensitive S. aureus; CLSI, Clinical Laboratory Standards Institutes; SAR, structure-activity relationship; MIC, minimal inhibitory concentration; MRC, minimum resensitizing concentration; GI₅₀, half growth inhibitory concentration; HeLa cells, human cervical carcinoma cells; HAIs, hospital acquired infections; CLSI, Clinical & Laboratory Standards Institute; DMSO, dimethyl sulfoxide; TFA, trifluoroacetic acid; Rt, LCMS retention time; DIEA, N,N-Diisopropylethylamine.

The invention now being generally described will be more readily understood by reference to the following examples, which are included merely for the purposes of illustration of certain embodiments of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Colistin RMA Screen

A MDR E. coli strain AR-0493 (a.k.a., ATCC BAA-3170) was obtained from the CDC and FDA Antibiotic Resistance Bank and used for the initial screen. The present inventors first determined the minimum inhibitory concentration (MIC) of colistin as 16 μg/mL in this strain using the standard Clinical Laboratory Standards Institute (CLSI) broth microdilution method. Considering the difficulties on the discovery of novel antimicrobial reagents against Gram-negative bacteria, we chose to screen the fragment-based library of 3,200 compounds from TimTec (Tampa, FL). These compounds have low molecular weights ranging from 110 to 290 and relatively high aqueous solubility, making them an ideal library to screen against Gram-negative bacteria. The screen used a modified broth microdilution assay.

Compounds were screened at the final concentration of 75 μM (c.a., 8-20 μg/mL) in 96-well plates in duplicate, and the cation-adjusted MHB (CAMHB) medium was supplemented with 2 μg/mL of colistin, its clinical breakpoint for intermediate resistant Enterobacteriaceae. After incubation at 35° C. over 19 hours, 34 compounds, in combination with colistin, inhibited bacterial growth. These compounds were selected for confirmation using the same assay, where each compound was tested in 4 concentrations (i.e., 75, 37.5, 18.7, 9.4 μM) in duplicate. The 10 most active compounds were identified as hits and re-purchased from TimTec for further validation.

### Example 2: Identification of MICs and Minimum Re-Sensitizing Concentrations (MRCs) in the Presence Colistin

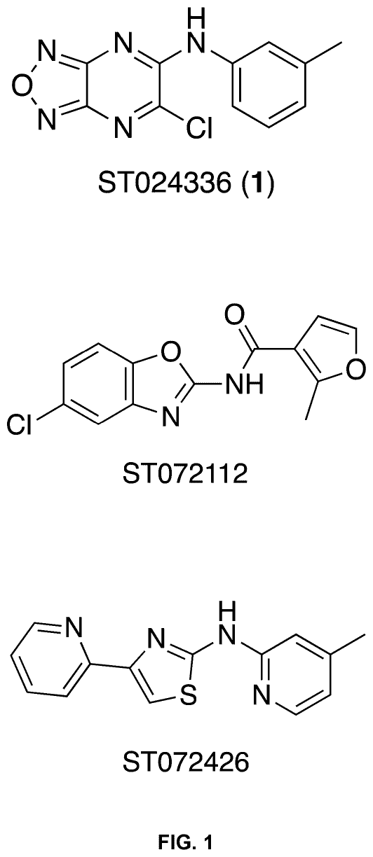

These 10 compounds were tested for their MICs and minimum re-sensitizing concentrations (MRCs) in the presence colistin at 2 μg/mL in E. coli AR-0493. Three out of (see structures in FIG. 1) showed the desired activity and selectivity with MICs >32 μg/mL and MRCs≤4 μg/mL. To evaluate their toxicity in mammalian cells, the present inventors treated human epithelial adenocarcinoma HeLa cells with each compound at a wide range of concentrations. After incubation at 37° C. over 24 hours, the remaining cells were analyzed using CellTiter-Glo® mammalian viability assay (Promega) and their half growth inhibition concentrations (GI50s) were determined by fitting the data using KaleidaGraph (Synergy Software). The GI50 of ST024336 (1, FIG. 1) was determined as 10 μg/mL, 5 times of its MRC (i.e., 2 μg/mL), while the GI50s of the other two were 0.9 and 2.0 μg/mL, respectively, lower than their respective MRCs (i.e., 2 and 4 μg/mL). Based on these data, ST024336 (1) was selected for further study as described below.

### Example 3: Synthesis of RMC Compounds

Next, compound 1 re-synthesized by following Santos' procedure. Compound 1 was obtained by treating meta-toluidine with excess dichloride (2, Scheme 1A) in the presence of triethylamine. Notably, the purified compound 1 was unable to potentiate colistin in AR-0493 at up to 64 μg/mL, the highest concentration tested. This suggested that some impurity in the commercial sample may be responsible for the desired RMA activity. During the synthesis of 1, the present inventors noticed that a small amount of dimer 3 (Scheme 1A) was also formed, which was difficult to separate from 1 due to their similar polarity. In addition, compound 1 is also readily hydrolyzed to the hydroxide analog 4 under basic aqueous condition. Mass spectrometry analysis of the commercial compound confirmed this hypothesis and detected the mass of both compounds. Therefore, we synthesized and purified these two compounds using their respectively optimized procedures.

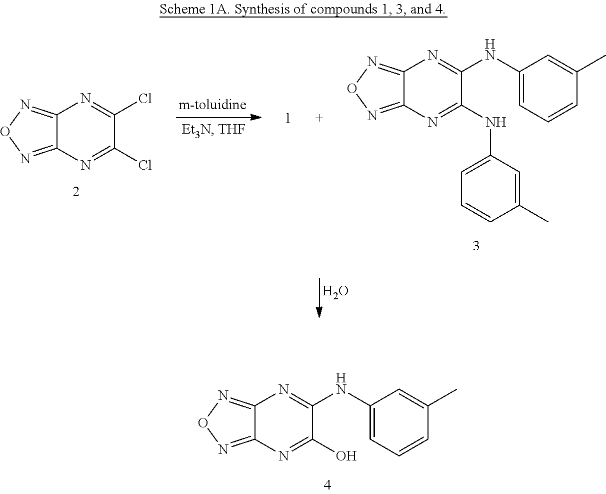 [SMILES: Clc1nc2nonc2nc1Cl ; Cc1cccc(Nc2nc3nonc3nc2Nc2cccc(C)c2)c1 ; O ; Cc1cccc(Nc2nc3nonc3nc2O)c1]

### Example 4: Identification of Synergistic Effect of Compound 3 and Colistin

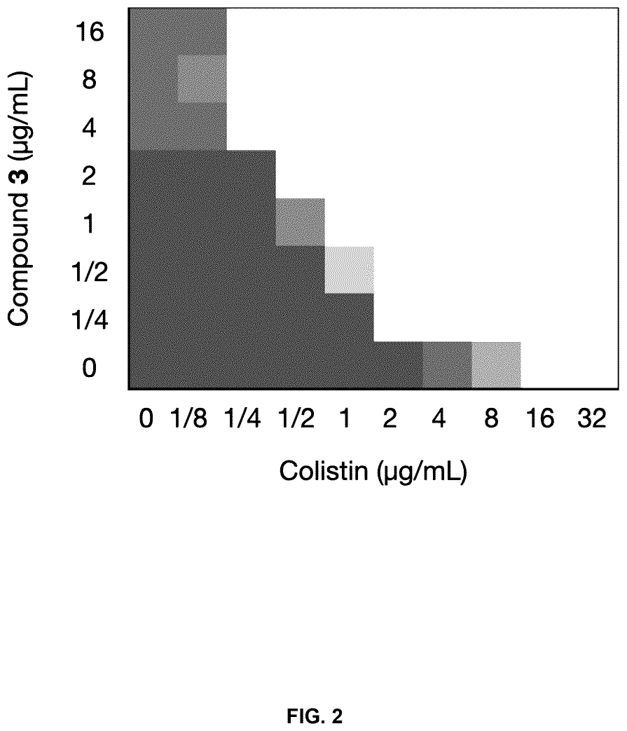

Compound 3 (represented as the compound of Formula VI) is highly active with an MRC of 0.5 g/mL in AR-0493 and showed no antimicrobial activity at up to 64 μg/mL. Compound 4, however, did not show either antimicrobial or RMA activity. To further confirm the synergistic effect of compound 3 and colistin, the checkerboard broth microdilution assay was performed (FIG. 2). The fractional inhibitory concentration index (FICI) of compound 3 and colistin was calculated as <0.06, which is significantly lower than 0.5 and suggests a strong synergistic effect. The GI₅₀ of compound 3 in HeLa cells was also determined as 15 μg/mL, which is 30 times higher than its MRC.

The present inventors next screened a panel of 9 commonly used antibiotics for Gram-negative bacteria (Table 1) for changes in MICs in the presence of 1 μg/mL of compound 3 in E. coli AR-0349. At this concentration, compound 3 was able to enhance the activity of both colistin and polymyxin B by 16 folds and lower their MICs from 16 to 1 μg/mL. In addition to polymyxins, this strain is also resistant to ceftazidime (a third-generation cephalosporin), azithromycin, ciprofloxacin, rifampicin, and tetracycline. Interestingly, compound 3 did not affect the MICs of any of these antibiotics. In addition, compound 3 did not affect the MICs of two other antibiotics, gentamycin and meropenem, that this MDR strain is susceptible to. These results suggested that compound 3 is a highly selective RMA for polymyxin family antibiotics, but not any other classes of antibiotics.

### Example 5: Identification of Colistin-Potentiation Effect of Compound 3

To investigate the scope of the colistin-potentiation effect of compound 3, the present inventors screened a panel of MDR Gram-negative bacteria obtained from the CDC and FDA Antibiotic Resistance Bank. These include a variety of Enterobacteriaceae spp., such as E. coli AR-0346 and AR-0538, K. pneumoniae AR-0497, Salmonella Enteritidis AR-0496, and Salmonella Typhimurium AR-0635. These strains all express the mcr genes (e.g., mcr-1, mcr-2, and mcr-4) and are resistant to colistin with its MICs of 8-16 μg/mL (Table 2). When used at 1 μg/mL, compound 3 showed 16 folds of potentiation for colistin and lowered its MICs to 0.5-1 μg/mL for almost all these strains. The only exception is Salmonella Typhimurium AR-0635, which expresses mcr-4 gene. In this strain, 1 μg/mL of compound 3 was only able to lower the MIC of colistin to 4 μg/mL, and 2 μg/mL of 3 was required to lower the MIC of colistin to 1 μg/mL. We next tested another two A. baumannii strains from the same sources. Compound 3 showed even more potent effect in this species with 32 folds of potentiation for colistin. Therefore, at 1-2 μg/mL, compound 3 re-sensitize all these MDR Gram-negative bacteria to colistin and lower its MICs below 2 μg/mL, the clinical breakpoint. Furthermore, the present inventors also tested the activity of colistin in the presence or absence of 1 μg/mL compound 3 in a wild-type E. coli K-12. The MICs of colistin were found to be 0.25 μg/mL, regardless of the presence of compound 3. The MICs of compound 3 in all these strains were >64 μg/mL, the highest concentration tested. Taken together, these results suggested that compound 3 is a highly active and selective RMA for polymyxin family antibiotics in resistant Gram-negative bacteria.

The present inventors further conducted MRC screens of selected compounds from groups 2-5 in AR-0493. Compound were tested against AR-0493 with compound X+2 μg/mL Colistin. Results are provided in Table 3 below

### Example 5. Synthesis Schemes

The present inventors synthesized a plurality of derivative compounds using the procedures A, B or C as identified below.
Procedure A: 5,6-Dichloro-[1,2,5]oxadiazolo[3,4-b]pyrazine (1, 1.0 equiv) was dissolved in THF (0.60 M solution). The desired substituted arylamine (4.0 equiv) was then added and the reaction mixture was heated to reflux overnight. After which, the resulting mixture was concentrated under reduced pressure, and the residue was purified by column chromatography on silica gel to yield the title compound. For 2n-2p, the 3-trifluoromethoxy aniline (0.90 equiv) was added at 0° C. to dichloride for monochloride intermediate, then desired substituted aryl amines were added to it and stirred at room temperature for hours. The mixture was concentrated under reduced pressure and purified by column chromatography.

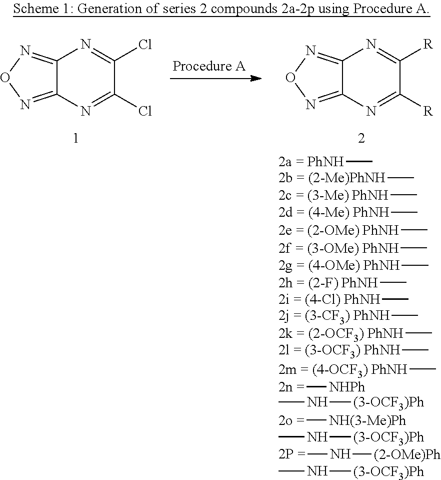

Procedure B: In a screw-cap vial or round-bottom flask, the requisite aniline (0.90 equiv) was added to a stirring mixture of dichloro 1 (1.0 equiv) in anhydrous THF (0.1-0.2 M). Then, Et₃N (1.1 equiv) was added and the resulting dark mixture was stirred at room temperature (unless otherwise indicated) for 2-20 h. The reaction mixture was diluted with an aqueous solution of KOH (6.0 equiv, 1.0 M), and stirring at room temperature was continued for 0.5-2 h. The mixture was acidified with 1.0 M aqueous HCl and extracted with EtOAc. The organic layer was washed with brine, dried over Na₂SO₄, filtered, and concentrated to a residue. The residue was purified by chromatography on SiO₂ using a MeOH/CH₂Cl₂ or EtOAc/hexanes solvent system to yield the desired product.

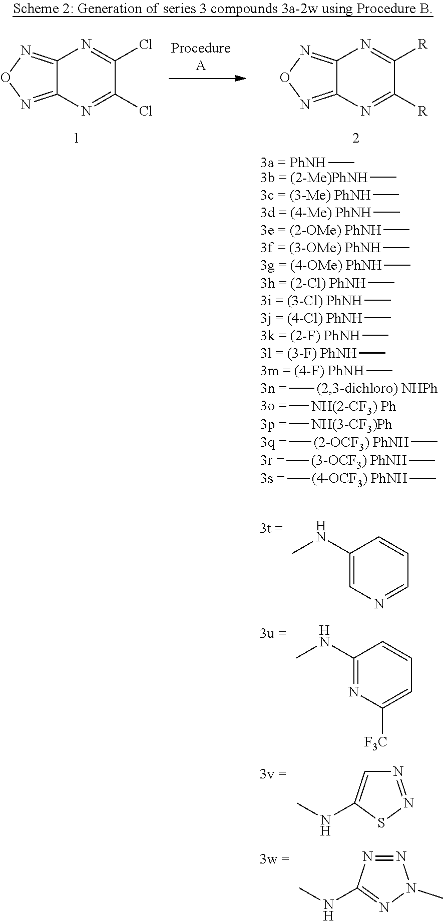

Procedure C: 5,6-Dichloro-[1,2,5]oxadiazolo[3,4-b]-pyrazine (1, 1.0 equiv) was dissolved in THF (0.52 M solution) and cooled to 0° C. Then, 4-trifluoromethoxy aniline (for 4) or 3-trifluoromethoxy aniline (for 5) (0.90 equiv) was added dropwise and allowed to stir for 10 min. Triethylamine (1.0 equiv) was then added dropwise and allowed to stir at RT for an additional 1 h. After that the amino alcohols or hydroxyl amine were added (4 equiv) and allowed to stir at RT for 2 hours. The mixture was acidified with 1.0 M aqueous HCl and extracted with EtOAc. The organic layer was washed with brine, dried over Na₂SO₄, filtered, and concentrated to a residue. The residue was purified by chromatography on SiO₂ using a MeOH/CH₂Cl₂ or EtOAc/hexanes solvent system to yield the desired product.

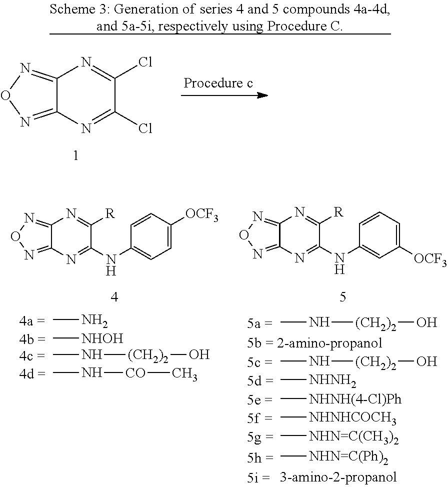

### Example: 8: Synthesis of Analogs Bearing Different Substituents of the Anilines

A variety of analogs of 2d and 3d bearing different substituents of the anilines, 7a-s and 8a-p (Scheme 5), were synthesized by the Applicant's. The synthesis began with the reaction of commercially available diaminofurazan (3x), oxalic acid, and aqueous 10% HCl solution, followed by chlorination with PCl₅/POCl₃ to afford dichloride 4x (Scheme 5). Dichloride 4x was then treated with various anilines in the presence of triethylamine in THF and refluxed overnight to provide bisanilino derivatives 7a-p. To synthesize analogs 8a-p, 4 was reacted with the aniline at room temperature followed by hydroxylation with aqueous potassium hydroxide and then quenching with dilute hydrochloric acid. For asymmetrical bisanilino derivatives, intermediate 4x was treated with 1.0 equivalent of meta-trifluoromethoxy aniline and triethylamine in THF at 0-25° C. for 1 h followed by the addition of the second anilines. The resulting mixture was then refluxed overnight to produce 7q-s.

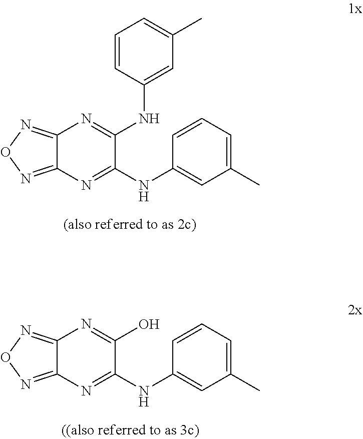 [SMILES: Cc1cccc(Nc2nc3nonc3nc2Nc2cccc(C)c2)c1 ; Cc1cccc(Nc2nc3nonc3nc2O)c1]

### Structures of Two Exemplary RMAs for Colistin-Resistant Gram-Negative Bacteria.

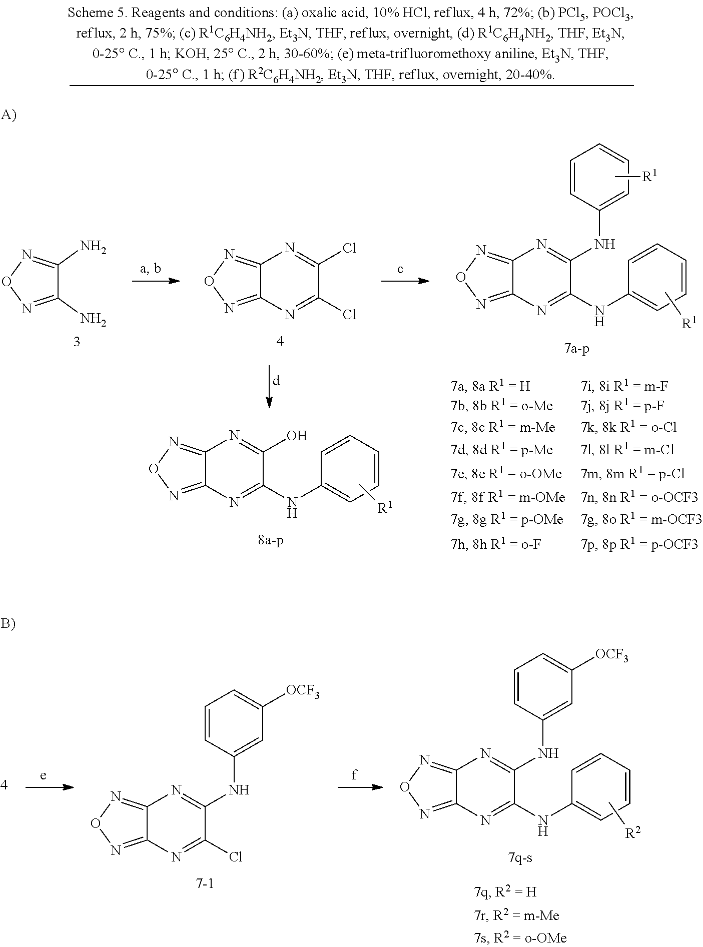

All compounds were tested for their respective minimum resensitizing concentrations (MRCs) for colistin against E. coli. AR0493, a MDR strain from CDC and FDA Antibiotic Resistance Bank. Compounds with good RMA activity with MRCs <1 μg/mL were also tested for their minimum inhibitory concentrations (MICs). For the symmetrical bisanilino series, 7a-p, electron-donating alkyl and alkoxy groups (entries 7a-g, Table 5) at various positions of the phenyl rings were investigated. Only 7d with para-Me and 7f with meta-OMe substitutions showed similar RMA activity as 1x with meta-Me substitutions. Substitutions at the ortho positions significantly reduced the RMA activity. Substitutions on the phenyl groups using electronwithdrawing groups (e.g., F, Cl, and OCF3) appeared to benefit the RMA activity in many cases. Among these three groups, trifluoromethoxy groups with the strongest electron-withdrawing capability and highest hydrophobicity showed the best RMA activity overall, and the analogs with ortho substitutions were still the worst within each series. Three analogs with meta-Cl, meta-OCF3, and para-OCF3 (i.e., 7l, 57o, 57p), respectively, showed the best RMA activity with MRCs of 0.25 μg/mL and no antibacterial activity on their own with MICs>64 μg/mL, the highest concentration tested. Three asymmetric bisanilino analogs (7q-s, Table 5) were synthesized based on the SARs of the monoanilino analogs 8a-p (vide infra). However, none of them showed improved RMA activity.

To evaluate the mammalian toxicity of these analogs, Applicants tested them in cell viability assay using human cervical adenocarcinoma HeLa cells. Their respective half maximal growth inhibition concentration (GI₅₀) was calculated using KaleidaGraph. The selectivity index (SI) for each compound was also calculated as the ratio of their GI50 and MRC. Compared with the lead compound, 1x (7c), three analogs (i.e., 7j, 7l and 7m), showed improved SIs. Although two analogs bearing trifluoromethoxy substitutions (i.e., 7n and 7o) are more potent RMAs, they were also more toxic than compound 1x, resulting in reduced SIs. Therefore, Applicants selected two bisanilino analogs, 7l and 7m, that bear chlorines at the meta- and para-positions, respectively, of the phenyl groups for further characterization studies. For the monoanilino analogs, 8a-p, their RMA activities appeared to be considerably (>10 folds) lower than their corresponding bisanilino analogs. Only two compounds, 8e (ortho-OMe, Table 6) and 8o (meta-OCF₃, Table 6) showed slightly improved activity with MRCs of 4 μg/mL, when compared to the parent compound 2x (vide supra). Their mammalian toxicities were also evaluated, and SIs calculated. The results showed that 8e bearing an ortho-OMe group has very weak toxicity in mammalian cells with a GI50>100 μg/mL, the highest concentration tested, while 8o has a moderate SI. Applicants we next explored the scope of analogs 71 and 7m their colistin-potentiating activity in 8 different MDR Gram negative bacteria with diverse genetic backgrounds and resistance mechanisms (Table 7). All strains were obtained from CDC and FDA Antibiotic Resistance Bank. For example, E. coli AR-0493 and K. pneumoniae AR-0497 carry the plasmid that contains the most prevalent mcr-1 gene. E. coli AR-0538, Salmonella Typhimurium AR-0539 and AR-0635 contain the mcr-2, 3, and 4 genes, respectively. These genes encode the phosphoethanolamine transferase, which was originated form the chromosome of A. baumannii, such as AR-0310. P. aeruginosa, on the other hand, contains genes that are responsible for the Ara4N modification of Lipid A. These strains are all resistant to colistin with their MICs in the range of 4-32 μg/mL. When used at 1 μg/mL, compounds 71 and 7m reduced the colistin MIC to 1-2 μg/mL in strains that contain the mcr genes. A. baumannii AR-0310 appears more sensitive to these two compounds and the MICs of colistin was reduced to 0.25 μg/mL. Interestingly, both compounds are also effective in two P. aeruginosa strains and reduced the MICs of colistin in both AR-0239 and AR-0257 by 4 folds. These results suggest that the oxadiazolopyrazine containing compounds do not inhibit the activity of pEtN transferase directly, but resensitize these resistant bacteria to colistin via a novel mechanism.

Both series of compounds had previously been reported as mitochondrial protonophores uncouplers. They are weak lipophilic acids that uncouple oxidative phosphorylation from ATP production by transporting protons across the inner membrane of mitochondria into the mitochondrial matrix. The bisanilino compounds (e.g., 1x) were more potent in the cellular oxygen consumption assay. However, they suffer from high hydrophobicity and poor aqueous solubility. A para-OCF3 analog of the monoanilino compound 2x possesses more favorable pharmacological properties, albeit with slightly lower activity in the cellular oxygen consumption assay. The SARs of these compounds as RMAs and those as mitochondria protonophore uncouplers are similar in certain aspects, but different in others. For example, analogs with higher pKa and hydrophobicity are preferred and the bisanilino analogs are much more potent than their corresponding monoanilino analogs. However, monoanilino analog 8p bearing a para-OCF₃ substitution was one of the most potent uncouplers both in vitro and in mice, yet with poor RMA activity. Bisanilino analog 7p bearing para-OCF3 substitutions was a poor uncoupler, but with very potent RMA activity. Taken together, these comparisons suggest that these compounds may potentiate colistin via a novel mechanism, other than shuffling protons across the membrane.

In summary, Applicants synthesized a variety of both bisanilino and monanilino [1,2,5]oxadiazolo[3,4-b]pyrazine-containing compounds and evaluated their antibacterial and colistin-resensitizing activity, as well as the mammalian toxicity. The SARs of both series of compounds were established and suggested that the bisanilino analogs are significantly more potent RMAs, and the analogs with electron-withdrawing and hydrophobic groups on the meta- or para-positions of the phenyl rings are beneficial to the RMA activity, while substitutions at the orth opositions have deleterious effect. Two bisanilino analogs bearing meta-Cl and para-Cl substitutions, respectively, were identified with improved activity or mammalian toxicity. Further evaluation of these in a wide range of MDR Gram-negative bacteria showed that they are effective RMAs in all species and strains tested and were able to lower the MICs of colistin to its clinical breakpoint, 2 μg/mL or lower. Since P. aeruginosa is resistant to colistin by Ara4N modification of Lipid A, the results suggested that these compounds do not inhibit the pEtN transferase directly and may potentiate colistin via a novel mechanism.

### Example: 9: Synthesis of Unsymmetrical Derivatives Via One-Pot Reaction

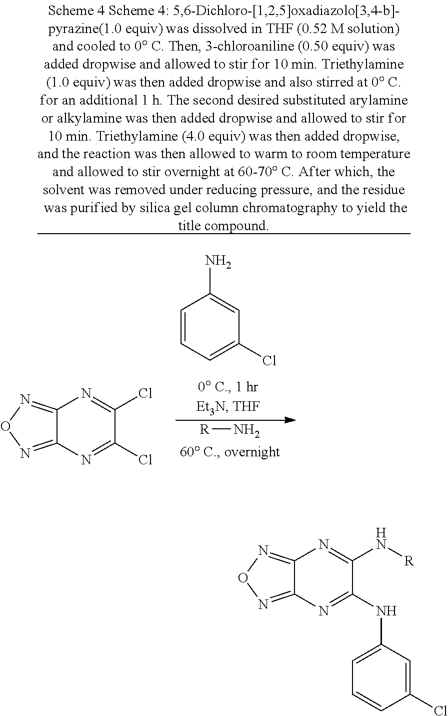

### Example: 10: Exemplary Characterization Data

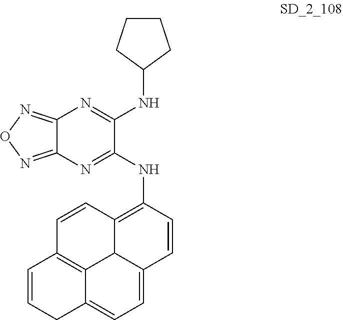 [SMILES: C1=CC2=C3C(=CC=C4C=CC(Nc5nc6nonc6nc5NC5CCCC5)=C(C=C2)C43)C1]

SD_2_108, yellow solid, 30% yield, ¹H NMR (CDCl₃, 300 MHz) δ 8.25-8.20 (m, 3H), 8.11-8.03 (m, 4H), 7.94 (d, J=9 Hz, 1H), 7.67-7.58 (m, 1H), 4.68-4.59 (m, 1H), 2.34-2.24 (m, 2H), 1.90-1.68 (m, 6H) ppm.

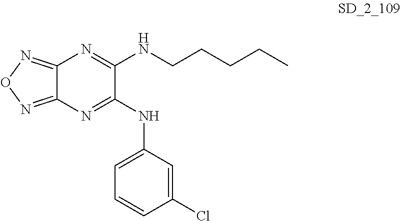 [SMILES: CCCCCNc1nc2nonc2nc1Nc1cccc(Cl)c1]

SD_2_109, yellow solid, 43% yield, ¹H NMR (CDCl₃, 300 MHz) δ 7.39 (t, J=8.0 Hz, 1H), 7.24-7.18 (m, 1H), 7.10-6.89 (m, 1H), 3.61 (t, J=7.3 Hz, 2H), 1.72-1.73 (m, 2H), 1.51-1.32 (m, 4H), 1.04-0.82 (m, 3H) ppm.

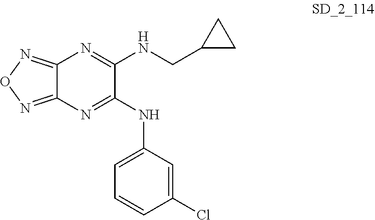 [SMILES: Clc1cccc(Nc2nc3nonc3nc2NCC2CC2)c1]

SD_2_114, yellow solid, 42% yield, 1H NMR (300 MHz, CDCl₃) δ 8.92 (s, 1H), 7.36 (td, J=8.0, 0.4 Hz, 1H), 7.17 (ddd, J=8.1, 2.1, 1.0 Hz, 1H), 6.98 (td, J=2.0, 0.4 Hz, 1H), 6.93-6.80 (m, 1H), 3.43 (ddd, J=7.0, 5.2, 1.9 Hz, 2H), 1.23-1.06 (m, 1H), 0.69-0.60 (m, 2H), 0.39-0.31 (m, 2H) ppm.

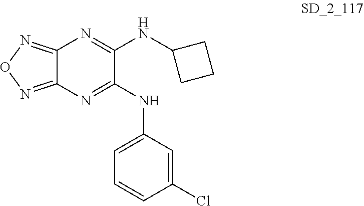 [SMILES: Clc1cccc(Nc2nc3nonc3nc2NC2CCC2)c1]

SD_2_117, yellow solid, 44% yield, ¹H NMR (300 MHz, CDCl₃) δ 7.87 (s, 1H), 7.62 (d, J=8.2 Hz, 1H), 7.44 (td, J=8.1, 0.4 Hz, 1H), 7.23 (ddd, J=8.0, 2.1, 1.0 Hz, 1H), 4.65-4.68 (m, 1H), 2.56-2.40 (m, 2H), 1.91-1.78 (m, 4H) ppm.

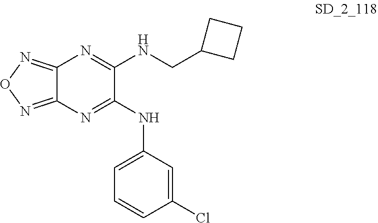 [SMILES: Clc1cccc(Nc2nc3nonc3nc2NCC2CCC2)c1]

SD_2_118, yellow solid, 45% yield, ¹H NMR (300 MHz, CDCl₃) δ 7.41-7.33 (m, 1H), 7.24-7.13 (m, 1H), 7.06-6.81 (m, 1H), 3.63 (dd, J=7.4, 5.8 Hz, 2H), 2.71-2.72 (m, 1H), 2.18-2.10 (m, 2H), 2.02-1.91 (m, 2H), 1.88-1.74 (m, 2H) ppm.

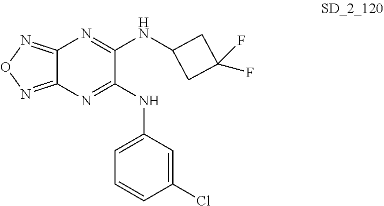 [SMILES: FC1(F)CC(Nc2nc3nonc3nc2Nc2cccc(Cl)c2)C1]

SD_2_120, yellow solid, 40% yield, ¹H NMR (300 MHz, CDCl₃) δ 8.60 (s, 1H), 7.41 (td, J=8.0, 0.4 Hz, 1H), 7.23 (ddd, J=8.1, 2.0, 1.0 Hz, 1H), 6.99 (td, J=2.0, 0.4 Hz, 1H), 6.87 (ddd, J=7.9, 2.0, 1.0 Hz, 1H), 4.66-4.45 (m, 1H), 3.33-3.11 (m, 2H), 2.84-2.56 (m, 2H) ppm.

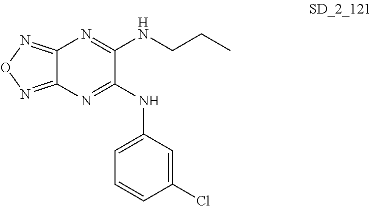 [SMILES: CCCNc1nc2nonc2nc1Nc1cccc(Cl)c1]

SD_2_121, yellow solid, 44% yield, ¹H NMR (300 MHz, CDCl₃) δ 7.36 (t, J=8.0 Hz, 1H), 7.17 (ddd, J=8.1, 2.1, 1.0 Hz, 1H), 6.97 (t, J=2.0 Hz, 1H), 6.85 (ddd, J=7.9, 2.0, 1.0 Hz, 1H), 3.56 (ddd, J=8.0, 7.2, 6.0 Hz, 2H), 1.74-1.76 (m, 2H), 1.05 (t, J=7.4 Hz, 3H) ppm.

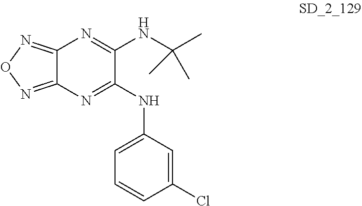 [SMILES: CC(C)(C)Nc1nc2nonc2nc1Nc1cccc(Cl)c1]

SD_2_129, yellow solid, 47% yield, ¹H NMR (300 MHZ, CDCl₃) δ 8.63 (s, 1H), 7.38 (ddd, J=14.4, 8.8, 4.8 Hz, 2H), 7.26-7.13 (m, 1H), 6.98 (s, 1H), 6.85 (d, J=7.9 Hz, 1H), 1.59 (s, 9H) ppm.

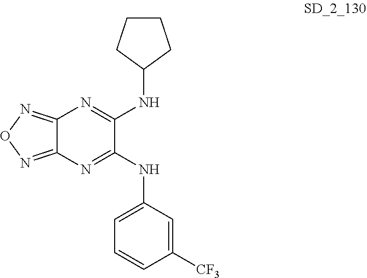 [SMILES: FC(F)(F)c1cccc(Nc2nc3nonc3nc2NC2CCCC2)c1]

SD_2_130, yellow solid, 46% yield, ¹H NMR (300 MHz, CDCl₃) δ 8.86 (s, 1H), 7.62-7.53 (m, 1H), 7.46 (ddt, J=7.8, 1.8, 1.0 Hz, 1H), 7.25-7.22 (m, 1H), 7.18-7.12 (m, 1H), 4.48-4.49 (m, 1H), 2.16 (dddd, J=12.6, 6.5, 3.4, 1.7 Hz, 2H), 1.79-1.55 (m, 6H) ppm.

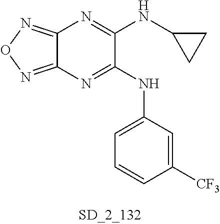 [SMILES: FC(F)(F)c1cccc(Nc2nc3nonc3nc2NC2CC2)c1]

SD_2_132, yellow solid, 47% yield, ¹H NMR (300 MHZ, CDCl₃) δ 8.46 (s, 1H), 7.40 (q, J=7.0 Hz, 2H), 7.25-7.20 (m, 1H), 7.03-6.92 (m, 1H), 6.85 (ddd, J=7.9, 2.0, 1.0 Hz, 1H), 3.09 (tt, J=7.6, 3.8 Hz, 1H), 1.06-0.96 (m, 2H), 0.83-0.72 (m, 2H) ppm.

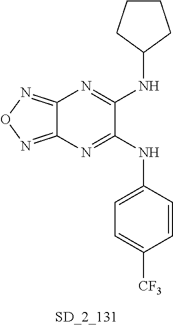 [SMILES: FC(F)(F)c1ccc(Nc2nc3nonc3nc2NC2CCCC2)cc1]

SD_2_131, yellow solid, 45% yield, ¹H NMR (300 MHz, CDCl₃) δ 8.50 (s, 1H), 7.78-7.68 (m, 2H), 7.11-7.05 (m, 2H), 4.50-4.51 (m, 1H), 2.27-2.13 (m, 2H), 1.88-1.71 (m, 4H), 1.66-1.54 (m, 2H) ppm.

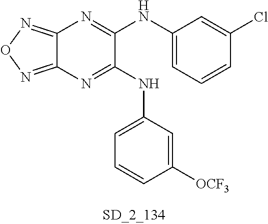 [SMILES: FC(F)(F)Oc1cccc(Nc2nc3nonc3nc2Nc2cccc(Cl)c2)c1]

SD_2_134, yellow solid, 43% yield, ¹H NMR (300 MHz, CDCl₃) δ 8.31 (s, 2H), 7.57-7.47 (m, 2H), 7.12 (ddt, J=8.3, 2.2, 1.0 Hz, 2H), 6.95-6.83 (m, 2H) ppm.

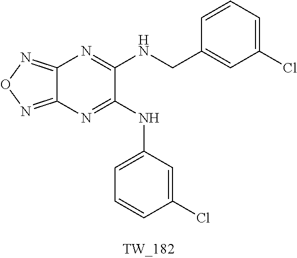 [SMILES: Clc1cccc(CNc2nc3nonc3nc2Nc2cccc(Cl)c2)c1]

TW_182, yellow solid, 20%, ¹H NMR (300 MHZ, (CD₃)₂CO) δ 7.81 (s, 1H), 7.58-7.26 (m, 6H), 7.20 (ddd, J=8.0, 2.1, 1.0 Hz, 1H) ppm.

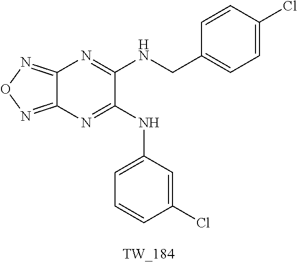 [SMILES: Clc1ccc(CNc2nc3nonc3nc2Nc2cccc(Cl)c2)cc1]

TW_184, yellow solid, 41%, ¹H NMR (300 MHz, (CD₃)₂CO) δ 7.80 (s, 1H), 7.58-7.45 (m, 3H), 7.48-7.30 (m, 3H), 7.19 (ddd, J=8.0, 2.1, 1.0 Hz, 1H), 4.84 (s, 2H) ppm.

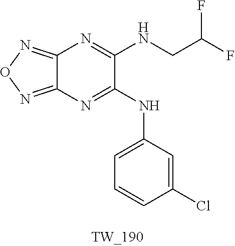 [SMILES: FC(F)CNc1nc2nonc2nc1Nc1cccc(Cl)c1]

TW_190, yellow solid, 28% yield, ¹H NMR (300 MHZ, CDCl₃) § 6.85 (ddd, J=7.9, 2.0, 1.0 Hz, 2H), 6.35-6.25 (m, 1H), 6.12 (dt, J=14.8, 4.1 Hz, 2H), 5.98-5.87 (m, 1H), 5.29 (s, 1H) ppm.

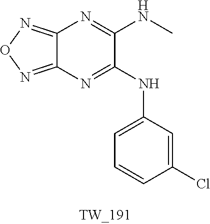 [SMILES: CNc1nc2nonc2nc1Nc1cccc(Cl)c1]

TW_191, yellow solid, 37% yield, ¹H NMR (300 MHz, CDCl₃) δ 7.98 (s, 1H), 7.68 (d, J=8.1 Hz, 1H), 7.44 (tdd, J=8.1, 4.0, 0.4 Hz, 2H), 7.21 (dddd, J=12.7, 8.0, 2.1, 1.0 Hz, 1H), 3.15 (s, 3H) ppm.

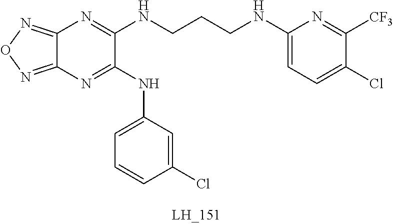 [SMILES: FC(F)(F)c1nc(NCCCNc2nc3nonc3nc2Nc2cccc(Cl)c2)ccc1Cl]

LH_151, yellow solid, 39% yield, ¹H NMR (300 MHz, CDCl₃) δ 8.84 (s, 1H), 7.57 (s, 1H), 7.45 (d, J=9 Hz, 1H), 7.34 (t, J=9 Hz, 1H), 7.14 (d, J=6 Hz, 1H), 6.94 (s, 1H), 6.82 (d, J=6 Hz, 1H), 6.59 (d, J=9 Hz, 1H), 5.34 (s, 1H), 3.72 (t, J=6 Hz, 2H), 3.53 (s, 2H), 2.02 (d, J=6 Hz, 2H) ppm.

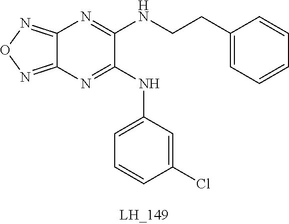 [SMILES: Clc1cccc(Nc2nc3nonc3nc2NCCc2ccccc2)c1]

LH_149, yellow solid, 40% yield, ¹H NMR (300 MHZ, CDCl₃) δ 8.35 (s, 1H), 7.34-7.43 (m, 8H), 7.21-7.30 (m, 4H), 6.95 (t, J=3 Hz, 1H), 6.83 (d, J=6 Hz, 1H), 3.90 (q, J=6 Hz, 2H), 3.05 (t, J=6 Hz, 2H) ppm.

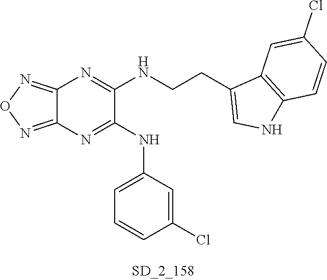 [SMILES: Clc1cccc(Nc2nc3nonc3nc2NCCc2c[nH]c3ccc(Cl)cc23)c1]

SD_2_158, yellow solid, 68% yield, ¹H NMR (300 MHZ, CDCl₃) δ 10.29 (s, 1H), 9.29 (s, 1H), 7.70-7.88 (m, 3H), 7.50 (s, 1H), 7.38-7.44 (m, 4H), 7.19-7.23 (m, 1H), 7.10-7.14 (m, 1H), 3.96 (t, J=6 Hz, 2H), 3.21 (t, J=6 Hz, 2H) ppm.

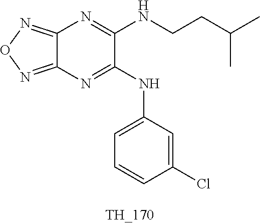 [SMILES: CC(C)CCNc1nc2nonc2nc1Nc1cccc(Cl)c1]

TH_170, yellow solid, 39% yield, ¹H NMR (300 MHz, CDCl₃) δ 8.62 (s, 1H), 7.38 (q, J=6.7 Hz, 2H), 7.21 (ddd, J=8.1, 2.0, 1.0 Hz, 1H), 6.98 (t, J=2.0 Hz, 1H), 6.86 (ddd, J=7.9, 2.0, 1.0 Hz, 1H), 3.69-3.47 (m, 2H), 1.72-1.74 (m, 2H), 1.69-1.53 (m, 2H), 1.00 (d, J=6.5 Hz, 6H) ppm.

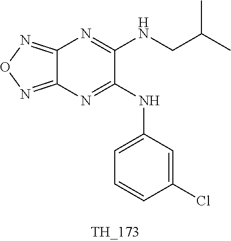 [SMILES: CC(C)CNc1nc2nonc2nc1Nc1cccc(Cl)c1]

TH_173, yellow solid, 41% yield, ¹H NMR (300 MHz, CDCl₃) 1H NMR (300 MHZ, CDCl₃) δ 8.81 (s, 1H), 7.37 (t, J=8.0 Hz, 1H), 7.19 (ddd, J=8.0, 2.0, 1.0 Hz, 1H), 6.98 (t, J=2.0 Hz, 1H), 6.86 (ddd, J=7.9, 2.0, 1.0 Hz, 1H), 3.44 (dd, J=7.0, 6.0 Hz, 2H), 2.12-1.97 (m, 1H), 1.04 (d, J=6.7 Hz, 6H) ppm.

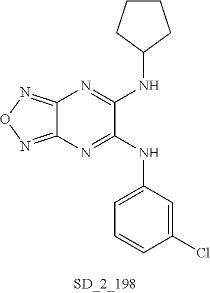 [SMILES: Clc1cccc(Nc2nc3nonc3nc2NC2CCCC2)c1]

SD_2_198, yellow solid, 45% yield, ¹H NMR (300 MHz, CDCl₃) δ 8.51 (s, 1H), 7.39 (t, J=8.0 Hz, 1H), 7.21 (d, J=7.7 Hz, 1H), 6.99 (s, 1H), 6.86 (d, J=7.9 Hz, 1H), 4.50-4.52 (m, 1H), 2.28-2.10 (m, 2H), 1.87-1.71 (m, 4H), 1.71-1.61 (m, 2H) ppm.

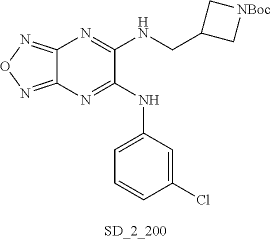 [SMILES: CC(C)(C)OC(=O)N1CC(CNc2nc3nonc3nc2Nc2cccc(Cl)c2)C1]

SD_2_200, yellow solid, 26% yield, ¹H NMR (300 MHZ, (CD₃)₂CO) δ 7.42 (td, J=8.0, 0.4 Hz, 1H), 7.22 (ddd, J=8.0, 2.1, 1.0 Hz, 1H), 3.98 (d, J=8.5 Hz, 2H), 3.85 (d, J=7.5 Hz, 2H), 3.72 (s, 2H), 3.09-2.94 (m, 1H), 1.41 (s, 9H) ppm

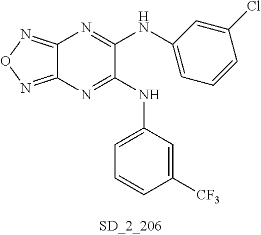 [SMILES: FC(F)(F)c1cccc(Nc2nc3nonc3nc2Nc2cccc(Cl)c2)c1]

SD_2_206, yellow solid, 46% yield, ¹H NMR (300 MHZ, (CD₃)₂CO) δ 9.98 (s, 1H), 7.87 (s, 2H), 7.63 (d, J=8.1 Hz, 2H), 7.52-7.43 (m, 2H), 7.25 (ddt, J=8.0, 2.1, 1.0 Hz, 1H) ppm.

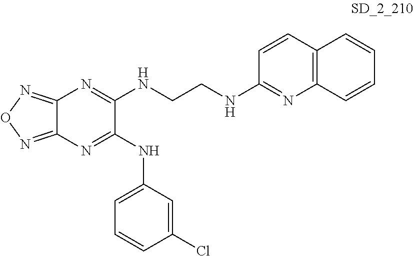 [SMILES: Clc1cccc(Nc2nc3nonc3nc2NCCNc2ccc3ccccc3n2)c1]

SD_2_210, yellow solid, 46% yield, ¹H NMR (300 MHz, (CD₃)₂CO) δ 8.76 (s, 1H), 7.89 (dd, J=8.9, 0.8 Hz, 1H), 7.68-7.55 (m, 2H), 7.32 (dd, J=15.6, 7.4 Hz, 2H), 7.20-7.09 (m, 2H), 6.84 (d, J=8.9 Hz, 1H), 6.79 (s, 1H), 3.93 (td, J=6.2, 3.5 Hz, 4H) ppm.

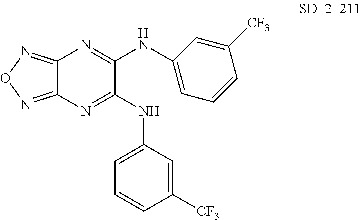 [SMILES: FC(F)(F)c1cccc(Nc2nc3nonc3nc2Nc2cccc(C(F)(F)F)c2)c1]

SD_2_211, yellow solid, 46% yield, ¹H NMR (300 MHZ, (CD₃)₂CO) δ 10.16 (s, 1H), 8.11 (s, 2H), 8.03 (d, J=8.3 Hz, 2H), 7.71 (ddt, J=8.1, 7.5, 0.7 Hz, 2H), 7.56 (dddd, J=7.8, 1.8, 1.4, 0.7 Hz, 2H) ppm.

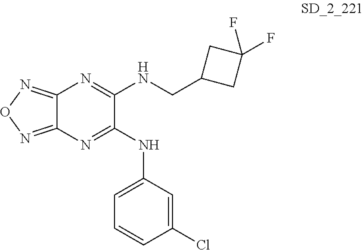 [SMILES: FC1(F)CC(CNc2nc3nonc3nc2Nc2cccc(Cl)c2)C1]

SD_2_221, yellow solid, 40% yield, ¹H NMR (300 MHz, CDCl₃) δ 8.49 (s, 1H), 7.51-7.31 (m, 2H), 7.22 (d, J=8.0 Hz, 1H), 6.99 (s, 1H), 6.86 (d, J=7.9 Hz, 1H), 3.77 (t, J=6.6 Hz, 2H), 2.88-2.78 (m, 1H) ppm.

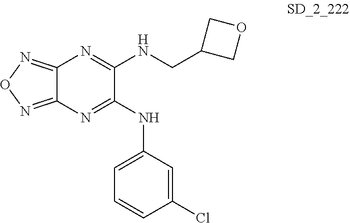 [SMILES: Clc1cccc(Nc2nc3nonc3nc2NCC2COC2)c1]

SD_2_222, yellow solid, 36% yield, ¹H NMR (300 MHZ, (CD₃)₂CO) δ 9.67 (s, 1H), 8.29 (t, J=2.0 Hz, 1H), 7.89 (ddd, J=8.3, 2.1, 0.9 Hz, 1H), 7.42 (d, J=8.1 Hz, 1H), 7.20 (ddd, J=8.0, 2.1, 0.9 Hz, 1H), 4.14 (ddd, J=12.4, 4.9, 2.3 Hz, 1H), 3.86-3.45 (m, 4H), 2.45-2.18 (m, 1H) ppm.

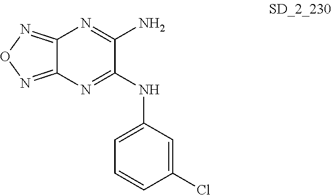 [SMILES: Nc1nc2nonc2nc1Nc1cccc(Cl)c1]

SD_2_230, yellow solid, 86% yield, ¹H NMR (300 MHZ, CD₃CN) δ 7.88 (s, 1H), 7.58 (s, 1H), 7.42 (t, J=9 Hz, 1H), 7.24 (dd, J=6 Hz, 1H), 6.63 (s, 1H) ppm.

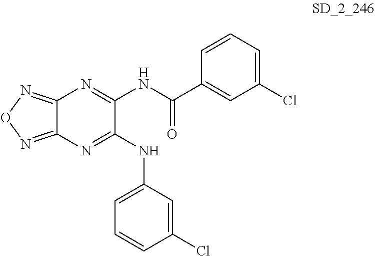 [SMILES: O=C(Nc1nc2nonc2nc1Nc1cccc(Cl)c1)c1cccc(Cl)c1]

SD_2_246, yellow solid, 20% yield, ¹H NMR (300 MHZ, CDCl₃) δ 9.56 (s, 1H), 8.28 (s, 1H), 8.22 (d, J=7.8 Hz, 1H), 8.10 (d, J=2.1 Hz, 1H), 7.92 (s, 1H), 7.89-7.81 (m, 1H), 7.75 (ddd, J=8.2, 2.2, 1.0 Hz, 1H), 7.66 (s, 1H), 7.35 (t, J=6 Hz, 1H) ppm.

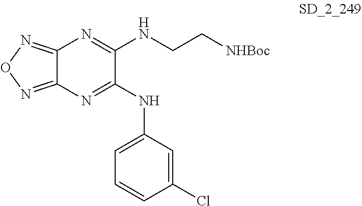 [SMILES: CC(C)(C)OC(=O)NCCNc1nc2nonc2nc1Nc1cccc(Cl)c1]

SD_2_249, yellow solid, 58% yield, ¹H NMR (300 MHZ, (CD₃)₂CO) δ 8.96 (s, 1H), 8.07 (s, 1H), 7.79 (d, J=8.2 Hz, 1H), 7.46 (t, J=8.1 Hz, 1H), 7.25 (d, J=8.1 Hz, 1H), 6.37 (s, 1H), 3.75 (t, J=5.9 Hz, 2H), 3.49 (t, J=6.1 Hz, 2H) 1.40 (s, 9H) ppm.

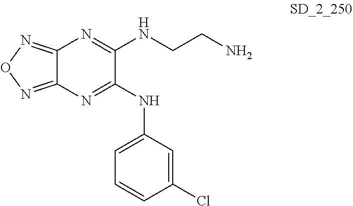 [SMILES: NCCNc1nc2nonc2nc1Nc1cccc(Cl)c1]

SD_2_250, yellow solid, 88.5% yield, It is TFA salt, ¹H NMR (300 MHZ, CD₃CN) δ 7.94 (s, 1H), 7.71 (d, J=8.1 Hz, 1H), 7.38 (t, J=8.1 Hz, 1H), 7.19 (dd, J=7.9, 1.9 Hz, 1H), 6.99 (s, 1H), 3.90 (t, J=5.9 Hz, 2H), 3.35 (t, J=6 Hz, 2H) ppm.

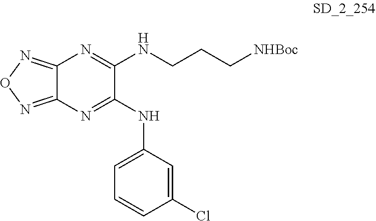 [SMILES: CC(C)(C)OC(=O)NCCCNc1nc2nonc2nc1Nc1cccc(Cl)c1]

SD_2_254, yellow solid, 67% yield, ¹H NMR (300 MHZ, (CD₃)₂CO) δ 7.87 (s, 1H), 7.45 (t, J=8.1 Hz, 1H), 7.28-7.17 (m, 1H), 6.29 (s, 1H), 3.70 (t, J=6.7 Hz, 2H), 3.28 (t, J=6.4 Hz, 2H), 1.91-1.93 (m, 2H), 1.43 (s, 9H) ppm.

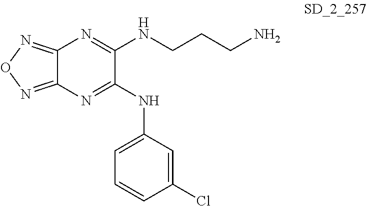 [SMILES: NCCCNc1nc2nonc2nc1Nc1cccc(Cl)c1]

SD_2_257, yellow solid, 67% yield, it is a TFA salt, ¹H NMR (300 MHZ, CD₃CN) δ 7.91 (s, 1H), 7.70-7.65 (m, 1H), 7.40 (td, J=8.1, 0.4 Hz, 1H), 7.22 (ddd, J=8.0, 2.1, 1.0 Hz, 1H), 7.04 (s, 1H), 3.76 (t, J=6.4 Hz, 2H), 3.23 (t, J=6.4 Hz, 2H), 2.21-2.09 (m, 2H) ppm.

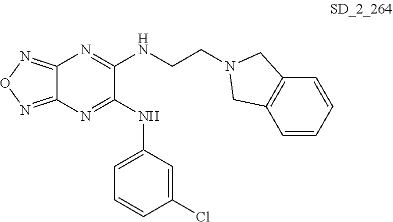 [SMILES: Clc1cccc(Nc2nc3nonc3nc2NCCN2Cc3ccccc3C2)c1]

SD_2_264, brown solid, 59% yield, ¹H NMR (300 MHz, CDCl₃) δ 8.05 (s, 1H), 7.29 (s, 1H), 7.25 (s, 1H), 7.22 (s, 1H), 7.20 (s, 4H), 7.11 (t, J=6.1 Hz, 1H), 7.05 (d, J=6.8 Hz, 1H), 3.99 (s, 4H), 3.76 (t, J=6.8 Hz, 2H), 3.05 (t, J=6.8 Hz, 2H) ppm.

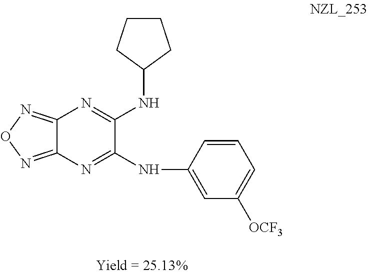 [SMILES: FC(F)(F)Oc1cccc(Nc2nc3nonc3nc2NC2CCCC2)c1]

NZL_253, yellow solid, 25.13% yield, ¹H NMR (300 MHZ, (CD₃)₂CO δ 7.75 (s, 1H), 7.63 (d, J=9.456 Hz, 1H), 7.56-7.50 (m, 1H), 7.14 (dtt, J=8.19 Hz, 1H), 4.54 (s, 1H), 2.23-2.09 (m, 2H), 1.83-1.62 (m, 6H) ppm.

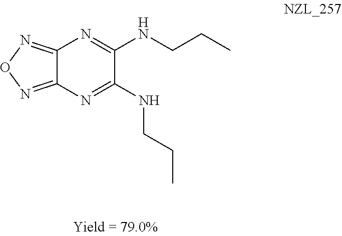 [SMILES: CCCNc1nc2nonc2nc1NCCC]

NZL_257, yellow solid, 79.0% yield, ¹H NMR (300 MHZ, (CD₃)₂CO) δ 7.31 (s, 2H), 3.56-3.49 (m, 4H), 1.76-1.62 (m, 5H), 1.00-0.94 (m, 8H) ppm.

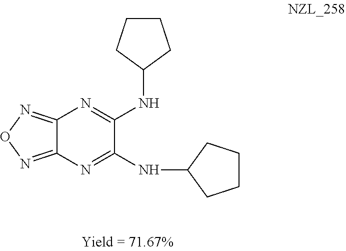 [SMILES: C1CCC(Nc2nc3nonc3nc2NC2CCCC2)C1]

NZL_258, yellow oil, 71.67% yield, ¹H NMR (300 MHz, (CD₃)₂CO) δ 7.20 (s, 2H), 4.66-4.45 (m, 2H), 2.024-2.03m, 4H), 1.83-1.45 (m, 12H) ppm.

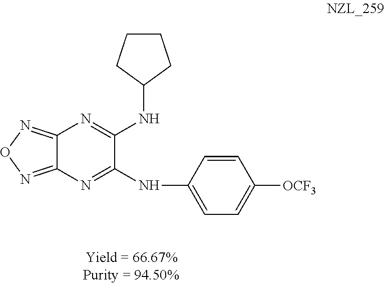 [SMILES: FC(F)(F)Oc1ccc(Nc2nc3nonc3nc2NC2CCCC2)cc1]

NZL_259, yellow solid, 66.67% yield, ¹H NMR (300 MHZ, (CD₃)₂CO) δ 7.81 (s, 1H), 7.54 (s, 1H), 7.40 (ddt, J=8.57 Hz 2H), 4.56 (m, 1H), 2.28-2.09 (m, 2H), 1.80-1.66 (m, 6H) ppm.

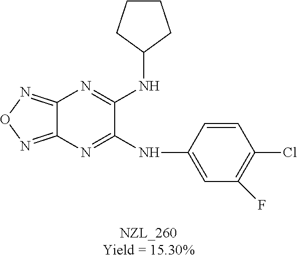 [SMILES: Fc1cc(Nc2nc3nonc3nc2NC2CCCC2)ccc1Cl]

NZL_260, yellow solid, 15.3% yield, 1H NMR (300 MHZ, (CD₃)₂CO) δ 7.80 (s, 1H), 7.53 (dd, J=8.50 Hz 1H), 7.42 (d, J=9.47 Hz, 1H), 4.55 (s, 1H), 2.21-2.07 (m, 2H), 1.82-1.64 (m, 6H) ppm.

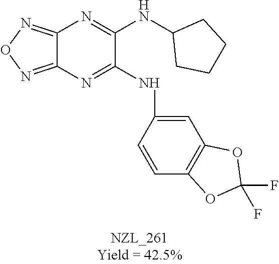 [SMILES: FC1(F)Oc2ccc(Nc3nc4nonc4nc3NC3CCCC3)cc2O1]

NZL_261, yellow solid, 42.5% yield, ¹H NMR (300 MHZ, (CD₃)₂CO) δ 7.70 (s, 1H), 7.53 (s, 1H), 7.35-7.271 (m, 2H), 4.55 (q, 1H), 2.21-2.08 (m, 2H), 1.82-1.64 (m, 6H) ppm.

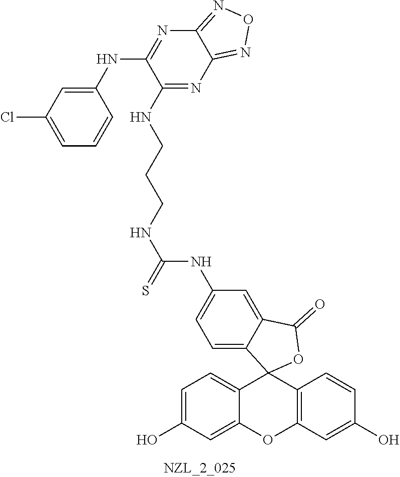 [SMILES: O=C1OC2(c3ccc(O)cc3Oc3cc(O)ccc32)c2ccc(NC(=S)NCCCNc3nc4nonc4nc3Nc3cccc(Cl)c3)cc21]

NZL_2_025, yellow gel, 18.3% yield, ¹H NMR (300 MHz, CD₃CN) δ 9.62 (s, 1H), 8.16-7.86 (m, 2H), 7.79-7.59 (m, 2H), 7.31-7.20 (m, 1H), 7.16-7.08 (m, 1H), 6.81-6.54 (m, 4H), 3.72-3.58 (m, 2H), 3.13 (q, 2H, J=8.40 Hz), 1.45-1.19 (m, 10H) ppm.

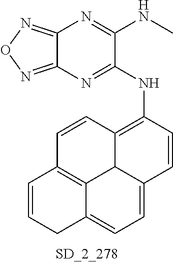 [SMILES: CNc1nc2nonc2nc1NC1=C2C=CC3=C4C(=CC=C(C=C1)C24)CC=C3]

SD_2_278, yellow solid, 30% yield, ¹H NMR (300 MHZ, (CD₃)₂CO)) δ 8.42 (s, 1H), 8.34-8.23 (m, 3H), 8.19-8.11 (m, 3H), 8.11-8.02 (m, 1H), 7.82 (s, 1H), 3.28 (d, J=4.9 Hz, 3H) ppm.

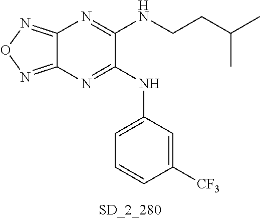 [SMILES: CC(C)CCNc1nc2nonc2nc1Nc1cccc(C(F)(F)F)c1]

SD_2_280, yellow solid, 45% yield, ¹H NMR (300 MHz, CDCl₃) δ 8.80 (s, 1H), 7.56 (t, J=7.9 Hz, 1H), 7.46 (d, J=7.7 Hz, 1H), 7.32 (s, 1H), 3.67-3.54 (m, 2H), 1.72 (dq, J=12.9, 6.5 Hz, 2H), 1.67-1.56 (m, 2H), 0.99 (d, J=6.4 Hz, 6H) ppm.

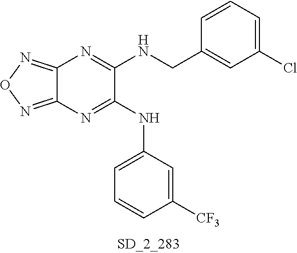 [SMILES: FC(F)(F)c1cccc(Nc2nc3nonc3nc2NCc2cccc(Cl)c2)c1]

SD_2_283,yellow solid, 48% yield, 1H NMR (300 MHZ, (CD₃)₂CO)) δ 8.06 (s, 2H), 7.73-7.62 (m, 1H), 7.57 (ddd, J=2.1, 1.6, 0.6 Hz, 1H), 7.54 (d, J=7.8 Hz, 1H), 7.48 (dddd, J=7.4, 2.2, 1.4, 0.6 Hz, 1H), 7.40 (td, J=7.7, 0.6 Hz, 1H), 7.36-7.31 (m, 1H), 4.90 (s, 2H) ppm.

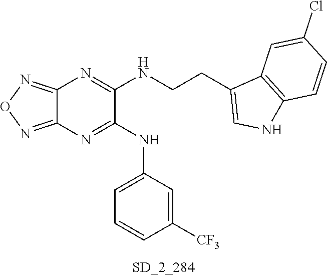 [SMILES: FC(F)(F)c1cccc(Nc2nc3nonc3nc2NCCc2c[nH]c3ccc(Cl)cc23)c1]

SD_2_284, brown solid, 58% yield, 1H NMR (300 MHZ, (CD₃)₂CO)) § 10.29 (s, 1H), 9.10 (s, 1H), 8.15 (d, J=13.2 Hz, 1H), 7.81 (s, 1H), 7.71-7.61 (m, 1H), 7.54 (d, J=7.9 Hz, 1H), 7.43 (dd, J=8.6, 0.6 Hz, 1H), 7.38 (s, 1H), 7.11 (ddd, J=8.6, 2.0, 0.4 Hz, 1H), 3.98 (t, J=7.1 Hz, 2H), 3.22 (td, J=7.1, 0.8 Hz, 2H) ppm.

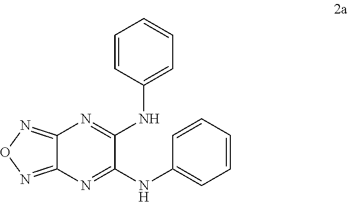 [SMILES: c1ccc(Nc2nc3nonc3nc2Nc2ccccc2)cc1]

SD_1_36 (2a), yellow solid, yield 46.8%, 1H NMR (300 MHZ, CD₃CN) δ 9.06 (s, 2H), 7.97-7.87 (m, 4H), 7.53-7.41 (m, 4H), 7.32-7.21 (m, 2H) ppm.

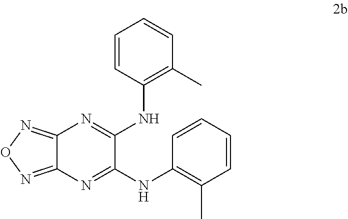 [SMILES: Cc1ccccc1Nc1nc2nonc2nc1Nc1ccccc1C]

SD_1_32 (2b), yellow solid, 49.2% yield, 1H NMR (300 MHZ, CDCl₃) δ 9.51 (s, 2H), 8.56 (dd, J=8.2, 1.3 Hz, 2H), 8.18 (s, 2H), 7.46-7.32 (m, 2H), 2.45 (s, 6H) ppm.

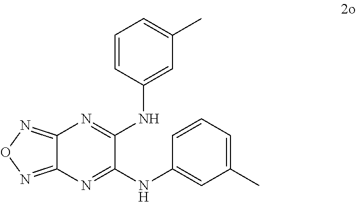 [SMILES: Cc1cccc(Nc2nc3nonc3nc2Nc2cccc(C)c2)c1]

SD_1_24 (2c), yellow solid, 44.6% yield, 1H NMR (300 MHz, (CD₃)₂CO) δ 9.52 (s, 2H), 7.64 (s, 3H), 7.33 (t, J=7.8 Hz, 3H), 7.16-6.94 (m, 2H), 2.38 (t, J=0.7 Hz, 6H) ppm.

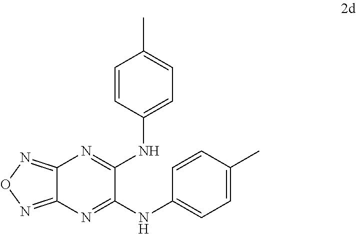 [SMILES: Cc1ccc(Nc2nc3nonc3nc2Nc2ccc(C)cc2)cc1]

SD_1_33 (2d), yellow solid, 45% yield, ¹H NMR (300 MHZ, Acetone) δ 8.11-7.85 (m, 4H), 7.28 (dq, J=8.0, 0.6 Hz, 4H), 2.36 (s, 6H) ppm.

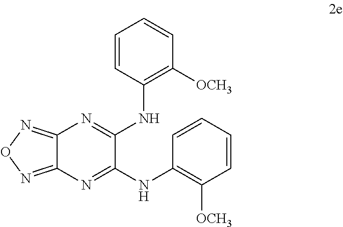 [SMILES: COc1ccccc1Nc1nc2nonc2nc1Nc1ccccc1OC]

SD_1_41 (2e), yellow solid, 15% yield, ¹H NMR (300 MHZ, (CD₃)₂CO) δ 9.03-8.64 (m, 2H), 7.28-6.97 (m, 8H), 3.92 (s, 6H) ppm.

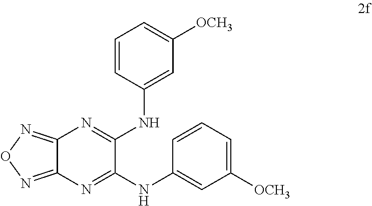 [SMILES: COc1cccc(Nc2nc3nonc3nc2Nc2cccc(OC)c2)c1]

SD_1_40 (2f), yellow solid, 26% yield, 1H NMR (300 MHZ, (CD₃)₂CO) δ 9.64 (s, 2H), 7.80-7.24 (brm, 4H), 7.34 (t, J=8.1 Hz, 2H), 6.80-6.75 (m, 2H), 3.82 (s, 6H) ppm.

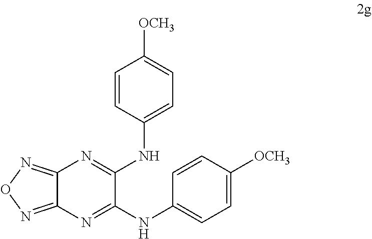 [SMILES: COc1ccc(Nc2nc3nonc3nc2Nc2ccc(OC)cc2)cc1]

SD_1_37 (2g), yellowsolid, yield 37%, ¹H NMR (300 MHz, CDCl₃) δ 9.28 (s, 1H), 8.26 (s, 1H), 7.88 (d, J=9.0 Hz, 2H), 7.06-6.98 (m, 4H), 3.87 (d, J=5.1 Hz, 6H) ppm.

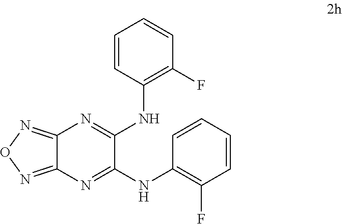 [SMILES: Fc1ccccc1Nc1nc2nonc2nc1Nc1ccccc1F]

SD_1_67 (2h), yellow solid, yield 49.66%, ¹H NMR (300 MHz, CD₃CN) δ 7.82 (s, 2H), 7.33 (d, J=0.7 Hz, 2H), 7.33-7.32 (m, 2H), 7.31-7.30 (m, 2H), 7.29 (d, J=1.8 Hz, 2H) ppm.

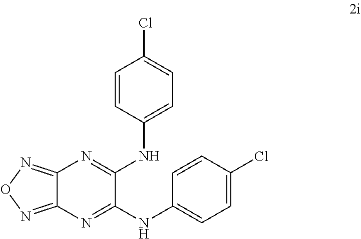 [SMILES: Clc1ccc(Nc2nc3nonc3nc2Nc2ccc(Cl)cc2)cc1]

SD_1_116 (2i), yellow solid, 50% yield, ¹HNMR (300 MHz, (CD₃)₂CO) δ 9.00 (s, 2H), 7.93-7.48 (m, 4H), 7.46 (d, J=8.76 Hz, 4H) ppm.

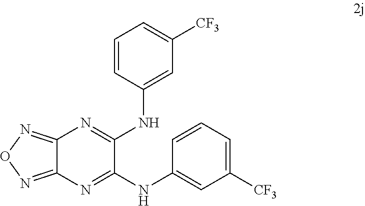 [SMILES: FC(F)(F)c1cccc(Nc2nc3nonc3nc2Nc2cccc(C(F)(F)F)c2)c1]

SD_2_211 (2j), yellow solid, 46% yield, ¹H NMR (300 MHZ, (CD₃)₂CO) δ 10.16 (s, 1H), 8.11 (s, 2H), 8.03 (d, J=8.3 Hz, 2H), 7.71 (ddt, J=8.1, 7.5, 0.7 Hz, 2H), 7.56 (dddd, J=7.8, 1.8, 1.4, 0.7 Hz, 2H) ppm.

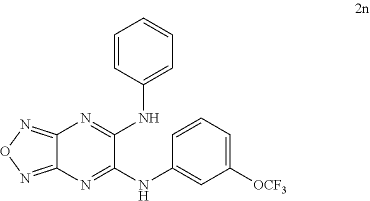 [SMILES: FC(F)(F)Oc1cccc(Nc2nc3nonc3nc2Nc2ccccc2)c1]

SD_1_83 (2n), yellow solid, 35% yield, 1H NMR (300 MHZ, CD₃CN) δ 8.99 (s, 1H), 7.67 (s, 1H), 7.57 (t, J=8.0 Hz, 2H), 7.54-7.47 (m, 2H), 7.31-7.26 (m, 2H), 7.19 (ddq, J=9.2, 2.2, 1.1 Hz, 1H) ppm.

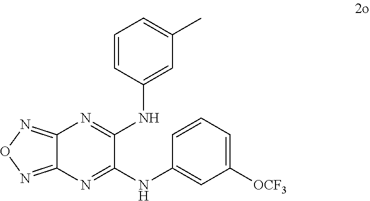 [SMILES: Cc1cccc(Nc2nc3nonc3nc2Nc2cccc(OC(F)(F)F)c2)c1]

SD_1_81 (20), yellow solid, 45% yield, 1H NMR (300 MHZ, CD₃CN) δ 9.01 (s, 1H), 7.62-7.52 (m, 2H), 7.46 (s, 2H), 7.40-7.33 (m, 1H), 7.23-7.15 (m, 1H), 7.10 (dtd, J=8.5, 1.4, 0.7 Hz, 1H), 2.41 (s, 3H) ppm.

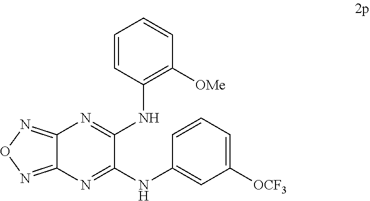 [SMILES: COc1ccccc1Nc1nc2nonc2nc1Nc1cccc(OC(F)(F)F)c1]

SD_1_48 (3j), yellow solid, 40% yield, 1H NMR (300 MHZ, (CD₃)₂CO) δ 9.64 (s, 1H), 8.25-8.13 (m, 2H), 7.56-7.45 (m, 2H) ppm.

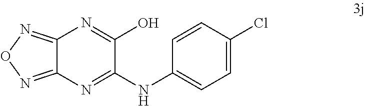 [SMILES: Oc1nc2nonc2nc1Nc1ccc(Cl)cc1]

SD_1_82 (2p), yellow solid, 40% yield, ¹H NMR (300 MHZ, CD₃CN) δ 9.38 (s, 1H), 8.41 (s, 1H), 7.63-7.54 (m, 1H), 7.32 (s, 1H), 7.28-7.23 (m, 1H), 7.22-7.16 (m, 1H), 7.15-7.12 (m, 1H), 7.12-7.07 (m, 1H), 3.94 (s, 3H) ppm.

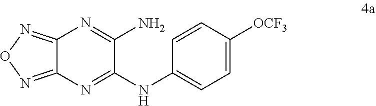 [SMILES: Nc1nc2nonc2nc1Nc1ccc(OC(F)(F)F)cc1]

SD_1_62 (4a), yellow solid, 48% yield, ¹H NMR (300 MHZ, CD₃CN) δ 8.95 (s, 1H), 8.06-7.90 (m, 2H), 7.53-7.35 (m, 4H), 4.25 (s, 2H) ppm.

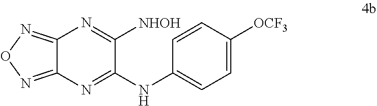

SD_1_51 (4b), yellow solid, 30% yield, ¹H NMR (300 MHZ, (CD₃)₂CO) δ 9.23 (s, 1H), 8.24 (d, J=9.0 Hz, 2H), 8.17 (d, J=8.5 Hz, 2H), 5.13 (s, 1H), 4.25 (s, 1H) ppm.

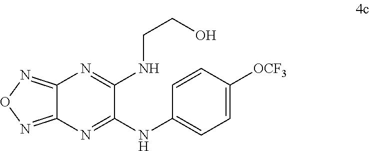 [SMILES: OCCNc1nc2nonc2nc1Nc1ccc(OC(F)(F)F)cc1]

SD_1_52 (4c), yellow solid, 40% yield, ¹H NMR (300 MHz, (CD₃)₂CO) δ 8.12-7.46 (m, 2H), 7.39 (dtd, J=8.3, 2.2, 1.2 Hz, 1H), 3.86 (ddd, J=6.2, 5.2, 1.1 Hz, 1H), 3.81-3.71 (m, 1H) ppm.

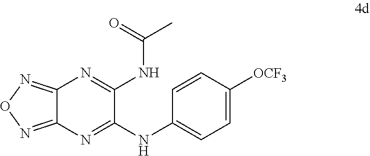 [SMILES: CC(=O)Nc1nc2nonc2nc1Nc1ccc(OC(F)(F)F)cc1]

SD_1_74 (4d), yellow solid, 35% yield, ¹H NMR (300 MHZ, (CD₃)₂CO) δ 9.74 (s, 1H), 8.59 (d, J=4.9 Hz, 2H), 8.23-8.15 (m, 2H), 4.25 (s, 3H) ppm.

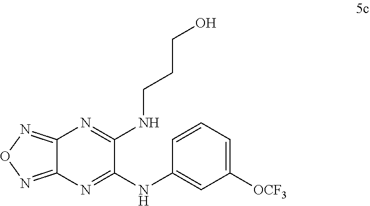 [SMILES: OCCCNc1nc2nonc2nc1Nc1cccc(OC(F)(F)F)c1]

SD_1_72 (5c), yellowsolid, yield 25%, ¹H NMR (300 MHZ, (CD₃)₂CO)) δ 9.16 (s, 1H), 8.06-7.57 (m, 2H), 7.41 (d, J=8.5 Hz, 2H), 3.74 (dt, J=8.9, 5.9 Hz, 4H), 2.01-1.89 (m, 2H), 1.30 (s, 1H) ppm.

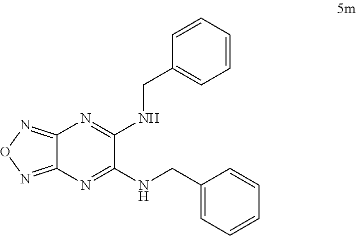 [SMILES: c1ccc(CNc2nc3nonc3nc2NCc2ccccc2)cc1]

SD_1_89 (5m), yellow solid, 48% yield, ¹H NMR (300 MHZ, CD₃CN) δ 7.45 (tt, J=1.7, 0.6 Hz, 1H), 7.43 (ddd, J=1.9, 1.1, 0.5 Hz, 3H), 7.45-7.37 (m, 2H), 7.43-7.35 (m, 1H), 7.40-7.33 (m, 1H), 7.38-7.30 (m, 1H), 7.36-7.29 (m, 1H), 6.92 (s, 2H), 4.73 (s, 4H) ppm.

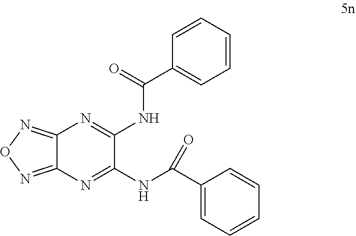 [SMILES: O=C(Nc1nc2nonc2nc1NC(=O)c1ccccc1)c1ccccc1]

SD_1_93 (5n), yellow solid, 27% yield, ¹H NMR (300 MHZ, CD₃CN) δ 7.96-7.90 (m, 2H), 7.70-7.63 (m, 2H), 7.59-7.54 (m, 4H) ppm.

### Example 11: Materials and Methods

General Methods. MDR strains E. coli AR-0493, AR-0346, and AR-0538, Salmonella Enteritidis AR-0496, Salmonella Typhimurium AR-0635, K. pneumoniae AR-0497, A. baumannii AR-0300 and AR-0310 were all obtained from the CDC & FDA Antibiotic Resistance Isolate Bank. E. coli K-12 was a gift from the Copley lab (Department of MCD Biology, University of Colorado Boulder). HeLa cells were purchased from the ATCC. All bacterial strains were grown on trypticase soy agar supplemented with 5% sheep blood (BAP). Bacteria were then cultured in Luria-Bertani (LB) medium at 35° C. until they achieve the log phase before screening or antimicrobial testing using CAMHB media. MIC, MRC, and checkerboard analyses and mammalian growth inhibition analyses in Hela cells were performed as described previously. More details are given in Supporting information.

Compound Screening. The TimTec fragment-based library was screened using a modified broth microdilution assay. The MDR E. coli strain AR-0493 was used to screen the fragment-based library for RMA activity. 96-well assay plates were prepared containing 50 μL CAMHB supplemented with 4 μg/mL of colistin. 500 nL of each compound (15 mM in DMSO) was pinned to the assay plate using the CyBi-Well 96-channel simultaneous pipettor (Cybio). These plates were inoculated with 50 μL E. coli AR-0493 diluted in CAHMB to OD₆₀₀ 0.002. The final concentration of colistin in the screen was 2 μg/mL, the final concentration of each screening compound was 75 μM, and the final inoculum concentration was OD₆₀₀ 0.001. All plates were incubated at 35° C. with shaking for 19 hours before results were interpreted.

TABLE 1
Compound 3 selectively potentiates polymyxins in multidrug-resistant
E. coli.

| Antibiotics | MIC^{a} | MIC (+3) ^{a,b} | fold of potentiation | INT^{a,c} |
|---|---|---|---|---|
| Colistin | 16 | 1 | 16 | R |
| Polymyxin B | 16 | 1 | 16 | R |
| Ceftazidime | >64 | >64 | — | R |
| Meropenem | 0.25 | 0.25 | — | S |
| Azithromycin | >64 | >64 | — | R |
| Ciprofloxacin | 32 | 32 | — | R |
| Rifampicin | 16 | 16 | — | R |
| Tetracycline | >64 | >64 | — | R |
| Gentamicin | 0.5 | 0.5 | — | S |

^{a}MIC values are in μg/mL.
^{b} MIC in the presence of 1 μg/mL of compound 3.
^{c}S-R interpretation (INT) derived from CLSI 2021, M100, Ed 31.

TABLE 2
Colistin MICs in the presence and absence of compound 3 in a panel of
Gram-negative bacteria.

| | Resistance | Colistin | Colistin | Folds of |
|---|---|---|---|---|
| Bacterial strain | mechanism | MIC^{a} | MIC (+3)^{b} | potentiation |
| E. coli AR-0493 | mcr-1 | 16 | 1 | 16 |
| E. coli AR-0346 | mcr-1 | 8 | 0.5 | 16 |
| E. coli AR-0538 | mcr-2 | 8 | 0.5 | 16 |
| Salmonella Enteritidis | mcr-1 | 16 | 1 | 16 |
| AR-0496 | | | | |
| Salmonella Typhimurium | mcr-4 | 16 | 1^{c} | 16 |
| AR-0635 | | | | |
| K. pneumoniae AR-0497 | mcr-1 | 8 | 0.5 | 16 |
| A. baumannii AR-0300 | | 8 | 0.25 | 32 |
| A. baumannii AR-0310 | | 16 | 0.5 | 32 |
| P. aeruginosa AR-0768 | | 16 | 4 | 4 |
| E. coli K-12 | | 0.25 | 0.25 | — |

^{a}Colistin MIC in μg/mL.
^{b}Colistin MIC in μg/mL, in the presence of 1 μg/mL of compound 3, unless otherwise noted.
^{c}Colistin MIC in μg/mL, in the presence of 2 μg/mL of compound 3.

TABLE 3
MRC Tests of compounds in AR-0493
(Compound + 2 μg/mL Colistin)

| Compounds | MRC | |
|---|---|---|
| 2a | 0.5 | μg/mL |
| 2b | > 128 | μg/mL |
| 2c | 0.5 | μg/mL |
| 2d | 0.5 | μg/mL |
| 2e | >16 | μg/mL |
| 2f | 4 | μg/mL |
| 2g | >16 | μg/mL |
| 2h | 4 | μg/mL |
| 2i | 0.25 | μg/mL |
| 2j | 2 | μg/mL |
| 2k | 0.125-0.25 | μg/mL |
| 2l | >16 | μg/mL |
| 2m | 0.25 | μg/mL |
| 3a | >16 | μg/mL |
| 3b | >16 | μg/mL |
| 3d | 64 | μg/mL |
| 3e | 4 | μg/mL |
| 3f | 16 | μg/mL |
| 3g | >16 | μg/mL |
| 3h | 16 | μg/mL |
| 3i | 8 | μg/mL |
| 3j | >16 | μg/mL |
| 3k | >16 | μg/mL |
| 3l | >16 | μg/mL |
| 3m | >16 | μg/mL |
| 3n | 8 | μg/mL |
| 3p | >16 | μg/mL |
| 3q | >16 | μg/mL |
| 3r | 4 | μg/mL |
| 3s | >16 | μg/mL |
| 4a | >16 | μg/mL |
| 4b | >16 | μg/mL |
| 4c | 4 | μg/mL |
| 4d | 16 | μg/mL |
| 5a | 8 | μg/mL |
| 5b | 8 | μg/mL |
| 5c | 8 | μg/mL |
| 5i | 8 | μg/mL |

TABLE 4
Additional MRC Tests of analog compounds
MRC tests in AR0493

| Compounds | MRC* | |
|---|---|---|
| SD_2_108 | 8 | μg/mL |
| SD_2_109 | 0.5 | μg/mL |
| SD_2_131 | 0.25 | μg/mL |
| SD_2_132 | 1 | μg/mL |
| TW-184 | 0.5 | μg/mL |
| TW-190 | 2 | μg/mL |
| LH-149 | 0.5 | μg/mL |
| LH-151 | 0.5 | μg/mL |
| SD_2_200 | 4 | μg/mL |
| SD_2_246 | 4 | μg/mL |
| SD_2_249 | 4 | μg/mL |
| SD_2_256 | >16 | μg/mL |
| SD_2_264 | 2 | μg/mL |
| NZL-Fluorescence | 16 | μg/mL |

Colistin MIC in AR0493: 16 μg/mL;
*minimum re-sensitizing concentration in the presence of 2 μg/mL Colistin

TABLE 5
Biological evaluation of the bisanilino analogs of 7a-s

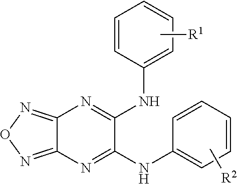

| Entry | R¹ | R² | MRC^{a} | MIC^{b} | GI₅₀^{c} | SI^{d} |
|---|---|---|---|---|---|---|
| 7a | H | H | 0.5 | >64 | 4.4 | 8.8 |
| 7b | o-Me | o-Me | 8 | — | — | — |
| 7c (1) | m-Me | m-Me | 0.5 | >64 | 1.5 | 30 |
| 7d | p-Me | p-Me | 0.5 | >64 | 1.1 | 22 |
| 7e | o-OMe | o-OMe | >64 | — | — | — |
| 7f | m-OMe | m-OMe | 2 | — | — | — |
| 7g | p-OMe | p-OMe | >64 | — | — | — |
| 7h | o-F | o-F | 4 | — | — | — |
| 7i | m-F | m-F | 0.5 | >64 | 20 | 40 |
| 7j | p-F | p-F | 0.5 | >64 | 9.4 | 19 |
| 7k | o-Cl | o-Cl | 8 | — | — | — |
| 7l | m-Cl | m-Cl | 0.25 | >64 | 1.1 | 44 |
| 7m | p-Cl | p-Cl | 0.5 | >64 | 2.8 | 56 |
| 7n | o-OCF₃ | o-OCF₃ | >64 | — | — | — |
| 7o | m-OCF₃ | m-OCF₃ | 0.25 | >64 | 43 | 17 |
| 7p | p-OCF₃ | p-OCF₃ | 0.25 | >64 | 5.7 | 23 |
| 7q | m-OCF₃ | H | 1 | — | — | — |
| 7r | m-OCF₃ | m-Me | 0.5 | >64 | 12 | 24 |
| 7s | m-OCF₃ | o-OMe | >64 | — | — | — |

—Not tested.
^{a}MRC values are in μg/mL in the presence of 2 μg/ml of colistin.
^{b}MIC values are in μg/mL.
^{c}GI₅₀ values are half maximum inhibitory concentrations in μg/mL.
^{d}SI (Selective Index) indicates the ratio of GI₅₀ and MRC values.

TABLE 6
Biological evaluation of the monoanilino analogs 8a-p.

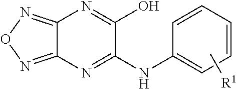

| Entry | R¹ | MRC^{a} | MIC^{b} | GI₅₀^{c} | SI^{d} |
|---|---|---|---|---|---|
| 8a | H | >64 | — | — | — |
| 8b | o-Me | 64 | — | — | — |
| 8c (2) | m-Me | 8 | — | — | — |
| 8d | p-Me | 64 | — | — | — |
| 8e | o-OMe | 4 | >64 | >100 | >24 |
| 8f | m-OMe | 16 | — | — | — |
| 8g | p-OMe | >64 | — | — | — |
| 8h | o-F | 64 | — | — | — |
| 8i | m-F | 64 | — | — | — |
| 8j | p-F | 32 | — | — | — |
| 8k | o-Cl | 16 | — | — | — |
| 8l | m-Cl | 16 | — | — | — |
| 8m | p-Cl | 32 | — | — | — |
| 8n | o-OCF₃ | >64 | — | — | — |
| 8o | m-OCF₃ | 4 | >64 | 24 | 6 |
| 8p | p-OCF₃ | 16 | — | — | — |

—Not tested.
^{a}MRC values are in μg/mL in the presence of 2 μg/ml of colistin.
^{b}MIC values are in μg/mL.
^{c}GI₅₀ values are half maximum inhibitory concentrations in μg/mL.
^{d}SI (Selective Index) is the ratio of GI₅₀ and MRC values.

TABLE 7
MICs of colistin in the absence or presence of two best RMAs in
various MCR bacterial strains.

| | Resistance | Colistin MIC [symbol] | | |
|---|---|---|---|---|
| Bacterial strain | gene | [symbol] | ( [symbol] ) [symbol] | ( [symbol] ) [symbol] |
| E. coli AR-0493 | mcr-1 | 8 | 1 | 1 |
| E. coli AR-0538 | mcr-2 | 8 | 1 | 2 |
| S. Typhimurium AR-0 [symbol] 39 | mcr-3 | 8 | 1 | 2 |
| S. Typhimurium AR-0635 | mcr-4 | 32 | 1 | 1 |
| K. pneumoniae AR-0497 | mcr-1 | 8 | 1 | 1 |
| A. baumannii AR-0310 | | 32 | 0.25 | 0.25 |
| P. aeruginosa AR-0239 | | 4 | 1 | 1 |
| P. aeruginosa AR-0257 | | 8 | 2 | 2 |

^{a}Colistin MIC in μg/mL.
^{b}Colistin MIC in μg/mL, in the presence of 1 μg/mL of compound indicated, unless otherwise noted. [symbol]
indicates data missing or illegible when filed

### REFERENCES

1. Laxminarayan, R.; Duse, A.; Wattal, C.; Zaidi, A. K. M.; Wertheim, H. F. L.; Sumpradit, N.; Vlieghe, E.; Hara, G. L.; Gould, I. M.; Goossens, H.; Greko, C.; So, A. D.; Bigdeli, M.; Tomson, G.; Woodhouse, W.; Ombaka, E.; Peralta, A. Q.; Qamar, F. N.; Mir, F.; Kariuki, S.; Bhutta, Z. A.; Coates, A.; Bergstrom, R.; Wright, G. D.; Brown, E. D.; Cars, O. Antibiotic resistance—the need for global solutions. Lancet Infect. Dis. 2013, 13 (12), 1057-1098.
2. CDC. Antibiotic resistance threats in the United States, 2019. Atlanta, GA: U.S. Department of Health and Human Services, CDC; 2019.
3 O'Neill, J. (Chaired) (2014 December) Antimicrobial resistance: tackling a crisis for the health and wealth of nations The Review on Antimicrobial Resistance.
4. Ferri, M.; Ranucci, E.; Romagnoli, P.; Giaccone, V. Antimicrobial resistance: A global emerging threat to public health systems. Crit. Rev. Food Sci. Nutr. 2017, 57 (13), 2857-2876.
5. Li, J.; Nation, R. L.; Turnidge, J. D.; Milne, R. W.; Coulthard, K.; Rayner, C. R.; Paterson, D. L. Colistin: the re-emerging antibiotic for multidrug-resistant Gram-negative bacterial infections. Lancet Infect. Dis. 2006, 6 (9), 589-601.
6. Kwon, K. H.; Oh, J. Y.; Yoon, Y. S.; Jeong, Y. J.; Kim, K. S.; Shin, S. J.; Chung, J. W.; Huh, H. J.; Chae, S. L., Park, S. Y. Colistin treatment in carbapenem-resistant Acinetobacter baumannii pneumonia patients: Incidence of nephrotoxicity and outcomes. Int. J. Antimicrob. Ag. 2015, 45 (6), 605-609.
7. Qureshi, Z. A.; Hittle, L. E.; O'Hara, J. A., Rivera, J. I., Syed, A., Shields, R. K., Pasculle, A. W., Ernst, R. K., Doi, Y. Colistin-resistant Acinetobacter baumannii: Beyond carbapenem resistance. Clin. Infect. Dis. 2015, 60 (9), 1295-1303.
8 Michalopoulos, A. S.; Tsiodras, S.; Rellos, K.; Mentzelopoulos, S.; Falagas, M. E. Colistin treatment in patients with ICU-acquired infections caused by multiresistant Gram-negative bacteria: The renaissance of an old antibiotic. Clin. Microbiol. Infect. 2005, 11 (2), 115-121.
9. WHO Advisory Group on Integrated Surveillance of Antimicrobial Resistance (AGISAR), Critically Important Antimicrobials for Human Medicine (2019), 6th Revision.
10. Rashid, R; Veleba, M; Kline, K. A. Focal Targeting of the Bacterial Envelope by Antimicrobial Peptides. Front. Cell Dev. Biol. 2016, 4, 55.
11. Needham, B. D.; Trent, M. S. Fortifying the barrier: the impact of lipid A remodelling on bacterial pathogenesis. Nat. Rev. Microbiol. 2013, 11 (7), 467-81.
12. Olaitan, A. O.; Morand, S.; Rolain, J. M. Mechanisms of polymyxin resistance: acquired and intrinsic resistance in bacteria. Front. Microbiol. 2014, 5, 643.
13. Nikaido, H.; Molecular basis of bacterial outer membrane permeability revisited. Microbiol. Mol. Biol. Rev. 2003, 67 (4), 593-656.
14. Guo, L.; Lim, K. B.; Poduje, C. M.; Daniel, M.; Gunn, J. S.; Hackett, M.; Miller, S. I. Lipid A acylation and bacterial resistance against vertebrate antimicrobial peptides. Cell 1998, 95(2), 189-198.
15. Ernst, R. K.; Yi, E. C.; Guo, L.; Lim, K. B.; Burns, J. L.; Hackett, M.; Miller, S. I. Specific lipopolysaccharide found in cystic fibrosis airway Pseudomonas aeruginosa. Science 1999, 286 (5444), 1561-5.
16. Nang, S. C.; Li. J.; Velkov, T. The rise and spread of mcr plasmid-mediated polymyxin resistance. Crit. Rev. Microbiol. 2019, 45 (2), 131-161.
17. Wang, Y.; Tian, G. B.; Zhang, R.; Shen, Y.; Tyrrell, J. M.; Huang, X.; Zhou, H.; Lei, L.; Li, H. Y.; Doi, Y.; Fang, Y.; Ren, H.; Zhong, L. L.; Shen, Z.; Zeng, K. J.; Wang, S.; Liu, J. H.; Wu, C.; Walsh, T. R.; Shen, J. Prevalence, risk factors, outcomes, and molecular epidemiology of mcr-1-positive Enterobacteriaceae in patients and healthy adults from China: an epidemiological and clinical study. Lancet Infect. Dis. 2017, 17 (4), 390-399.
18. Li, X.; Plésiat, P.; Nikaido, H. The challenge of efflux-mediated antibiotic resistance in Gram-negative bacteria Clin. Microbiol. Rev. 2015, 28 (2), 337-418. B) Yu, M.; Chua, S. L. Demolishing the great wall of biofilms in Gram-negative bacteria: To disrupt or disperse? Med. Res. Rev. 2020, 40, 1103-1116.
19. Brown, P.; Dawson, M. J. Development of new polymyxin derivatives for multi-drug resistant Gram-negative infections. J. Antibiot. 2017, 70, 386-394.
20. Vaara, M Polymyxins and their potential next generation as therapeutic antibiotics. Front. Microbiol. 2019b, 10, 1689.
21 Velkov, T. and Roberts KD (2019) Discovery of novel polymyxin-like antibiotics, in Polymyxin antibiotics: From laboratory bench to bedside (Li J, Nation R L, and Kaye K eds) pp 343-362, Springer International Publishing, New York.
22. Clancy, C. J.; Chen, L.; Hong, J. H.; Cheng, S.; Hao, B.; Shields, R. K.; Farrell, A. N.; Doi, Y.; Zhao, Y.; Perlin, D. S. Mutations of the ompK36 porin gene and promoter impact responses of sequence type 258, KPC-2-producing Klebsiella pneumoniae strains to doripenem and doripenem-colistin. Antimicrob. Agents Ch. 2013, 57, 5258-5265.
23. Gaibani, P.; Lombardo, D.; Lewis, R. E.; Mercuri, M.; Bonora, S.; Landini, M. P. and Ambretti, S. In vitro activity and post-antibiotic effects of colistin in combination with other antimicrobials against colistin-resistant KPC-producing Klebsiella pneumoniae bloodstream isolates. J. Antimicrob. Chemother. 2014, 69, 1856-1865.
24. Betts, J. W.; Phee, L. M.; Hornsey, M.; Woodford, N. and Wareham, D. W. In vitro and in vivo activities of tigecycline-colistin combination therapies against carbapenem-resistant Enterobacteriaceae. Antimicrob. Agents Ch. 2014a, 58, 3541-3546.
25. Walsh, C. C.; Landersdorfer, C. B.; McIntosh, M. P.; Peleg, A. Y.; Hirsch, E. B.; Kirkpatrick, C. M.; Bergen, P. J. Clinically relevant concentrations of fosfomycin combined with polymyxin B, tobramycin or ciprofloxacin enhance bacterial killing of Pseudomonas aeruginosa, but do not suppress the emergence of fosfomycin resistance. J. Antimicrob. Chemother. 2016, 71, 2218-2229.
26. Barkera, W. T.; Chandlerb, C. E.; Melandera, R. J.; Ernstb, R. K.; Melander, C. Tryptamine derivatives disarm colistin resistance in polymyxin-resistant Gram-negative bacteria. Bioorg. Med. Chem. 2019, 27, 1776-1788.
27. Lan, X.; Yan, H.; Lin, F.; Hou, S.; Li, C.; Wang, G.; Sun, W.; Xiao, J.; Li, S. Design, Synthesis and biological evaluation of 1-phenyl-2-(phenylamino) ethanone derivatives as novel mcr-1 inhibitors. Molecules 2019, 24, 2719.
28. Harris, T. L.; Worthington, R. J.; Hittle, L. E.; Zurawski, D. V.; Ernst, R. K.; Melander, C. Small molecule downregulation of PmrAB reverses lipid A modification and breaks colistin resistance. ACS Chem. Biol. 2014, 9, 122-127.
29. Wang, X.; Chen, J.; Wang, W.; Jaunaraj, A.; Wang, X. Tryptoline-based benzothiazoles re-sensitize MRSA to B-lactam antibiotics. Bioorg. Med. Chem. 2019, 27, 115095-115106. B) He, W.; Griffiths, B. M.; Wang, W.; Wang, X. Diastereoselective synthesis and biological evaluation of enantiomerically pure tricyclic indolines. Org. Biomol. Chem., 2017, 15, 4241-4245. C) Zhu, Y.; Cleaver, L.; Wang, W.; Podoll, J. D.; Walls, S.; Jolly, A.; Wang, X. Tetracyclic indolines as a novel class of B-lactam-selective resistance-modifying agent for MRSA. Eur. J. Med. Chem. 2017, 125, 130-142. D) Barbour, P. M.; Wang, W.; Chang, L.; Pickard, K. L.; Rais, R.; Slusher, B. S.; Wang, X. Property-guided synthesis of aza-tricyclic indolines: development of gold catalysis en route. Adv. Synth. Catal. 2016, 358, 1482-1490. E) Podoll, J. D.; Liu, Y.; Chang, L.; Walls, S.; Wang, W.; Wang, X. Bio-inspired synthesis yields a tricyclic indoline that selectively re-sensitizes MRSA to B-lactam antibiotics. Proc. Natl. Acad. Sci. USA. 2013, 110, 15573-15578.
30 CLSI M100 Performance Standards for Antimicrobial Susceptibility Testing, 31st Ed., 2021.
31. Li, Q. Application of fragment-based drug discovery to versatile targets. Front. Mol. Biosci. 2020, 7, 180.
32. Humphries, R. M.; Abbott, A. N.; Hindlerd. J. A. Understanding and addressing CLSI breakpoint revisions: A primer for clinical laboratories. J. Clin. Microbiol. 2019, 57(6), e00203-19.
33. Childress, E. S.; Salamoun, J. M.; Hargett, S. R.; Alexopoulos, S. J.; Chen, S.; Shah, D. P.; Santiago-Rivera, J.; Garcia, C. J.; Dai, Y.; Tucker, S. P.; Hoehn, K. L.; Santos, W. L. [1,2,5]Oxadiazolo[3,4-b]pyrazine-5,6-diamine derivatives as mitochondrial uncouplers for the potential treatment of nonalcoholic steatohepatitis. J. Med. Chem. 2020, 63, 2511-2526.
34 Salamoun, J. M.; Garcia, Ch. J.; Hargett, S. R.; Murray, J. H.; Chen, S.; Beretta, M.; Alexopoulos, S. J.; Shah, D. P.; Olzomer, E. M.; Tucker, S. P.; Hoehn, K. L.; Santos, W. L. 6-Amino [1,2,5] oxadiazolo-[3,4-b]pyrazin-5-ol derivatives as efficacious mitochondrial uncouplers in STAM mouse model of nonalcoholic steatohepatitis. J. Med. Chem. 2020, 63, 6203-6224.
35 Sopirala, M. M.; Mangino, J. E.; gebreyes, W. A.; Beller, B.; Bannerman, T.; Balada-Llasat, J. M.; Pancholi, P. Synergy testing by Etest, microdilution checkerboard, and time-kill methods for pan-drug-resistant Acinetobacter baumannii. Antimicrob. Agents Chemother. 2010, 54, 4678-4683.

1. A resistance-modifying agent (RMA) according to the compound of Formula I: 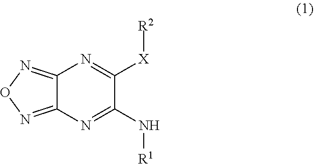
or a stereoisomer, pharmaceutically acceptable salt thereof, wherein,
X is —H, —OH, —S, —NH—, —NH₂, —NHCO—, —N(OH)—, or —NHOH;
R₂ is H, aryl, aryl halide, alkyl, cycloalkane, heteroalkyl, alkyl halide, aromatic, heteroaromatic, amino, amine, heterocycloalkane, cycloalkane, fused aromatic or fused heteroaromatic, all of the foregoing being substituted or unsubstituted, (CN)Ph-, (NO₂)Ph-, (CF₃)Ph-, —C(═O)R₃, —C(═)OOR₃, —S(═O)R₃, —S(═O)₂R₃, —S(═O)₂NHR₃, —C(═O)R₃, —C(═)OOR₃, —S(═O)R₃, —S(═O)₂R₃, —S(═O)₂NHR₃, —C(═O)CH₃, —OH, —(CH₂)₂OH, 2-amino-propanol, —(CH₂)₃OH, —NH₂, —NH(4-Cl)Ph, —NHNH(Ph), —NHCOCH₃, —N═C(CH₃)₂, —N═C(Ph)₂, or 3-Amino-2-propanol, —(CH₂)₂OC(═O)C₁₃H₂₇, —(CH₂)₂NHC(═O)C₁₃H₂₇, —CH₂(Ph), —(C₅H₁₀), —(CH₂)₂CH₃, —CH₃, —CH₂(2-Cl)Ph-, —(CH₂)₂CH(CH₃)₂, —(CH₂)₂(C₈H₆NCl), —(CH₂)₂(5-chloroindole), —CH₂CH(CH₃)₂—(CH₂)₂NH(C₉H₆N), —CH₂(C₃H₅NBoc), —CH₂(C₃H₅O), —CH₂(C₄H₅)F₂, —C(═O)(3- Cl)Ph-, —(CH₂)₂NH₂, —(CH₂)₂NHBoc, —(CH₂)₃NH₂, —(CH₂)₃NHBoc, —(CH₂)₂(C₈HC₈N), —NH(CH₂)₄CH₃, —CH₂(C₃H₅), —(C₄H₇), —CH₂(C₄H₇), —(C₄H₅)F₂, —C(C₃) 3, —(C₃H₅), —CH₂(3-Cl)Ph-, —CH₂(4-Cl)Ph-, —CH₂(CH)F₂, —(CH₂) 2Ph-, —(CH₂)₃NH(3-CF₃, 4, Cl) (C₅H₂N), -(4-Cl, 3-F)Ph-, —(C₁₆H₁₀), —(C₆H₃O₂)CF₂, -Fluorescein-thiourea, (2-Cl)Ph-, (4-Cl)Ph-, (2-Me)Ph-, (4-Me)Ph-, (2-OMe)Ph-, (4-OMe)Ph-, (2-OCF₃)Ph-, (4-OCF₃)Ph, (2-F)Ph-, (4-F)Ph- or absent;
R₁ is H, aryl, aryl halide, alkyl, cycloalkane, heteroalkyl, alkyl halide, aromatic, heteroaromatic, amino, amine, heterocycloalkane, cycloalkane, fused aromatic or fused heteroaromatic, all of the foregoing being substituted or unsubstituted, (CN)Ph-, (NO₂)Ph-, (CF₃)Ph-, —C(═O)R₃, —C(═)OOR₃, —S(═O)R₃, —S(═O)₂R₃, —S(═O)₂NHR₃, —C(═O)R₃, —C(═)OOR₃, —S(═O)R₃, —S(═O)₂R₃, —S(═O)₂NHR₃, —C(═O)CH₃, —OH, —(CH₂)₂OH, 2-amino-propanol, —(CH₂)₃OH, —NH₂, —NH(4-Cl)Ph, —NHNH(Ph), —NHCOCH₃, —N═C(CH₃)₂, —N═C(Ph)₂, or 3-Amino-2-propanol, —(CH₂)₂OC(═O)C₁₃H₂₇, —(CH₂)₂NHC(═O)C₁₃H₂₇, —CH₂(Ph), —(C₅H₁₀), —(CH₂)₂CH₃, —CH₃, —CH₂(2-Cl)Ph-, —(CH₂)₂CH(CH₃)₂, —(CH₂)₂(C₈H₆NCl), —(CH₂)₂(5-chloroindole), —CH₂CH(CH₃)₂—(CH₂)₂NH(C₉H₆N), —CH₂(C₃H₅NBoc), —CH₂(C₃H₅O), —CH₂(C₄H₅)F₂, —C(═O)(3-Cl)Ph-, —(CH₂)₂NH₂, —(CH₂)₂NHBoc, —(CH₂)₃NH₂, —(CH₂)₃NHBoc, —(CH₂)₂(C₈HC₈N), —NH(CH₂)₄CH₃, —CH₂(C₃H₅), —(C₄H₇), —CH₂(C₄H₇), —(C₄H₅)F₂, —C(C₃) 3, —(C₃H₅), —CH₂(3-Cl)Ph-, —CH₂(4-Cl)Ph-, —CH₂(CH)F₂, —(CH₂) 2Ph-, —(CH₂)₃NH(3-CF₃, 4, Cl) (C₅H₂N), -(4-Cl, 3-F)Ph-, —(C₁₆H₁₀), —(C₆H₃O₂)CF₂, -Fluorescein-thiourea, (2-Cl)Ph-, (4-Cl)Ph-, (2-Me)Ph-, (4-Me)Ph-, (2-OMe)Ph-, (4-OMe)Ph-, (2-OCF₃)Ph-, (4-OCF₃)Ph, (2-F)Ph-, (4-F)Ph- or absent; and
R₃—H, aryl, aryl halide, alkyl, alkoxy, cycloalkane, heteroalkyl, alkyl halide, aromatic, heteroaromatic, amino, amine, heterocycloalkane, cycloalkane, fused aromatic or fused heteroaromatic, all of the foregoing being substituted or unsubstituted or absent.

2. The re RMA of claim 1, wherein R₁ is (Cl)Ph-, and R₂ is (Cl)Ph-, and X is NH.

3. The re RMA of claim 1, wherein R₁ is (2-Cl)Ph-, and R₂ is (2-Cl)Ph-, and X is NH.

4. The re RMA of claim 1, wherein R₁ is (4-Cl)Ph, and R₂ is (4-Cl)Ph-, and X is NH.

5-22. (canceled)

23. A resistance-modifying agent (RMA) according to the compound of Formula I: 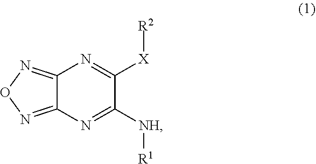
or a stereoisomer, pharmaceutically acceptable salt thereof, wherein,
X is —H, —OH, —S—, —NHCO—, —NH—, —NH₂, —N(OH)—, or —NHOH;
R₂ is aryl, aryl halide, alkyl, cycloalkane, heteroalkyl, heterocycloalkane, cycloalkane, fused aromatic or fused heteroaromatic, alkyl halide, aromatic, heteroaromatic, amino, amine, all of the foregoing being unsubstituted or unsubstituted, —C(═O)R₃, —C(═)OOR₃, —S(═O)R₃, —S(═O)₂R₃, —S(═O)₂NHR₃, Ph, (CN)Ph-, (NO₂)Ph-, (CF₃)Ph-, or absent; and
R₁ aryl, aryl halide, alkyl, cycloalkane, heteroalkyl, heterocycloalkane, cycloalkane, fused aromatic or fused heteroaromatic, alkyl halide, aromatic, heteroaromatic, amino, amine, all of the foregoing being unsubstituted or unsubstituted, —C(═O)R₃, —C(═)OOR₃, —S(═O)R₃, —S(═O)₂R₃, —S(═O)₂NHR₃, Ph, —CN, —NO₂, —CF₃, or absent;
R₃ is H, alkyl, alkoxy, halogen, aromatic, heteroaromatic, cycloalkane, heteroalkyl, heterocycloalkane, cycloalkane, fused aromatic or fused heteroaromatic, all of the foregoing being unsubstituted or unsubstituted or absent.

24-41. (canceled)

42. A pharmaceutical composition comprising a compound of claim 1, and a pharmaceutically acceptable carrier.

43. A method for treating a bacterial infection by a Gram-negative bacteria comprising administering to a subject in need thereof a therapeutically effective amount of the pharmaceutical composition of claim 42, and an antibiotic.

44. The method of claim 43, wherein the bacteria is a Multi-Drug Resistant (MDR) Gram-negative bacteria.

45. The method of claim 44, wherein said MDR Gram-negative bacteria comprises a polymyxin-resistant Gram-negative bacteria selected from the group consisting of a bacteria belonging to: the genus Escherichia, the genus Pseudomonas, the genus Acinetobacter, the genus Salmonella, the genus Klebsiella, the genus Neisseria, the genus Enterobacter, the genus Shigella, the genus Moraxella, the genus Helicobacter, the genus Stenotrophomonas, the genus Bdellovibrio, and the genus Legionella.

46. The method of claim 44, wherein polymyxin-resistant Gram-negative bacteria is selected from the group consisting of: E. coli, S. maltophilia, E. cloacae, K. pneumoniae, A. baumannii, Salmonella spp. and P. aeruginosa.

47. The method of claim 43, wherein said antibiotic comprises a polymyxin antibiotic.

48. The method of claim 48, wherein the polymyxin antibiotic is selected from the group consisting of: colistin, and polymyxin B, or a combination thereof.

49. A pharmaceutical composition comprising a compound of claim 23, and a pharmaceutically acceptable carrier.

50. A method for treating a bacterial infection by a Gram-negative bacteria comprising administering to a subject in need thereof a therapeutically effective amount of the pharmaceutical composition of claim 49, and an antibiotic.

51. The method of claim 50, wherein the bacteria is a Multi-Drug Resistant (MDR) Gram-negative bacteria.

52. The method of claim 51, wherein said MDR Gram-negative bacteria comprises a polymyxin-resistant Gram-negative bacteria selected from the group consisting of a bacteria belonging to: the genus Escherichia, the genus Pseudomonas, the genus Acinetobacter, the genus Salmonella, the genus Klebsiella, the genus Neisseria, the genus Enterobacter, the genus Shigella, the genus Moraxella, the genus Helicobacter, the genus Stenotrophomonas, the genus Bdellovibrio, and the genus Legionella.

53. The method of claim 51, wherein polymyxin-resistant Gram-negative bacteria is selected from the group consisting of: E. coli, S. maltophilia, E. cloacae, K. pneumoniae, A. baumannii, Salmonella spp. and P. aeruginosa.

54. The method of claim 50, wherein said antibiotic comprises a polymyxin antibiotic.

55. The method of claim 54, wherein the polymyxin antibiotic is selected from the group consisting of: colistin, and polymyxin B, or a combination thereof.
